(19) United States
(12) Patent Application Publication
(10) Pub. No.: US 2018/0291457 A1
(43) Pub. Date: Oct. 11, 2018

(54) METAGENOMIC COMPOSITIONS AND METHODS FOR THE DETECTION OF BREAST CANCER

(71) Applicant: THE TRUSTEES OF THE UNIVERSITY OF PENNSYLVANIA, Philadelphia, PA (US)

(72) Inventors: Erle S. ROBERTSON, Wynnewood, PA (US); James C. ALWINE, Media, PA (US)

(21) Appl. No.: 15/565,049

(22) PCT Filed: Apr. 20, 2016
(86) PCT No.: PCT/US2016/028394

§ 371 Date: Oct. 6, 2017

Related U.S. Application Data
(60) Provisional application No. 62/150,126, filed on Apr. 20, 2015.

(51) Int. Cl. C12Q 1/6886

(52) U.S. Cl. CPC C12Q 1/6886

(57) ABSTRACT

The present invention provides compositions and methods for the detection of triple negative breast cancer. Compositions and methods are provided for detecting a metagenomic signature in a tissue sample from a subject that indicates the subject has triple negative breast cancer.

### CROSS REFERENCE TO RELATED APPLICATIONS

This application claims priority to and the benefit of U.S. Provisional Application No. 62/150,126, filed Apr. 20, 2015, which is incorporated herein by reference in its entirety.

### BACKGROUND OF THE INVENTION

The estimated number of new cancer cases in the United States for 2015 is about 1.6 million, with over 500,000 deaths (American Cancer Society, www.cancer.org). Infection with one or more viruses or microorganisms is the third highest contributor to the development of cancer, accounting for at least 20% of tumors (Sawyers et al. (2013) Clin Cancer Res 19, S4-98; de Martel et al. (2012) Lancet Oncol 13, 607-615). Ten viruses (papillomavirus, hepatitis B or C, Polyoma viruses, BK, JC and MCpyV, Epstein-Barr virus, human herpesvirus 8, and T-cell leukemia virus type 1 and type-2), one bacterium (Helicobacter pylori), and two helminthes (schistosomes and liver flukes) have been found to be major contributors to human cancers as etiological agents (de Martel et al. (2012) Lancet Oncol 13, 607-615). Given the many viruses and other microorganisms that are hosted by humans it is likely that their association with cancer is underestimated due to heretofore unrecognized infections or mechanisms. Potentially, microorganisms may have an even greater role in the origin and/or progression of cancers, as well as pathogenesis related to cancer. Thus, knowing the specific viruses and other microbial agents associated with a cancer type (the cancer microbial signature) may provide insights into cause, treatment and diagnosis. For example, persistent infection by one or more infectious agents, resulting in inflammation or alteration of cellular processes, may be involved in the carcinogenic process (Morales-Sanchez & Fuentes-Panana (2014) Viruses 6, 4047-4079). Alternatively, the tumor microenvironment may provide a specialized niche in which these organisms can persist in a way that is difficult to thrive in normal tissue. In either case the identification of unique microbial signatures associated with specific cancers is essential for our understanding of the interplay between the microbiome and cancer, and for diagnosis. Furthermore, it is important to identify pathogens that are associated and can contribute to specific cancers. However, it has been difficult to detect pathogens that are present in low copy number in the tissue sample.

The need to identify pathogenic organisms, including viruses, bacteria, viruses, viroids, bacteria, fungi, helminths, and protozoa, has grown more acute in recent years. To rapidly screen many tumor samples for associated viruses and microorganisms, a microarray-based technology (PathoChip) has been developed that contains probe sets for parallel DNA and RNA detection of viruses and other human pathogenic microorganisms (Baldwin et al. (2014) M Bio 5, e01714-01714). The current version of the PathoChip contains 60,000 probes representing all known viruses, 250 helminths, 130 protozoa, 360 fungi and 320 bacteria. The array contains two types of probes: unique probes for each specific virus and microorganism, and conserved probes which target genomic regions that are conserved between members of a viral family, thereby providing a means for detection of previously uncharacterized members of the family. The PathoChip screening technology includes an amplification step that allows detection of microorganisms and viruses present in low genomic copy number in samples. Thus the PathoChip technology has increased sensitivity relative to other microbiome screening assays, and wider coverage across kingdoms. This allows multiple samples to be rapidly and sensitively screened for the presence of microbial agents.

As de novo cataloging expands the count of species in the human microbiome and characterizes their distributions, metagenomic tools are needed to efficiently identify an agent strongly associated with a disease. The ability to assess a microbiome will be necessary to understand interactions between pathogens, and pathogen interactions with commensal organisms, host genetics, and environmental factors. Considering the thousands of species that comprise the normal human microbiome (Relman. Nature 2012; 486(7402):194-195), it is likely that microorganism communities substantially influence normal physiology as well as the causes of and responses to diseases (Laass et al. Autoimmun Rev 2014), including cancer. These effects are the subject of intense investigation in tissues known to have resident microbiomes such as the gastrointestinal tract (Laass et al. Autoimmun Rev 2014; Major and Spiller. Curr Opin Endocrinol Diabetes Obes 2014; 21(1):15-21; Schwarzberg et al. PLoS One 2014; 9(1):e86708; Scharschmidt and Fischbach. Drug Discov Today Dis Mech 2013; 10(3-4)), skin (Scharschmidt and Fischbach. Drug Discov Today Dis Mech 2013; 10(3-4)) and airway (Martinez et al. Ann Am Thorac Soc 2013; 10 Suppl:S170-179; Segal et al. Ann Am Thorac Soc 2014; 11(1):108-116; Sze et al. H Ann Am Thorac Soc 2014; 11 Suppl 1:S77) and in immune and inflammatory responses (Gjymishka et al. Immunotherapy 2013; 5(12):1357-1366; Kamada and Nunez. Gastroenterology 2014; Koboziev et al. Free Radic Biol Med 2013; 68C:122-133; Ooi et al. PLoS One 2014; 9(1):e86366). Microbiome profiling is also uncovering less obvious roles for microbes and their presence in unexpected locations; examples relevant to cancer include modulation of tumor microenvironments (Iida et al. Science 2013; 342(6161):967-970) and dysbiosis of bacterial populations in breast cancer tissues (Xuan et al, PLoS One 2014; 9(1):e83744).

Accordingly, new compositions and methods based on pathogen detection have the potential to provide a means for diagnosing cancer, especially cancer associated with infectious agents, and for gaining an understanding of the association between cancer and infectious agents. The current invention fulfills these needs.

### SUMMARY OF THE INVENTION

As described herein, the present invention relates to compositions and methods for detecting triple negative breast cancer in a sample. One aspect of the invention includes a method of detecting triple negative breast cancer in a tumor tissue sample from a subject. The method comprises hybridizing a detectably-labeled nucleic acid from the tumor tissue sample to a PathoChip array to generate a first hybridization pattern, then hybridizing a detectably-labeled nucleic acid from a reference sample to a PathoChip array to generate a second hybridization pattern. The reference sample is from an otherwise identical non-tumor tissue from a subject. Next, the first and second hybridization patterns are compared. When the first hybridization pattern is substantially a microbial hybridization signature and the second hybridization pattern is substantially not a microbial hybridization signature, triple negative breast cancer is detected in the tumor tissue sample.

In another aspect, the invention includes a method of detecting triple negative breast cancer in a tumor tissue sample from a subject. The method comprises hybridizing a detectably-labeled nucleic acid from the tumor tissue sample to a first microarray to generate a first hybridization pattern. The first microarray comprises at least three nucleic acid probes selected from the group consisting of SEQ ID NOS: 1-160. The next step is hybridizing a detectably-labeled nucleic acid from a reference sample to a second microarray to generate a second hybridization pattern. The second microarray comprises at least three nucleic acid probes selected from the group consisting of SEQ ID NOS: 1-160. The reference sample is from an otherwise identical non-tumor tissue from a subject. Then, the first and second hybridization patterns are compared. When the first hybridization pattern is substantially a microbial hybridization signature and the second hybridization pattern is substantially not a microbial hybridization signature, triple negative breast cancer is detected in the tumor tissue sample.

In yet another aspect, the invention includes a composition comprising at least three nucleic acid probes selected from the group consisting of SEQ ID NOS: 1-160. Still another aspect of the invention includes a microarray comprising at least three nucleic acid probes selected from the group consisting of SEQ ID NOS: 1-160.

Another aspect of the invention includes a microarray comprising at least three nucleic acid probes. The probes are selected from the group of microbes consisting of Mouse mammary tumor virus (MMTV), Human T-Lymphotropic virus type I (HTLV-1), Fujinami Sarcoma virus (FSV), Simian virus 40 (SV40), John Cunningham virus (JC), Merkel cell Polyomavirus (MCPV), Human Cytomegalovirus (HCMV), Epstein-Barr virus (EBV), Kaposi's sarcoma-associated herpesvirus (KSHV), Human papillomavirus 16 (HPV16), Human papillomavirus 6b (HPV6b), Hepatitis B virus (HBV), Hepatitis C virus (HCV-1), Bovine papular stomatitis virus (BPSV), Pseudocowpox virus (PCP), Taterapox virus (Tatera), Orf virus (Orf), Arcanobacterium, Brevundimonas sp, Sphingobacteria, Providencia, Prevotella, Brucella, Escherichia coli (E. coli), Actinomyces, Mobiluncus, Propiniobacteria, Geobacillus, Rothia, Peptinophilus, Capnocytophaga, Pleistophora, Piedra, Foncecaea, Phialophora, Paecilomyces, Trichuris sp., Toxocara sp., Leishmania sp., Theileria equi (B.equi), Thelazia sp., or Paragonimus sp.

In another aspect, the invention includes a kit comprising at least two three nucleic acid probes. The probes are selected from the group consisting of SEQ ID NOS: 1-160. The kit includes instructional material for use thereof.

In yet another aspect, the invention includes a kit comprising a microarray. The microarray comprises at least three nucleic acid probes. The probes are selected from the group of microbes consisting of MMTV, HTLV-1, FSV, SV40, JC, MCPV, HCMV, EBV, KSHV, HPV16, HPV6b, HBV, HCV-1, BPSV, PCP Tatera, Orf, Arcanobacterium, Brevundimonas sp, Sphingobacteria, Providencia, Prevotella, Brucella, E. coli, Actinomyces, Mobiluncus, Propiniobacteria, Geobacillus, Rothia, Peptinophilus, Capnocytophaga, Pleistophora, Piedra, Foncecaea, Phialophora, Paecilomyces, Trichuris sp., Toxocara sp., Leishmania sp., B.equi, Thelazia sp., Paragonimus sp.

In various embodiments of the above aspects or any other aspect of the invention delineated herein, the microbial hybridization signature is generated by hybridization of the detectably-labeled nucleic acid from the tumor tissue sample to at least three nucleic acid probes on the PathoChip. The probes are from microbes selected from the group consisting of MMTV, HTLV-1, FSV, SV40, JC, MCPV, HCMV, EBV, KSHV, HPV16, HPV6b, HBV, HCV-1, BPSV, PCP Tatera, Orf, Arcanobacterium, Brevundimonas sp, Sphingobacteria, Providencia, Prevotella, Brucella, E. coli, Actinomyces, Mobiluncus, Propiniobacteria, Geobacillus, Rothia, Peptinophilus, Capnocytophaga, Pleistophora, Piedra, Foncecaea, Phialophora, Paecilomyces, Trichuris sp., Toxocara sp., Leishmania sp., B.equi, Thelazia sp., Paragonimus sp.

In another embodiment, the first hybridization pattern is generated by hybridization of the detectably-labeled nucleic acid from the tumor tissue sample to at least three nucleic acid probes on the PathoChip. The probes are selected from the group consisting of SEQ ID NOS: 1-160.

In yet another embodiment, the tumor tissue sample is selected from the group consisting of a biopsy, formalin-fixed, paraffin-embedded (FFPE) sample, or non-solid tumor. In still another embodiment, the subject is human. In certain embodiments, when triple negative breast cancer is detected in the tumor tissue sample from a subject, then the subject is provided with a treatment for triple negative breast cancer. Treatment for triple negative breast cancer can comprise surgery, chemotherapy, or radiotherapy.

In another embodiment the detectably-labeled nucleic acid is labeled with a fluorophore, radioactive phosphate, biotin, or enzyme. In certain embodiments, the fluorophore is Cy3 or Cy5.

In yet another embodiment, the nucleic acid probes in the microarray are selected from a group of about 10 to about 30 microbes and comprise about 3 to about 5 probes per microbe. In another embodiment, the nucleic acid probes in the kit are selected from a group of about 10 to about 30 microbes and comprise about 3 to about 5 probes per microbe.

In certain embodiments the microarray is a biochip, glass slide, bead, or paper.

### BRIEF DESCRIPTION OF THE DRAWINGS

FIGS. 1A-1J depict MiSeq reads aligned to the metagenome of the PathoChip revealing the identity of the targets captured by the selected probes (probe pool VCP, probe pool VSP, probe pool Pox, probe pool B1 and B2, probe pool P1 and P2) during capture sequencing. The genomic location along with the Miseq reads for individual captures are shown. The genomic location of individual accessions, along with the number of MiSeq reads for individual captures are mentioned. The alignment track of IGV displayed the upper coverage track and the lower alignment track. IGV display the paired-end alignments that deviate from expectations by standard color (horizontal black lines). The mismatched bases are also displayed in black on the grey aligned sequence bar that represents the read. The viral signatures and the other microbial signatures captured by the selected probes during capture sequencing are shown.

FIGS. 2A-2D are tables listing the types of probes used for target capture. Nucleotide sequences of the probes are listed in Table 2.

FIGS. 3A-3G, depict the percent probes of candidate organisms showing undetectable, low (>30 to 300), moderate (300-3000) and high (>3000) hybridization signal (Cy3-Cy5) in 100 breast cancer samples (40 individual and 12 pooled) by PathoChip screening. Matched controls (MC) and non-matched controls (NC) are included to show the significant detection of probes in the breast cancer samples vs the controls. FIGS. 3A-3C show the percent detection of specific probes of viral candidates detected in breast cancer samples. FIGS. 3D-3E show the percent detection of bacterial probes detected with low, medium and high hybridization signal in the breast cancer samples. FIG. 3F is a chart showing the percentage of fungal probes detected with low, medium and high hybridization signal in the breast cancer samples. FIG. 3G is a chart showing the percentage of parasitic probes detected with low, medium and high hybridization signal in the breast cancer samples.

FIGS. 4A-4D, depict the detection of viral and microbial signatures associated with triple negative breast cancer samples. FIG. 4A is a heat map of probes (x-axis) hybridized to the tumor samples and both matched (MC) and non-matched control (NC) samples (y-axis) showing hybridization signals (test minus reference) for conserved and specific viral probes detected in the 100 triple negative breast tumor samples. FIG. 4B is a series of graphs showing the percent detection of specific viral signatures in 100 triple negative breast tumor samples ranked according to prevalence and decreasing hybridization signal of the probes to the tumors. FIG. 4C is a heat map of probes (x-axis) hybridized to the tumor samples (y-axis) showing hybridization signals (test minus reference) for conserved and specific bacterial, fungal and parasitic probes detected in the 100 triple negative breast tumor samples. FIG. 4D is series of graphs showing the percent detection of specific microbial signatures in 100 triple negative breast tumor samples ranked according to prevalence and decreasing hybridization signal.

FIG. 5 is a heatmap showing hierarchial clustering of chosen candidate infectious agents in 100 triple negative breast cancer samples. Samples were grouped based on similar viral, bacterial, fungal, and parasitic candidate signature detection.

FIGS. 6A-6C are a series of images showing validation of PathoChip hybridization results by PCR. Primers for PCR amplification were designed from the conserved and specific probes that hybridized to the targets used in the PathoChip screen. The heat map across the cancer and control samples for the probes from which the PCR primers were designed are shown in the left panel for each PCR amplification gel image. Amplified PCR product validated the PathoChip hybridization results. MC: matched control (adjacent non-cancerous breast tissue from breast cancer patients); NC: non-matched control (Breast tissue from healthy individuals). NTC: non-template control-sterile water used to rule out any contamination in the PCR reaction.

FIGS. 7A-7D, depict the capture pool used for nucleic acid capture and MiSeq data analysis. FIG. 7A is a heat map indicating test minus reference signals from the probes (Y-axis) chosen from 4 different analyses. Seven (7) separate captures of target nucleic acids were done using 5 probe pools as indicated. FIGS. 7B-7D are a series of panels showing the individual reads obtained from the MiSeq for the triple negative breast cancer samples. Whole genome amplified DNA plus cDNA was hybridized to a set of biotinylated conserved and specific viral, bacterial, fungal, parasitic and viroid probes, captured on streptavidin beads, and used for tagmentation library preparation and deep sequencing with paired-end 250-nt reads. The MiSeq was done on libraries generated by capture sequences using viral conserved probes (capture probe pool VCP), viral specific probes (capture probe pool VSP), pox virus probes (capture probe pool Pox), bacterial probes (capture probe pool B1 and B2), fungal/parasitic and viroid probes (capture probe pool P1 and P2). The Miseq reads from individual capture when aligned with the metagenome of PathoChip (Chip probes) was found to cluster mostly at the capture probe regions of the represented organisms. The genomic location along with the number of MiSeq reads are shown on the figure and represents the genomic co-ordinates.

FIGS. 8A-8F are a listing of MiSeq reads of candidates in 7 different capture reactions namely bacterial (B1 and B2), parasitic-fungal-viroid (P1 and P2), pox conserved (pox), viral specific (VSP) and viral conserved (VCP) probe. The reads that map to each organism are summarized across the 7 capture sequencing (B1, B2, P1, P2, Pox, VCP and VSP, respectively). Specifically the total numbers of reads were counted that aligned to the whole species (*_org), to the capture probe regions (*_probe), and to the out-of-probe regions (*_outprobe). See for example, organism DQ118536.1, detected by P1 capture sequencing. There are 168 reads (P1_org) aligned to this organism, of which 160 reads (p1_probe) aligned to the capture probe region and the remaining 8 reads (P1_outprobe) aligned to out-of-capture-probe regions. For each organism, the score column gives the number of capture sequencing under which reads are mapped to both the capture probe regions and the out-of-probe regions. For example, the score of organism DQ118536.1 is 2 because reads were found to map to both the probed regions and out-of-probe regions by P1 and P2 capture sequencing. The total number of reads mapping to the capture probe regions in all the 7 capture sequencing conditions were summed in the Probe_score column. Those candidate organisms with reads that mapped to the capture probe regions (Probe_score>0) are listed and ranked by the score column.

### DETAILED DESCRIPTION OF THE INVENTION

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the articles “a”, “an” and “the” include plural referents unless context clearly indicates otherwise. By way of example, “an element” means one element or more than one element.

As used herein, the term “about” will be understood by persons of ordinary skill in the art and will vary to some extent on the context in which it is used. As used herein when referring to a measurable value such as an amount, a concentration, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

A “biomarker” or “marker” as used herein generally refers to a nucleic acid molecule, clinical indicator, protein, or other analyte that is associated with a disease. In certain embodiments, a nucleic acid biomarker is indicative of the presence in a sample of a pathogenic organism, including but not limited to, viruses, viroids, bacteria, fungi, helminths, and protozoa. In various embodiments, a marker is differentially present in a biological sample obtained from a subject having or at risk of developing a disease (e.g., an infectious disease) relative to a reference. A marker is differentially present if the mean or median level of the biomarker present in the sample is statistically different from the level present in a reference. A reference level may be, for example, the level present in an environmental sample obtained from a clean or uncontaminated source. A reference level may be, for example, the level present in a sample obtained from a healthy control subject or the level obtained from the subject at an earlier timepoint, i.e., prior to treatment. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. The differential presence of a marker of the invention in a subject sample can be useful in characterizing the subject as having or at risk of developing a disease (e.g., an infectious disease), for determining the prognosis of the subject, for evaluating therapeutic efficacy, or for selecting a treatment regimen.

By “agent” is meant any nucleic acid molecule, small molecule chemical compound, antibody, or polypeptide, or fragments thereof.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

By “biologic sample” is meant any tissue, cell, fluid, or other material derived from an organism.

By “capture reagent” is meant a reagent that specifically binds a nucleic acid molecule or polypeptide to select or isolate the nucleic acid molecule or polypeptide.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “detectable moiety” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “fragment” is meant a portion of a nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 10, 15, 20, 30, 40, 50, 60, 70, 80, 90, or 100 nucleotides.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “reference” is meant a standard of comparison. As is apparent to one skilled in the art, an appropriate reference is where an element is changed in order to determine the effect of the element. In one embodiment, the level of a target nucleic acid molecule present in a sample may be compared to the level of the target nucleic acid molecule present in a clean or uncontaminated sample. For example, the level of a target nucleic acid molecule present in a sample may be compared to the level of the target nucleic acid molecule present in a corresponding healthy cell or tissue or in a diseased cell or tissue (e.g., a cell or tissue derived from a subject having a disease, disorder, or condition).

By “marker profile” is meant a characterization of the signal, level, expression or expression level of two or more markers (e.g., polynucleotides).

By the term “microbe” is meant any and all organisms classed within the commonly used term “microbiology,” including but not limited to, bacteria, viruses, fungi and parasites.

By the term “microarray” is meant a collection of nucleic acid probes immobilized on a substrate. As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring. Nucleic acid molecules useful in the methods of the invention include any nucleic acid molecule that specifically binds a target nucleic acid (e.g., a nucleic acid biomarker). Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95%, 96%, 97%, 98%, or even 99% or more identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

As used herein, the term “sample” includes a biologic sample such as any tissue, cell, fluid, or other material derived from an organism.

By “specifically binds” is meant a compound (e.g., nucleic acid probe or primer) that recognizes and binds a molecule (e.g., a nucleic acid biomarker), but which does not substantially recognize and bind other molecules in a sample, for example, a biological sample.

By the term “substantially microbial hybridization signature” is a relative term and means a hybridization signature that indicates the presence of more microbes in a tumor sample than in a reference sample.

By the term “substantially not a microbial hybridization signature” is a relative term and means a hybridization signature that indicates the presence of less microbes in a reference sample than in a tumor sample.

By “subject” is meant a mammal, including, but not limited to, a human or non-human mammal, such as a bovine, equine, canine, ovine, feline, mouse, or monkey. The term “subject” may refer to an animal, which is the object of treatment, observation, or experiment (e.g., a patient).

By “target nucleic acid molecule” is meant a polynucleotide to be analyzed. Such polynucleotide may be a sense or antisense strand of the target sequence. The term “target nucleic acid molecule” also refers to amplicons of the original target sequence. In various embodiments, the target nucleic acid molecule is one or more nucleic acid biomarkers.

By the term “tumor tissue sample” is meant any sample from a tumor in a subject including any solid and non-solid tumor in the subject.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compounds, compositions, or methods provided herein can be combined with one or more of any of the other compositions and methods provided herein. It is also to be understood that the terminology used herein is for the purpose of describing particular embodiments only, and is not intended to be limiting.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Other features and advantages of the invention will be apparent from the following description of the desirable embodiments thereof, and from the claims.

### Description

The present invention features compositions and methods for the detection or diagnosis of triple negative breast cancer in a subject comprising detecting the presence of genetic material from one or more infection agents in a tissue sample from the subject. Metagenomics signatures comprising detecting genetic material from a number of viral, bacterial, fungal, and parasitic infectious agents were identified that indicate that a subject has triple negative breast cancer.

As described herin, the PathoChip approach was used to screen 100 triple negative breast cancer (TNBC) samples as well as 20 matched and 20 unmatched controls. To rapidly screen many tumor samples for associated viruses and microorganisms we developed a microarray-based approach (PathoChip) containing probe sets for parallel DNA and RNA detection of viruses and other human pathogenic microorganisms (Baldwin et al. (2014) M Bio 5, e01714-01714). The current version of the PathoChip contains 60,000 probes representing all known viruses, 250 helminths, 130 protozoa, 360 fungi and 320 bacteria. The array contains two types of probes: unique probes for each specific virus and microorganism, and conserved probes which target genomic regions that are conserved between members of a viral family, thereby providing a means for detection of previously uncharacterized members of the family. The PathoChip screening technology includes an amplification step that allows detection of microorganisms and viruses present in low genomic copy number in samples. Thus the PathoChip technology has increased sensitivity relative to other microbiome screening assays, and wider coverage across kingdoms. This allows multiple tumor samples to be rapidly and sensitively screened for the presence of microbial agents.

Probes were identified that represent virus and other microorganism sequences significantly detected in the breast cancer samples compared to the controls. These probes were used for both PCR verification, and as capture reagents on magnetic beads to select hybridizing sequences from the breast cancer samples, which were sequenced by miSeq for additional verification. The data establish unique microbial signatures for triple negative breast cancer.

### Breast Cancer and Triple Negative Breast Cancer (TNBC)

Breast cancer is one of the most prevalent cancers: in 2015 an estimated 200,000 new cases will be diagnosed in the US resulting in over 40,000 deaths (see e.g., http://seer.cancer.gov/statfacts/html/breast.html). Breast cancers are categorized on the basis of presence or absence of certain hormone and growth receptors. There are 4 major types: Endocrine receptor (estrogen or progesterone receptor) positive, human epidermal growth factor receptor 2 (Her2) positive, triple positive (estrogen, progesterone and HER2 receptor positive) and triple negative (absence of estrogen, progesterone and HER2 receptors) (www.webmd.com/breast-cancer). The later form of breast cancer cannot be treated by endocrine therapy and is the most aggressive form of the disease (http://www.cancercenter.com). Studies have been devoted to genes mutated in those genetically pre-disposed to breast cancer (e.g. BRCA1/2 and others) (Shiovitz and Korde (2015) Ann Oncol 20; Cornejo-Moreno et al. (2014) Isr Med Assoc J 16, 787-792; Sun et al. (2015) Int J Mol Sci 16, 4121-4135; Chacon-Cortes et al., (2015) Tumour Biol 14, 14), as well as other factors like family history (Pilato et al. (2014) J Hum Genet 59, 51-53), ethnicity (Tehranifar et al. (2015) Am J Epidemiol 181, 204-212), obesity (Kruk (2014) Asian Pac J Cancer Prev 15, 9579-9586), breast tissue density (Yaghjyan et al. (2015) Breast Cancer Res Treat 13, 13), gender (Sherman and Lane (2014) J Cancer Educ 17, 17) environmental factors (Hiatt R A, Haslam S Z, & Osuch J (2009) Environ Health Perspect 117, 1814-1822) and factors related to lifestyle (Kruk (2014) Asian Pac J Cancer Prev 15, 9579-9586) that play a major role in the development and progression of these cancers. However, less emphasis has been devoted to determining the association of viruses and microorganisms with breast cancer, although several studies have shown an association with herpesviruses, polyomaviruses, papillomaviruses and retroviruses (Shiovitz and Korde (2015) Ann Oncol 20).

### Metagenomic Signatures and Triple Negative Breast Cancer

In the present application, predominant viral, bacterial, fungal and parasitic genomic sequences were detected in 100 triple negative breast cancer samples using the PathoChip array which contains a set of 60,000 probes that cover all known viral agents as well as human pathogenic bacterial, fungi and parasites. This sensitive approach detected multiple viruses and micro-organisms in individual breast cancer samples. These results were validated by PCR and target capture sequencing. Hierarchical analysis shows that at least two major microbial signatures can be found within the TNBC samples tested. Importantly, the data provide limited information about how these viruses and other microbial agents are associated with the tumor tissue or tumor micro-environment. The data do not suggest that these viruses and microorganisms are causative or contribute to the development of TNBC. While these viruses and microorganisms could contribute to cancer pathology, it is also possible that the tumor tissue and the tumor microenvironment provide an amiable niche for them to persist. At the very least, the presence of these viral and micro-organismal signatures provide diagnostic capabilities.

Interestingly, the TNBC samples fell into hierarchical groups showing at least two distinct microbial signatures. One hierarchical signature was prevalent in viruses: a herpesvirus-signature (primarily β- and γ-herpesvirus-like); a parapoxvirus signature (parapox virus family-like); flavivirus (hepatitis C- and GB-like); polyoma (JC-MCPV- and SV40-like); retrovirus (MMTV-, HERV-K-, HTLV-like); hepadnavirus (hepatitis B-like) and papillomavirus (HPV-2, 6b and 18-like). This hierarchical signature also tended to be higher in parasite signatures representative of the Trichuris, Toxocara, Leishmania, Babesia and Thelazia families. There has been one report on the association of parasites with metastatic breast cancer (Schafer A (1969) Experientia 25, 729-732). A second prominent hierarchical signature showed fewer viruses and parasites but a higher bacterial content indicated by representatives of a number of families (Actinomycetaceae, Caulobacteriaceae, Sphingobacteriaceae, Enterobacteriaceae, Prevotellaceae, Brucellaceae, Bacillaceae, Peptostreptococcaceae, Flavobacteriaceae), some of which have been associated with cancers (Han and Andrade (2005) J Antimicrob Chemother 55, 853-859; Dobinsky et al. (1999) Eur J Chn Microbiol Infect Dis 18, 804-806; Alison et al. (2014) EJSO 40, 650-651; Gupta et al. (2012) Breast Care (Basel) 7, 153-154). Fungal signatures could be found relatively equally between the two hierarchical signatures and suggested representatives of the Pleistophora, Piedraia, Fonsecaea, and Phialophora families.

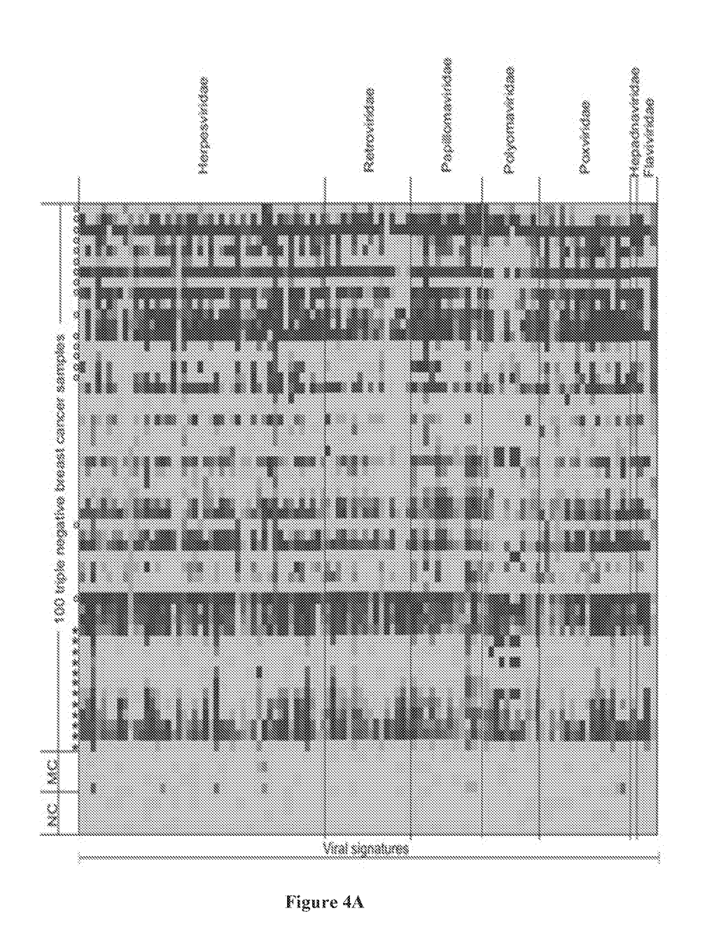

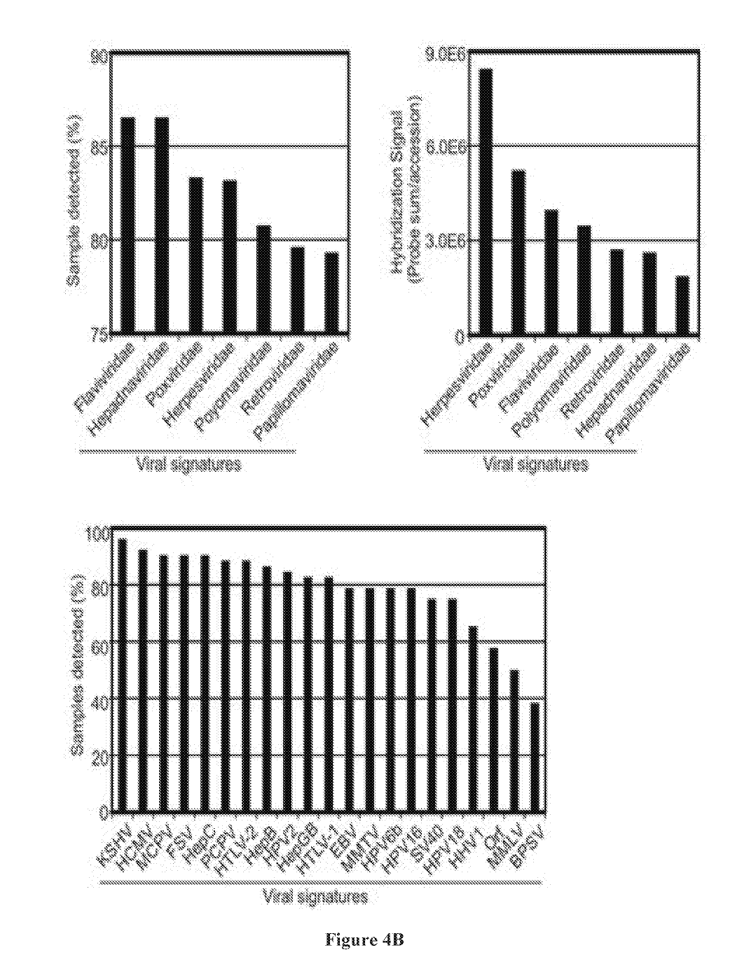

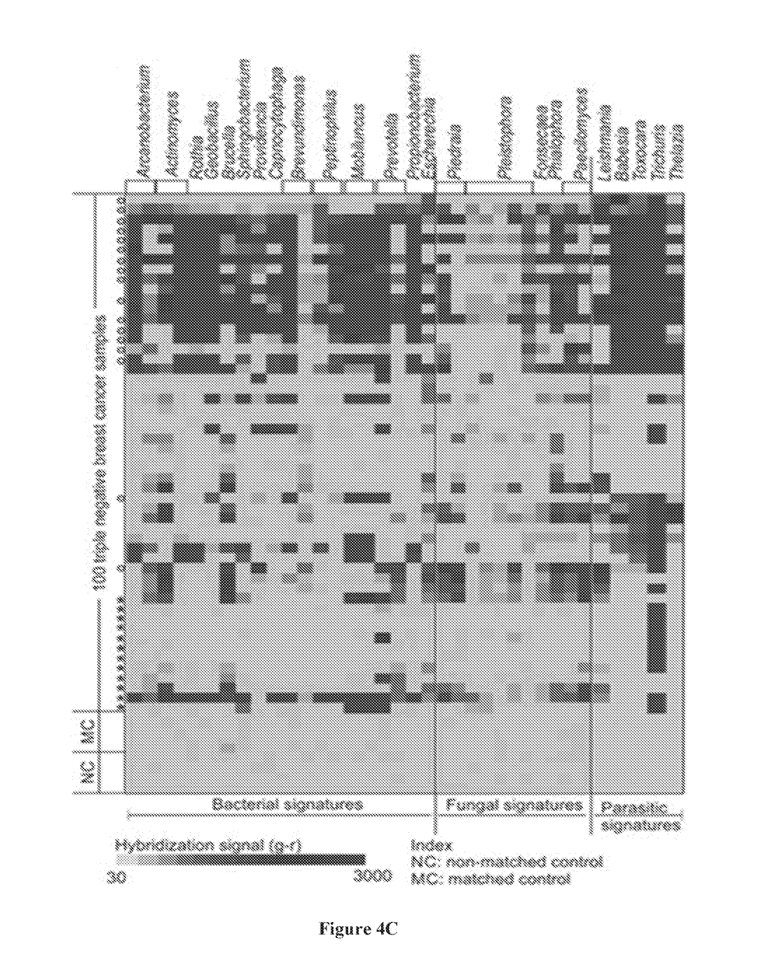

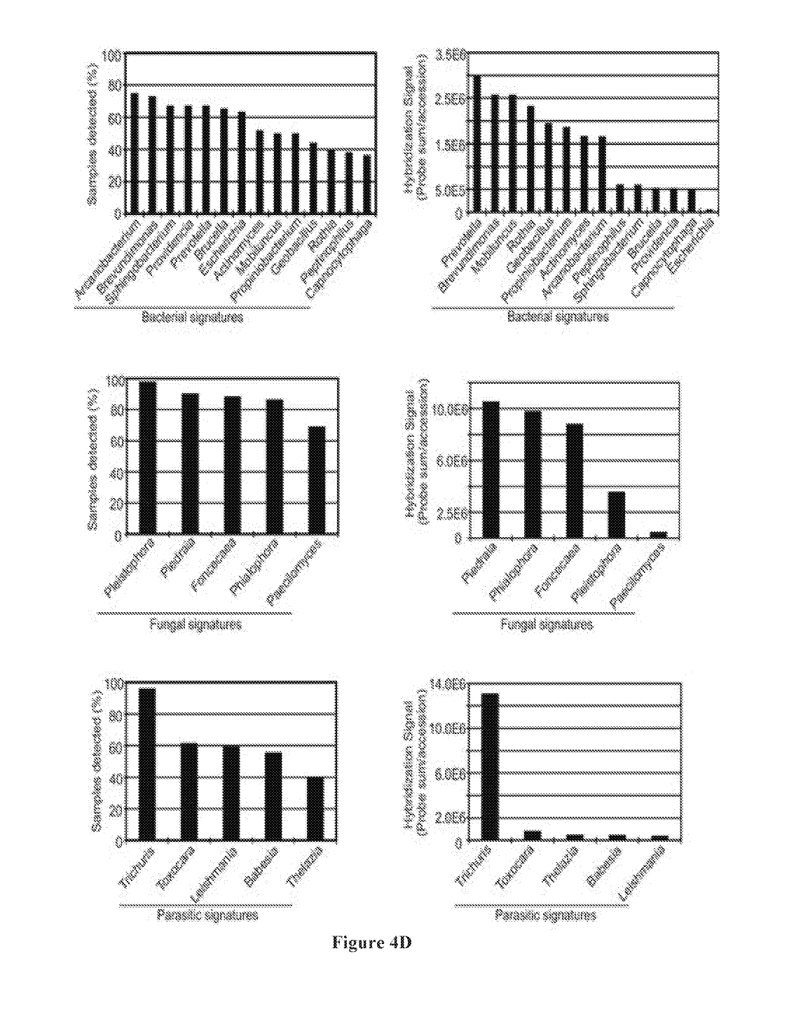

The PathoChip screen also provided some surprising results. For example, detection of the sequences related to Okra mosaic virus (Stephan et al. (2008) Virus Genes 36, 231-240) and citrus viroid V (FIGS. 4A-4D and Table 5). Interestingly, the detection of RNA for viroids is supported by a study which suggested intra-nuclear viroids in breast cancer (Schafer (1969) Experientia 25, 729-732). Additionally, dietary raw fruits and vegetables expose individivals to large numbers of plant viruses and viriods, and some may persist. The screen also detected genomic sequences similar to a baculovirus. Without being bound to a particular theory, it is quite possible that variants of insect and plant virus can persist in human under specific situations.

Thus as more studies can be done in fresh tissue the TNBC microbial signature may be broadened. Because RNA viral genomes are more prone to degradation in FFPE samples, the screen may be biased toward DNA viruses since. Nevertheless the data clearly indicate that a microbial signature can be delineated in TBNC and this signature is underrepresented in normal tissue.

In one embodiment, the invention includes a method of detecting triple negative breast cancer in a tumor tissue sample from a subject. The method comprises the steps of hybridizing a detectably-labeled nucleic acid from the tumor tissue sample to a PathoChip array to generate a first hybridization pattern, and hybridizing a detectably-labeled nucleic acid from a reference sample to a PathoChip array to generate a second hybridization pattern. The reference sample is from an otherwise identical non-tumor tissue from a subject. Next, the first and second hybridization patterns are compared. When the first hybridization pattern is substantially a microbial hybridization signature and the second hybridization pattern is substantially not a microbial hybridization signature, triple negative breast cancer is detected in the tumor tissue sample.

In another embodiment of the method, the microbial hybridization signature is generated by hybridization of the detectably-labeled nucleic acid from the tumor tissue sample to at least three nucleic acid probes on the PathoChip. The number of nucleic acid probes useful in the methods of the invention may be at least 3 probes, at least 10 probes, at least 30 probes, at least 90 probes, at least 120 probes, at least 140 probes, at least 160 probes, or any and all numbers of probes therebetween. Use of these numbers of nucleic acid probes apply to each and every method, composition, and kit described herein.

In one embodiment of the method, the probes are from microbes selected from the group consisting of: MMTV, HTLV-1, FSV, SV40, JC, MCPV, HCMV, EBV, KSHV, HPV16, HPV6b, HBV, HCV-1, BPSV, PCP Tatera, Orf, Arcanobacterium, Brevundimonas sp, Sphingobacteria, Providencia, Prevotella, Brucella, E. coli, Actinomyces, Mobiluncus, Propiniobacteria, Geobacillus, Rothia, Peptinophilus, Capnocytophaga, Pleistophora, Piedra, Foncecaea, Phialophora, Paecilomyces, Trichuris sp., Toxocara sp., Leishmania sp., B.equi, Thelazia sp., Paragonimus sp.

The method can also include steps wherein the first hybridization pattern is generated by hybridization of the detectably-labeled nucleic acid from the tumor tissue sample to at least three nucleic acid probes on the PathoChip. In this case, the probes are selected from the group consisting of SEQ ID NOS: 1-160.

In another embodiment, the invention includes a method of detecting triple negative breast cancer in a tumor tissue sample from a subject, comprising the steps of hybridizing a detectably-labeled nucleic acid from the tumor tissue sample to a first microarray to generate a first hybridization pattern and hybridizing a detectably-labeled nucleic acid from a reference sample to a second microarray to generate a second hybridization pattern, The microarrays are comprised of at least three nucleic probes selected from the group consisting of SEQ ID NOS: 1-160. The reference sample is from an otherwise identical non-tumor tissue from a subject. Next, the first and second hybridization patterns are compared. If the first hybridization pattern is substantially a microbial hybridization signature and the second hybridization pattern is substantially not a microbial hybridization signature, then triple negative breast cancer is detected in the tumor tissue sample.

The tumor tissue sample can be from a biopsy, paraffin-embedded (FFPE) sample, or non-solid tumor. And, the subject can be a human. The detectably-labeled nucleic acid can be labeled with a fluorophore (such as Cy3 or Cy5), a radioactive phosphate, biotin, or an enzyme.

The methods can also include providing the subject with a treatment for triple negative breast cancer when triple negative breast cancer is detected in the tumor tissue sample from the subject. Examples of treatments include, but are not limited to, surgery, chemotherapy, or radiotherapy.

### Target Nucleic Acid Molecules

Methods and compositions of the invention are useful for the identification of a target nucleic acid molecule in a biological to be analyzed. Target sequences are amplified from any biological sample that comprises a target nucleic acid molecule. Such samples may comprise fungi, spores, viruses, or cells (e.g., prokaryotes, eukaryotes, including human). Such samples may comprise viral, bacterial, fungal, and parasitic nucleic acid molecules. In specific embodiments, compositions and methods of the invention detect one or more nucleic acid sequences from one or more pathogenic organisms, including viruses, viroids, bacteria, fungi, helminths, and/or protozoa.

In one embodiment, a sample is a biological sample, such as a tissue or tumor sample. The level of one or more polynucleotide biomarkers (e.g., to detect or identify viruses, viroids, bacteria, fungi, helminths, and/or protozoa) is measured in the biological sample. In one embodiment, the biological sample is a tissue sample that includes a breast cell or tumor cell, for example, from a biopsy or formalin-fixed, paraffin-embedded (FFPE) sample. Exemplary test samples also include body fluids (e.g. blood, serum, plasma, amniotic fluid, sputum, urine, cerebrospinal fluid, lymph, tear fluid, feces, or gastric fluid), feces, tissue extracts, and culture media (e.g., a liquid in which a cell, such as a pathogen cell, has been grown). If desired, the sample is purified prior to detection using any standard method typically used for isolating a nucleic acid molecule from a biological sample. In one embodiment, a target nucleic acid of a pathogen is amplified by primer oligonucleotides to detect the presence of the nucleic acid sequence of an infectious agent in the sample. Such nucleic acid sequences may derive from pathogens including fungi, bacteria, viruses and yeast.

Target nucleic acid molecules include double-stranded and single-stranded nucleic acid molecules (e.g., DNA, RNA, and other nucleobase polymers known in the art capable of hybridizing with a nucleic acid molecule described herein). RNA molecules suitable for detection with a detectable oligonucleotide probe or detectable primer/template oligonucleotide of the invention include, but are not limited to, double-stranded and single-stranded RNA molecules that comprise a target sequence (e.g., messenger RNA, viral RNA, ribosomal RNA, transfer RNA, microRNA and microRNA precursors, and siRNAs or other RNAs described herein or known in the art). DNA molecules suitable for detection with a detectable oligonucleotide probe or primer/template oligonucleotide of the invention include, but are not limited to, double stranded DNA (e.g., genomic DNA, plasmid DNA, mitochondrial DNA, viral DNA, and synthetic double stranded DNA). Single-stranded DNA target nucleic acid molecules include, for example, viral DNA, cDNA, and synthetic single-stranded DNA, or other types of DNA known in the art. In general, a target sequence for detection is between about 30 and about 300 nucleotides in length (e.g., 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300 nucleotides). In a specific embodiment the target sequence is about 60 nucleotides in length. A target sequence for detection may also have at least about 70, 80, 90, 95, 96, 97, 98, 99, or even 100% identity to a probe sequence. Probe sequences may be longer or shorter than the target sequence. For example, a 60-nucleotide probe may hybridize to at least about 44 nucleotides of a target sequence.

In particular embodiments, a biomarker is a biomolecule (e.g., nucleic acid molecule) that is differentially present in a biological sample. For example, a biomarker is taken from a subject of one phenotypic status (e.g., having triple negative breast cancer) as compared with another phenotypic status (e.g., not having triple negative breast cancer). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for characterizing a disease (e.g., having triple negative breast cancer).

### Probe Selection

Sets of probes selected for detecting multiple target nucleic acid molecules (e.g., corresponding to multiple bioorganisms) are used in the methods of the invention. In various embodiments, the set of probes is based on the construction of a metagenome and its use to select probes that identify target nucleic acid molecules associated with an infectious agent. As used herein “metagenome” refers to genetic material from more than one organism, e.g., in an environmental sample. The metagenome is used to select the sets of probes and/or to validate probe sets. In some embodiments, the metagenome comprises the sequences or genomes of about 20, 30, 40, 50, 60, 70, 80, 90, 100, 200, 300, 400, 500, 1000, 1500, 2000 or more organisms. In one example, the nucleic acid sequences of thousands of organisms were linked to generate a metagenome comprising 58 chromosomes.

### Discrete Metagenome Probe Selection

A. Download individual genomes, genes and partial sequences into a local database of accessions
B. Mask low complexity sequences using bioinformatic tools. In one example, low complexity sequences are masked using mdust (http://doc.bioperl.org/bioperl-run/lib/Bio/Tools/Run/Mdust.html) followed by BLASTN 2.0MP-WashU31 identification of unique regions in viral accessions.
C. BLASTN sequence comparison of each accession against all other accessions
D. Identify specific target regions within each accession
   1. 250-300 bp regions
   2. No more than 50 contiguous nucleotides with 70% or greater sequence homology to any other accession or to the human genome
E. Supplement specific targets
   1. Identify any accessions with zero or one target region
   2. Relax stringency parameters to no more than 30 contiguous nucleotides with 50% or greater sequence homology to any other accession, but no more than 50 contiguous nucleotides with 70% or greater sequence homology to human genome
   3. Re-run target region identification on accession subset from 1.E.1.
F. Identify conserved target regions
   1. 70-300 bp regions that have 70% or greater homology with at least one other accession
   2. Remove conserved targets with 50 or more contiguous nucleotides with 70% or greater sequence homology to human genome
G. Choose probes
   1. Run Agilent array CGH probe selection algorithm on specific and conserved target regions
   2. Rank probes by Agilent design score
   3. Select 1-3 highest ranking probes from 1-5 specific target regions in each accession
   4. Select 1-3 highest ranking probes from each conserved target region

### Concatenated Metagenome Probe Selection

A. Download individual genomes, genes and partial sequences into a local database of accessions
B. Compile all accessions into a single concatenated metagenome to facilitate use of genomics bioinformatics tools
   1. Place 100 nonspecific nucleotides (“N”) as spacers between each accession
   2. Join accessions and spacers into chromosomes of 6-10 million bases
C. Run Agilent array CGH probe selection algorithm for specificity within the metagenome
D. Filter probes for specificity against human, mouse, and/or other mammalian genomes
E. Choose specific probes
   1. Rank probes by Agilent design score
   2. Select 10-20 highest ranking probes from each accession
   3. Require at least 100 bp separation between probes
F. Choose conserved probes
   1. Identify conserved regions as in l.F.
   2. Select 5-10 highest ranking probes from each conserved region
   3. Require at least100 bp separation between probes
G. Empirical probe selection
   1. Manufacture microarrays containing all specific and conserved probes
   2. Hybridize microarrays to labeled human DNA
   3. Select 5-10 specific probes from each accession with lowest cross-hybridization signal
   4. Select 3-5 conserved probes from each conserved regions with lowest cross-hybridization signal

In one embodiment, the invention includes at least two nucleic acid probes selected from the group consisting of SEQ ID NOS: 1-160.

### Sample Preparation

The invention provides a means for analyzing multiple types of nucleic acids present in a sample, including DNA and RNA. In various embodiments, sample preparation involves extracting a mixture of nucleic acid molecules (e.g., DNA and RNA). In other embodiments, sample preparation involves extracting a mixture of nucleic acids from multiple organisms, cell types, infectious agents, or any combination thereof. In one embodiment, sample preparation involves the workflow below.
A. Fragment genomic DNA
B. Convert total RNA to first strand cDNA by random-primed reverse transcriptase
C. Label genomic DNA with biotin or fluorescent dye by chemical or enzymatic incorporation
D. Label cDNA with biotin or fluorescent dye by chemical or enzymatic incorporation
E. Label a mixture of genomic DNA and cDNA in the same chemical or enzymatic reaction
F. Mix C+D and co-hybridize to microarray of probes
G. Hybridize E to microarray of probes
H. Amplify targeted genomic DNA
   1. Use whole-genome amplification (GE GenomiPhi, Sigma WGA, NuGEN Ovation DNA) to non-specifically amplify genomic DNA
   2. Use amplified products as input for 4.C, or 4.E.
I. Amplify targeted total RNA
   1. Use whole-transcriptome amplification (Sigma WTA, Ambion in vitro transcription, NuGEN Ovation RNA) to non-specifically amplify total RNA
   2. Use amplified products as input.
      The samples are hybridized to the microarray (e.g., PathoChip), and the microarrays are washed at various stringencies. Microarrays are scanned for detection of fluorescence. Background correction and inter-array normalization algorithms are applied. Detection thresholds are applied. The results are analyzed for statistical significance.

### Nucleic Acid Amplification

Target nucleic acid sequences are optionally amplified before being detected. The term “amplified” defines the process of making multiple copies of the nucleic acid from a single or lower copy number of nucleic acid sequence molecule. The amplification of nucleic acid sequences is carried out in vitro by biochemical processes known to those of skill in the art. Prior to or concurrent with identification, the viral sample may be amplified by a variety of mechanisms, some of which may employ PCR. For example, primers for PCR may be designed to amplify regions of the sequence. For RNA viruses a first reverse transcriptase step may be used to generate double stranded DNA from the single stranded RNA. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675. The sample may be amplified on the array. See, for example, U.S. Pat. No. 6,300,070 and U.S. Ser. No. 09/513,300.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed PCR (CP-PCR) (U.S. Pat. No. 4,437,975), arbitrarily primed PCR (AP-PCR) (U.S. Pat. Nos. 5,413,909, 5,861,245) and nucleic acid based sequence amplification (NABSA) (see, U.S. Pat. Nos. 5,409,818, 5,554,517, and 6,063,603). Other amplification methods that may be used are described in, U.S. Pat. Nos. 5,242,794, 5,494,810, 4,988,617 and in U.S. Ser. No. 09/854,317.

Additional methods of sample preparation and techniques for reducing the complexity of a nucleic acid sample are described in Dong et al., Genome Research 11, 1418 (2001), in U.S. Pat. Nos. 6,361,947, 6,391,592 and U.S. Ser. Nos. 09/916,135, 09/920,491 (US Patent Application Publication 20030096235), Ser. No. 09/910,292 (US Patent Application Publication 20030082543), and Ser. No. 10/013,598.

### Detection of Biomarkers

The biomarkers of this invention can be detected by any suitable method. The methods described herein can be used individually or in combination for a more accurate detection of the biomarkers. Methods for conducting polynucleotide hybridization assays have been developed in the art. Hybridization assay procedures and conditions will vary depending on the application and are selected in accordance with the general binding methods known including those referred to in: Sambrook and Russell, Molecular Cloning: A Laboratory Manual (3^{rd} Ed. Cold Spring Harbor, N.Y, 2001); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623. A data analysis algorithm (E-predict) for interpreting the hybridization results from an array is publicly available (see Urisman, 2005, Genome Biol 6:R78).

In one embodiment, the hybridized nucleic acids are detected by detecting one or more labels attached to, or incorporated within, the sample nucleic acids. The labels may be attached or incorporated by any of a number of means well known to those of skill in the art. In one embodiment, the label is simultaneously incorporated during the amplification step in the preparation of the sample nucleic acids. Thus, for example, PCR with labeled primers or labeled nucleotides will provide a labeled amplification product. In another embodiment, transcription amplification, as described above, using a labeled nucleotide (e.g. fluorescein-labeled UTP and/or CTP) incorporates a label into the transcribed nucleic acids. In another embodiment PCR amplification products are fragmented and labeled by terminal deoxytransferase and labeled dNTPs. Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example, nick translation or end-labeling (e.g. with a labeled RNA) by kinasing the nucleic acid and subsequent attachment (ligation) of a nucleic acid linker joining the sample nucleic acid to a label (e.g., a fluorophore). In another embodiment label is added to the end of fragments using terminal deoxytransferase.

Detectable labels suitable for use in the present invention include any composition detectable by spectroscopic, photochemical, biochemical, immunochemical, electrical, optical or chemical means. Useful labels in the present invention include, but are not limited to: biotin for staining with labeled streptavidin conjugate; anti-biotin antibodies, magnetic beads (e.g., Dynabeads™.); fluorescent dyes (e.g., fluorescein, texas red, rhodamine, green fluorescent protein, and the like); radiolabels (e.g., 3H, ¹²⁵I, ³⁵S, ⁴C, or ³²P); phosphorescent labels; enzymes (e.g., horse radish peroxidase, alkaline phosphatase and others commonly used in an ELISA); and colorimetric labels such as colloidal gold or colored glass or plastic (e.g., polystyrene, polypropylene, latex, etc.) beads. Patents teaching the use of such labels include U.S. Pat. Nos. 3,817,837, 3,850,752, 3,939,350, 3,996,345, 4,277,437, 4,275,149 and 4,366,241.

Means of detecting such labels are well known to those of skill in the art. Thus, for example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964).

### Detection by Microarray

In aspects of the invention, a sample is analyzed by means of a microarray (also known as a biochip). The nucleic acid molecules of the invention are useful as hybridizable array elements in a microarray. Microarrays generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. U.S. Pat. Nos. 5,800,992 and 6,040,138 describe methods for making arrays of nucleic acid probes that can be used to detect the presence of a nucleic acid containing a specific nucleotide sequence. Methods of forming high-density arrays of nucleic acids, peptides and other polymer sequences with a minimal number of synthetic steps are known. The nucleic acid array can be synthesized on a solid substrate by a variety of methods, including, but not limited to, light-directed chemical coupling, and mechanically directed coupling. For additional descriptions and methods relating to resequencing arrays see U.S. patent application Ser. Nos. 10/658,879, 60/417,190, 09/381,480, 60/409,396, and U.S. Pat. Nos. 5,861,242, 6,027,880, 5,837,832, 6,723,503.

One embodiment of the invention includes a microarray comprising at least two nucleic acid probes selected from the group consisting of SEQ ID NOS: 1-160. The microarray can be a biochip, or on a glass slide, bead, or paper.

### Detection by Nucleic Acid Biochip

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application W095/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure. Exemplary nucleic acid molecules useful in the invention include polynucleotides that specifically bind nucleic acid biomarkers to one or more pathogenic organisms, and fragments thereof.

A nucleic acid molecule (e.g. RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell or population of cells isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In other embodiments, wash steps will occur at 68 C in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Diagnostic Assays

The present invention provides a number of diagnostic assays that are useful for the identification or characterization of a disease or disorder (e.g., triple negative breast cancer), or a propensity to develop such a condition. In one embodiment, triple negative breast cancer is characterized by quantifying the level of one or more biomarkers from one or more pathogenic organisms, including viruses, viroids, bacteria, fungi, helminths, and protozoa. While the examples provided below describe specific methods of detecting levels of these markers, the skilled artisan appreciates that the invention is not limited to such methods. Marker levels are quantifiable by any standard method, such methods include, but are not limited to real-time PCR, Southern blot, PCR, and/or mass spectroscopy.

The level of any two or more of the markers described herein defines the marker profile of a disease, disorder, condition. The level of marker is compared to a reference. In one embodiment, the reference is the level of marker present in a control sample obtained from a patient that does not have triple negative breast cancer. In another embodiment, the reference is a healthy tissue or cell (i.e., that is negative for triple negative breast cancer). In another embodiment, the reference is a baseline level of marker present in a biologic sample derived from a patient prior to, during, or after treatment for triple negative breast cancer. In yet another embodiment, the reference is a standardized curve. The level of any one or more of the markers described herein (e.g., a combination of viral, bacterial, fungal, helminth, and/or protozoan biomarkers) is used, alone or in combination with other standard methods, to characterize the disease, disorder, or condition (e.g., triple negative breast cancer).

In certain embodiments, one or more pathogenic organisms described herein may be isolated or extracted from a sample using a capture reagent (e.g., an antibody) and/or detected using ELISA. In a particular embodiment, reagents for capturing the pathogenic organism include streptavidin bound magnetic beads and biotin labelled probes. Such techniques can be further used to obtain nucleic acids pathogenic organism detection using nucleic acid based probes or for direct sequencing (e.g., miSeq; Illumin).

### Kits

The invention provides kits for the detection of a biomarker, which is indicative of the presence of one or more biological sequences or agents associated with triple negative breast cancer capable. The kits may be used for detecting the presence of multiple biological agents associated with triple negative breast cancer. The kits may be used for the diagnosis or detection of triple negative breast cancer. In some embodiments, the kit comprises a panel or collection of probes to nucleic acid biomarkers (e.g., PathoChip) delineated herein as specific for detection of triple negative breast cancer. In additional or alternative embodiments, the kit comprises an antibody specific for a pathogenic organism associated with triple negative breast cancer. Such antibodies may be used for ELISA detection or for extraction of a pathogenic organism associated with triple negative breast cancer (e.g., a biotin labelled antibody in conjunction with streptavidin bound magnetic beads).

In some embodiments, the kit comprises one or more sterile containers which contain the panel of probes, nucleic acid biomarkers, or microarray chip. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The instructions will generally include information about the use of the composition for the detection or diagnosis of triple negative breast cancer. In other embodiments, the instructions include at least one of the following: description of the therapeutic agent; dosage schedule and administration for treatment or prevention of triple negative breast cancer or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

One embodiment of the invention is a kit comprising at least three nucleic acid probes selected from the group consisting of SEQ ID NOS: 1-160. The kit can include probes from about 10-30 organisms with about 3-5 probes per organism. Another embodiment of the invention is a kit comprising a microarray with at least three nucleic acid probes selected from the group consisting of SEQ ID NOS: 1-160. The kits contain instructional materials for use thereof.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Materials and Methods

### PathoChip Design.

A metagenomic approach for the design of the 60,000 probe sets of selected microorganisms termed the PathoChip Array has been previously described (Baldwin et al. (2014) M Bio 5, e01714-01714). The designed probe sets were manufactured as SurePrint glass slide microarrays (Agilent Technologies Inc.). Probes were represented as 60-nt DNA oligomers with 60,000 probes on 8 replicate arrays per slide. These target pathogenic viral, prokaryotic, and eukaryotic genomes with multiple probes for each organism is combined with upstream sample preparation and amplification protocols to detect DNA and RNA of microorganisms and downstream data analysis. PathoChip screening of DNA plus RNA from formalin-fixed paraffin-embedded (FFPE) tumor tissues has been established, and the detection of oncogenic viruses was previously validated (Baldwin et al. (2014) M Bio 5, e01714-01714). Previous studies demonstrated the use of the PathoChip technology, combined with PCR and HT sequencing, as a valuable strategy for detecting the presence of pathogens in human cancers and other diseases (Baldwin et al. (2014) M Bio 5, e01714-01714).

### Sample Preparation and Microarray Processing.

De-identified formalin-fixed paraffin-embedded (FFPE) triple negative breast cancer samples (n=100) were received from the Abramson Cancer Center Tumor Tissue and Biosample Core in the form of 10 μm sections on non-charged glass slides and matched (n=20) control samples and non-matched (n=20) control samples were provided as paraffin rolls. Matched controls were obtained from adjacent non-cancerous breast tissue of the same patient from which the cancer tissues were obtained. Non-matched controls were breast tissues obtained from healthy individuals. The rolls or mounted sections (5 sections per sample) from FFPE samples were used for parallel DNA and RNA extraction) as previously described (Baldwin et al. (2014) M Bio 5, e01714-01714). The quality of the extracted DNA/RNA was assessed by measuring the A_{260/280} ratio. The size distributions of the extracted nucleic acids were determined by agarose gel electrophoresis. The extracted RNA and DNA samples were partially degraded as expected and were subjected to RNA/DNA amplification as previously described (Baldwin et al. (2014) M Bio 5, e01714-01714) using RNA and DNA (50 ng each) as input. Of the 100 triple negative breast cancer samples screened, 40 were screened individually and 60 were screened in pools of 5 samples (10 ng each of RNA/DNA) per reaction, so a total of 52 arrays were used to screen the 100 triple negative cancer samples. From the 20 matched and 20 non-matched controls, pools of 5 samples (lOng each of RNA/DNA) were used per reaction, for 4 arrays each for screening the matched and non-matched controls. The amplification products were checked by agarose gel electrophoresis, and as expected the size of the amplicon ranged from 200-400 bp for FFPE samples. Human reference RNA and DNA (15ng each) extracted from the BJAB human B cell line was also subjected to WTA. The amplified products were purified using a PCR purification kit (Qiagen, Germantown, Md., USA), and amplified product (2 μg) from the FFPE cancer tissues was used for Cy3 labeling (SureTag labeling kit, Agilent Technologies, Santa Clara, Calif.) and Cy5 labeling was performed on human reference cDNA/DNA amplification product (2 μg) as a control to determine cross-hybridization of probes to human DNA. The labelled DNA was purified and the extent of labeling was determined by A₅₅₀ for Cy3 and A₆₅₀ for Cy5. The labelled samples were hybridized to the PathoChip using conventional methods (e.g., as described by AgilentTechnologies, Santa Clara, Calif.). Hybridization cocktail containing a CGH blocking agent, in hybridization buffer (as per manufacturer's instruction), was added to the labeled test sample (Cy3) and the reference (Cy5), denatured and hybridized to the 8× arrays (PathoChip is a glass slide containing 8 arrays) in a 8-chamber gasket slides at 65° C. with rotation in an Agilent hybridization oven. Post-hybridization, the slides were washed using wash buffer and scanned using an Agilent SureScan G4900DA array scanner.

### Statistical Analysis of PathoChip Data.

Data analysis was done using the Partek Genomics Suite (Partek Inc., St. Louis, Mo., USA) as previously described (Baldwin et al. (2014) M Bio 5, e01714-01714). Model-based analysis of tiling arrays (MAT) which utilized a sliding window analysis of probe signals for each tumor; analysis at the individual probe level (both for specific and conserved probes) and at the accession level (taking account of all the probes per accession) were performed. While the outlier analysis at the individual (specific probe outlier and conserved probe outlier), or at the accession level (accession outlier) revealed probes that show higher hybridization signal in some samples, the paired t-tests with False Discovery Rate (FDR) multiple correction at the individual probe (specific probe t-test, conserved probe t-test) or at the accession level (accession t-test) revealed the probes that are significantly detected across the 100 tumor samples analyzed. Two-sample Wilcoxon tests were performed to determine if cancer samples had significant detection of the candidate signature of organisms compared to the control (both matched and non-matched) samples. Hierarchial clustering of the samples based on the detection of pathogenic signatures was done using the R program (Euclidean distance, complete linkage, non-adjusted values).

### PCR Validation of PathoChip Results.

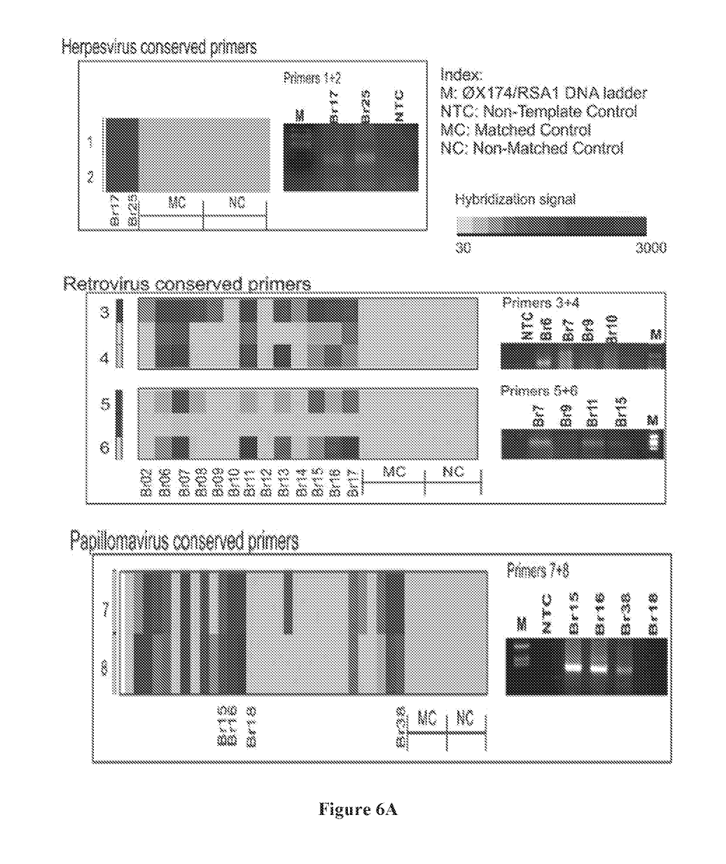

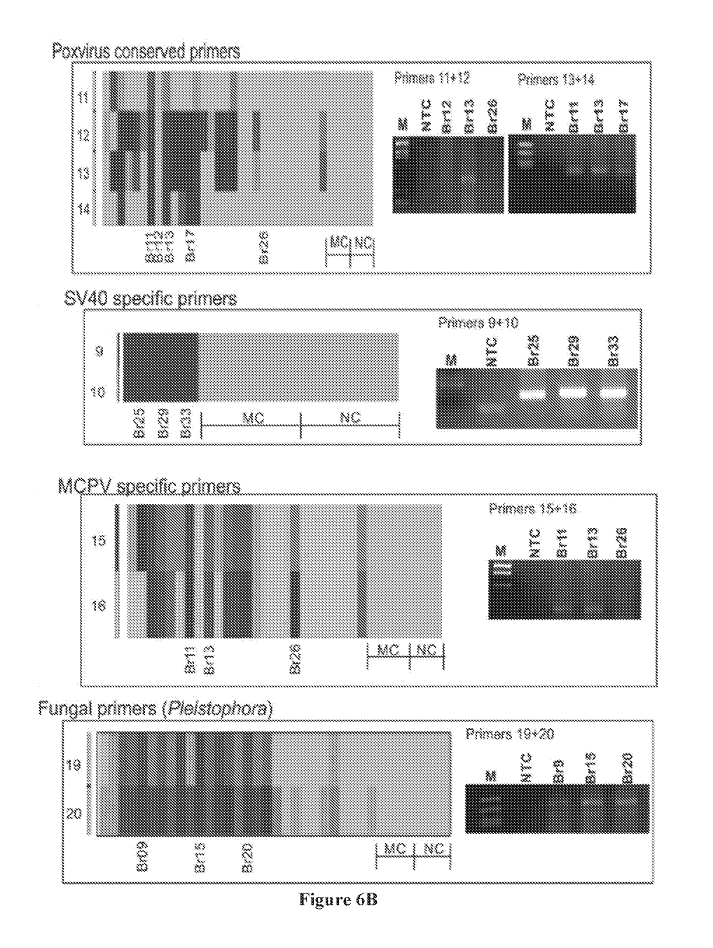

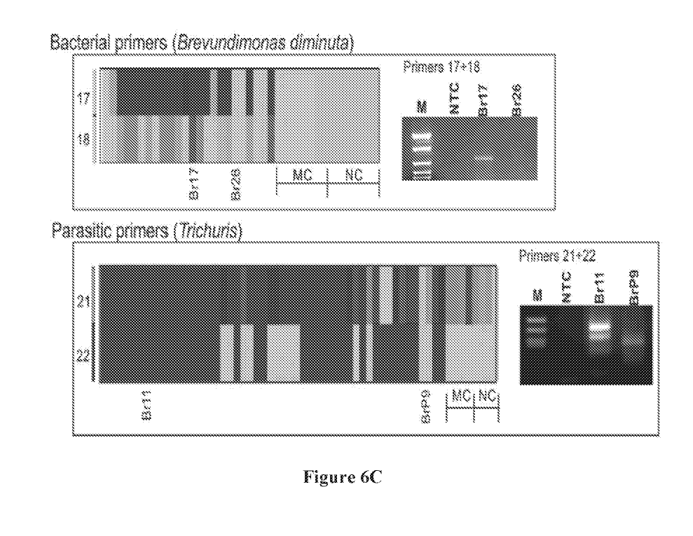

PCR primers were designed from the conserved and specific probes of organisms with hybridization signals that represent a signature pattern. The PCR amplification reaction mixtures for each reaction contained 200 ng of tumor DNA and 10 pmol each of forward and reverse primers (Table 1), 300 μM of dNTPs and 2.5U of LongAmpTaq DNA polymerase. DNA was denatured at 94° C. for 5 min, followed by 30 cycles of 94° C. for 30 sec., 48-57° C. for 30 sec., and 65° C. for 20-60 sec. The annealing temperature was different for different sets of primers used, mostly 5 degrees below the melting temperature of the forward and reverse primers for each set of primers. The PCR conditions for each of the primer set are provided in Table 1. Validation of the PathoChip hybridization results by PCR is presented in FIGS. 6A-6C.

TABLE 1
Primers used for PCR validation of PathoChip screening.

| | | | Annealing | Extension | |
|---|---|---|---|---|---|
| | | | temp and | temp and | Amplicon |
| | Primers | Sequence (5′-3′) | time | time | size (bp) |
| Herpes | FP 1 | GAA GAC GCT GAT GAA CCA CG | 51° C. for | 65° C. for | 96 |
| | RP 2 | AAG CAC CTG GTG TAC TTT CAC | 45s | 20s | |
| MMTV | FP 3 (Env) | TTA GGG GAG AAG CAG CCA AGG | 55° C. for | 65° C. for | 184 |
| | SN RP 4 (Env) | AAA GAG TCA AGG GTG AGA GCC | 30s | 30s | |
| | RP (Env) | CTT GTA AGA GGA AGT TGG CTG TGG | | | |
| MMTV | FP (gag) | CAC AGA TTG GAA CGA TGA TGA CCT G | 57° C. for | 65° C. for | 70 |
| | SN FP 5 (gag) | ACT CAG AAG GAA ACC CCT GCC TC | 30s | 30s | |
| | RP 6 (gag) | ATC TCC TTT TTC CCT GGC CTC TGC | | | |
| Papilloma | FP 7 | CTT GAC ATT GTG TGT CCT GCC TG | 53° C. | 65° C. for | 160 |
| | RP 8 | TAA TTC AAA GGT GTC TGC CTC CTG C | for 30s | 30s | |
| SV40 | FP 9 | CAG TAG CCT CAT CAT CAC TAG ATG | 51° C. for | 65° C. for | 94 |
| | RP 10 | GGA ACT GAT GAA TGG GAG CAG T | 45s | 20s | |
| Parapox | FP 11 (orf) | ATC TTC ACG GGC GCA GTC G | 56° C. for | 65° C. for | 286 |
| | RP 12 (orf) | CTC TTC GAC GAC GAC GGG AAC | 30s | 30s | |
| | FP 13 (PcP) | TCGTGATCTCGGTGTCCACCTG | 56° C. for | 65° C. for | 524 |
| | RP 14 (PcP) | CAT CAA CTA CCT GCT CGA CAG CAC | 30s | 30s | |
| MCPV | FP 15 | CAG AGA GGA GAC CAC CAA TTC AG | 52° C. for | 65° C. for | 264 |
| | RP 16 | GTG AAG GAG GAG GAT ATG TAT TCC | 45s | 30s | |
| Bacteria | FP 17 | TTG CAG AGG ACA ATC CGA ACT GAG | 52° C. for | 65° C. for | 667 |
| | RP 18 | AAC TGC CTT TGA TAC TGG CGA TC | 60s | 60s | |
| Fungus | FP 19 | AGG TCT CCT AGG TGA ATA GCC | 48° C. for | 65° C. for | 219 |
| | RP 20 | CCG TGC TTA CAG TTA TTT CCT C | 30s | 30s | |
| Parasite | FP21 | GAG GTA GTG ACG AAA AAT AAC GG | 48° C. for | 65° C. for | 250 |
| | FP22 | CCA GAG TCT CGT TCG ATA TCG | 30s | 30s | |

### Probe Capture and High-Throughput Sequencing.

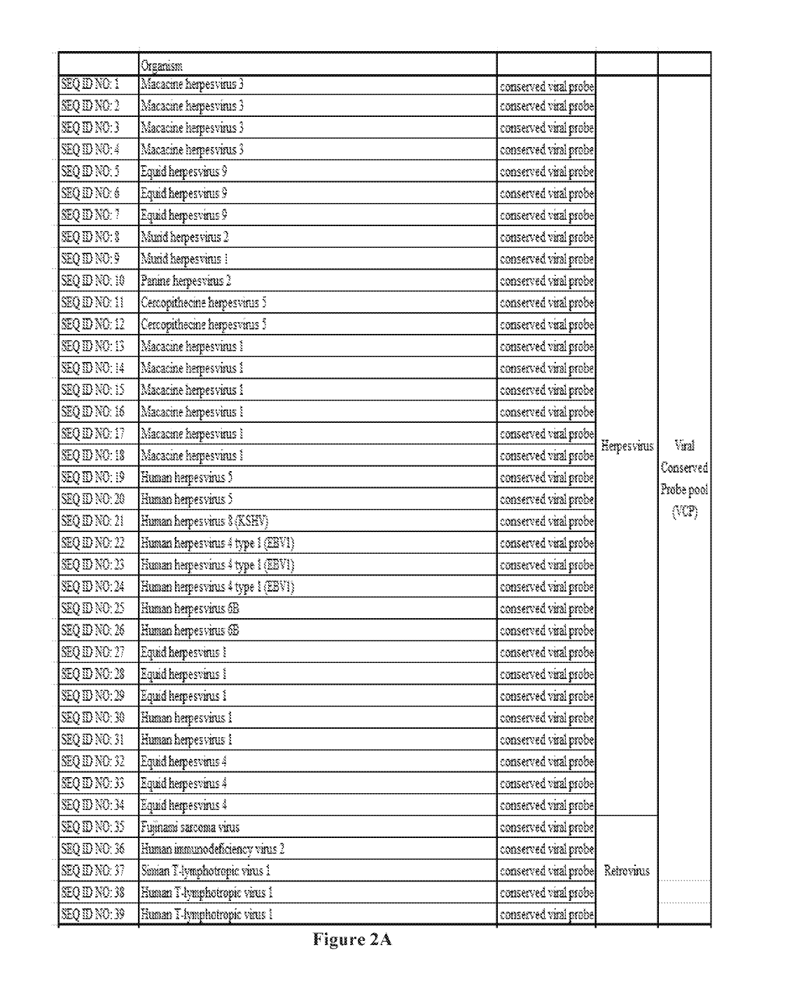

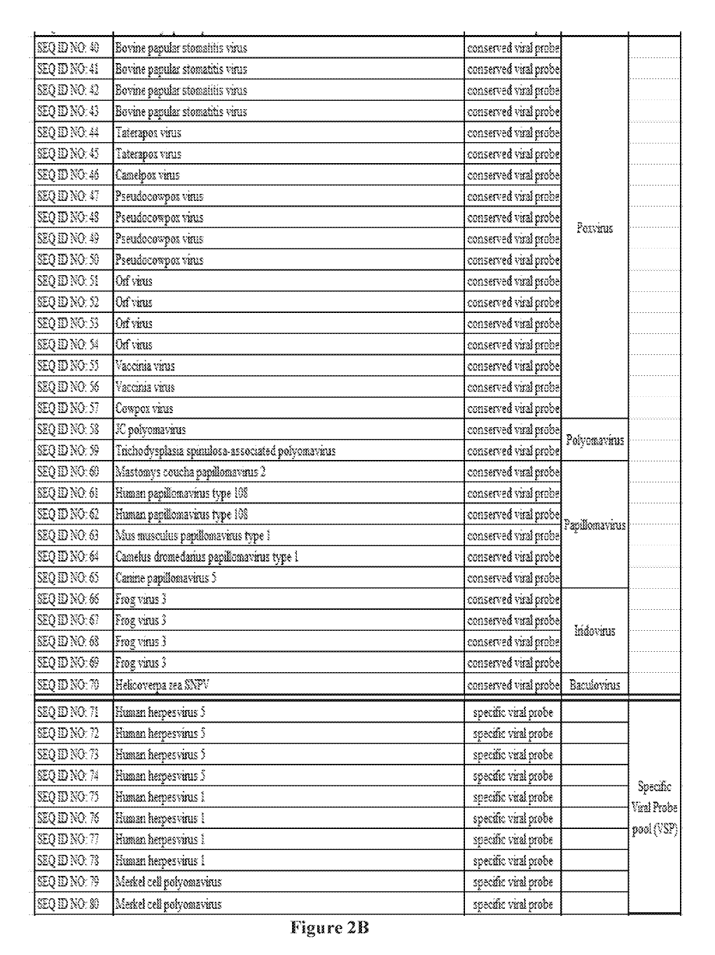

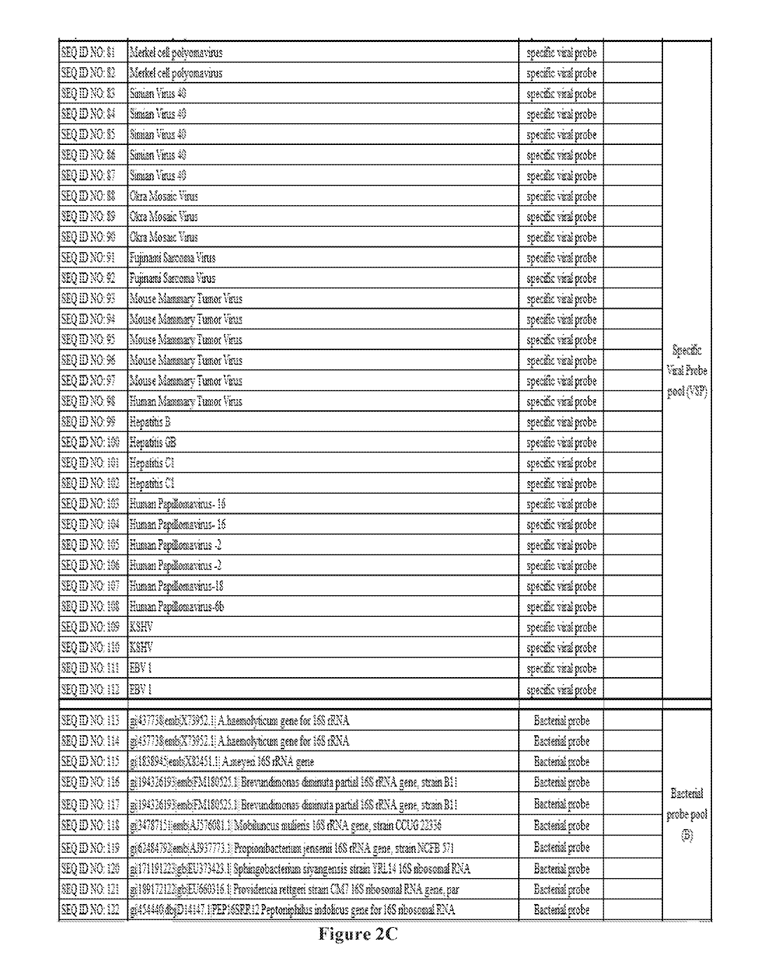

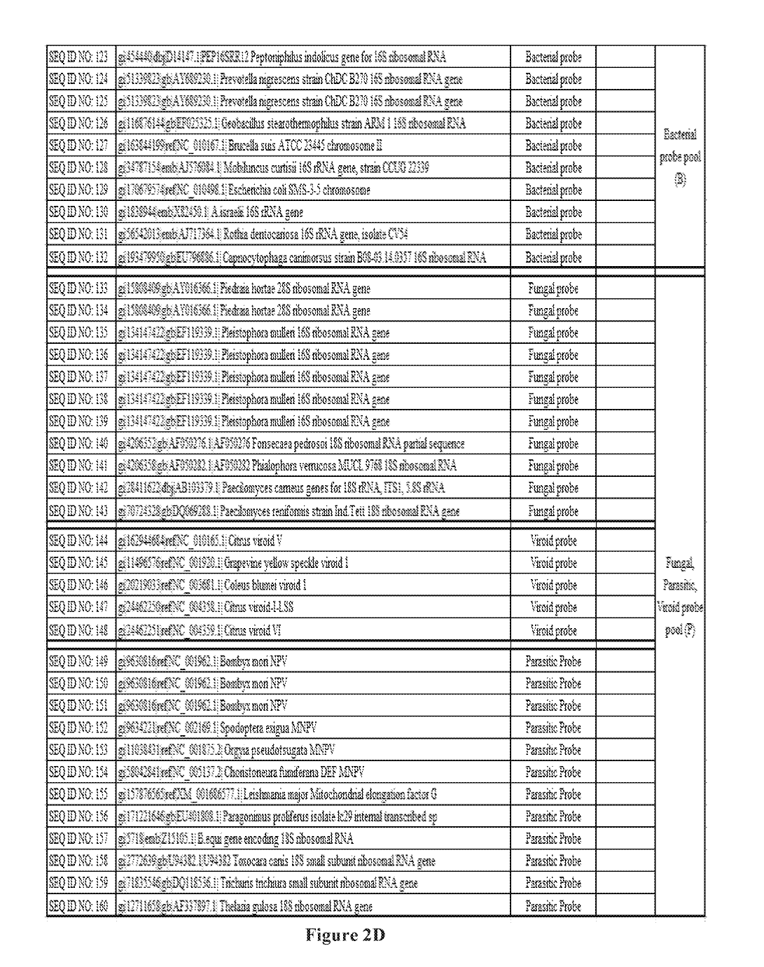

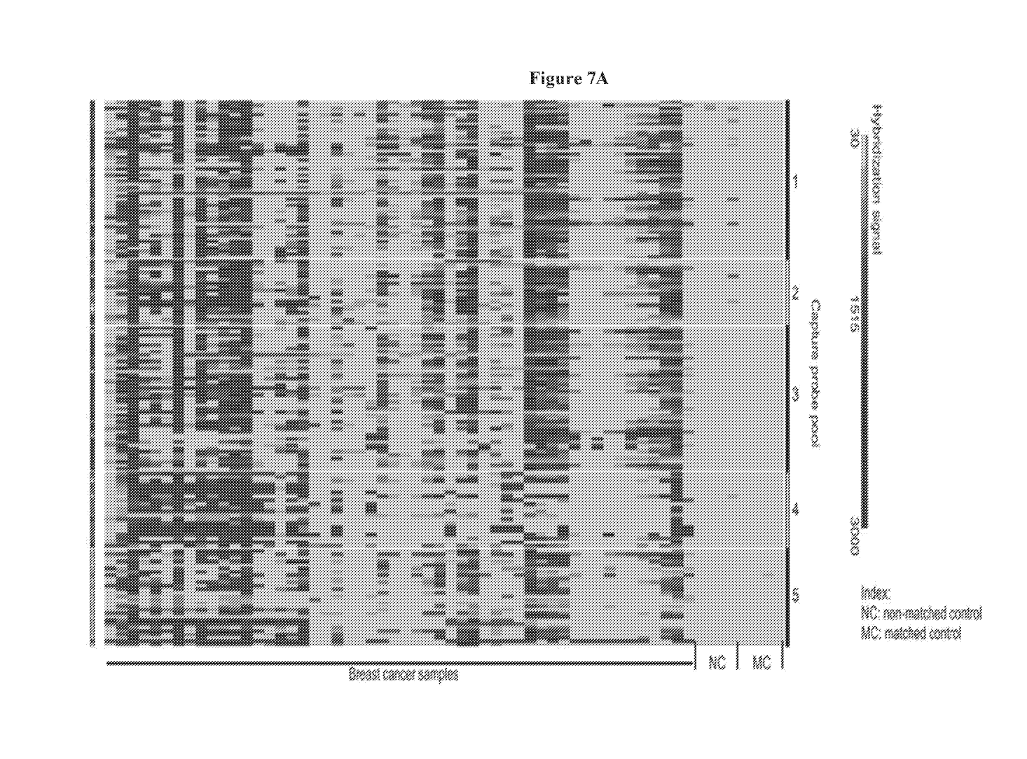

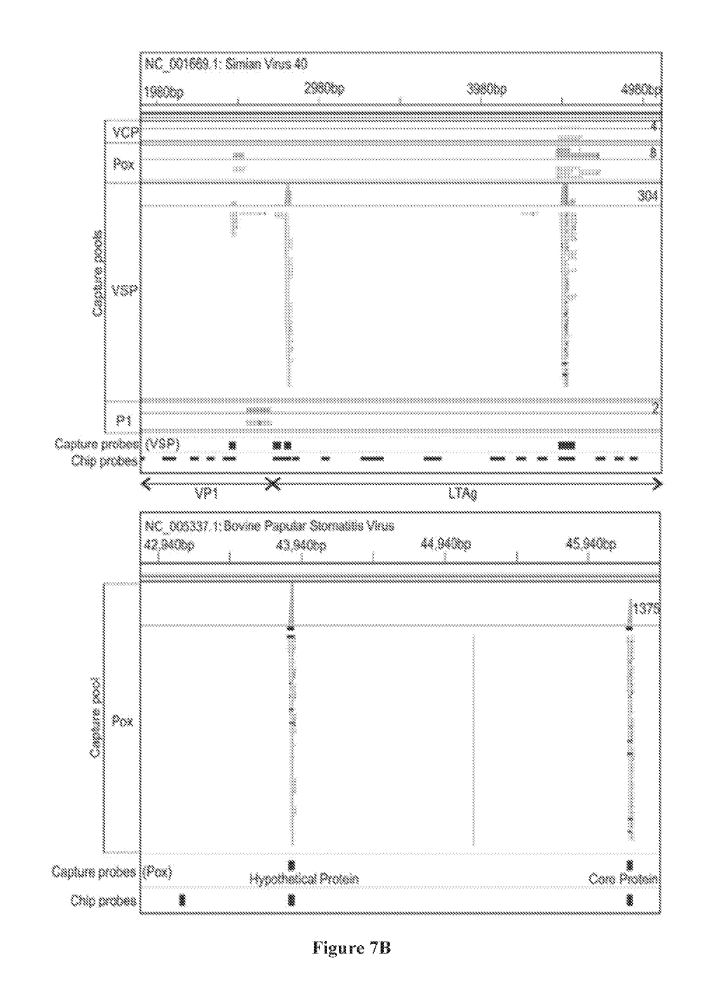

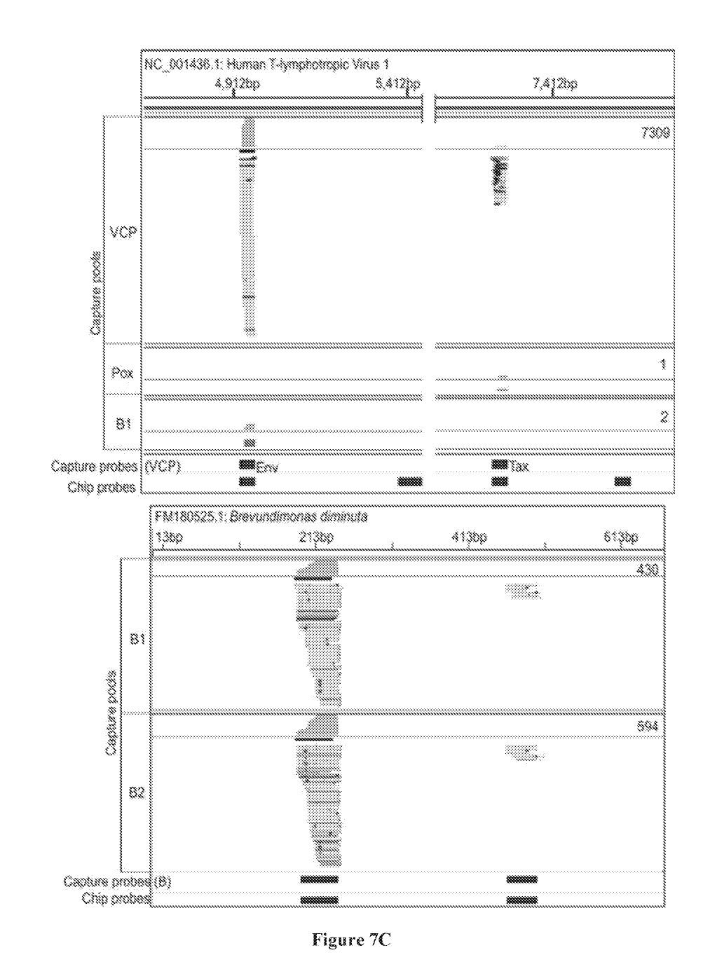

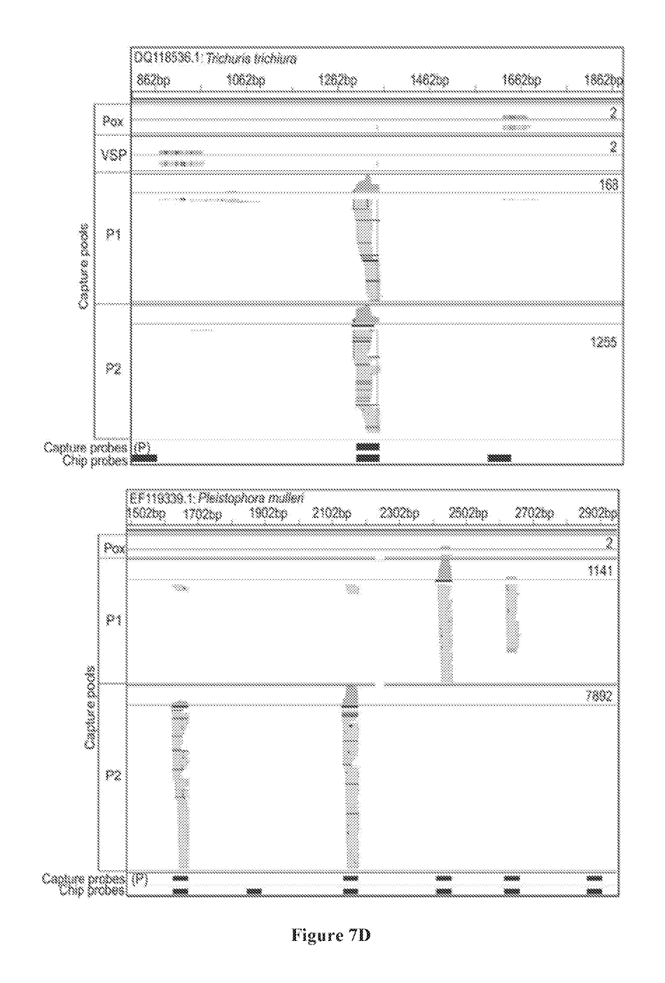

Libraries of targeted sequences were captured by magnetic beads to generate libraries for high throughput sequencing. Selected PathoChip probes with high hybridization signals in triple negative breast cancer samples only were synthesized as 5′-biotinylated DNA oligomers (Integrated DNA Technologies, Coralville, Iowa, USA), mixed as 5 capture probe pools (pools 1-5) (FIGS. 7A-7D, Table 2, FIGS. 2A-2D), and hybridized to pools of tumor samples. Pool 1 contained 52 selected viral conserved probes (VCPs) excluding the pox viral conserved probes; pool 2 contained 18 conserved pox viral probes (Pox); pool 3 contained 43 viral specific probes (VSPs); Pool 4 included 20 selected bacterial probes (B) and Pool 5 contained 28 fungal, parasitic probes (P). Targets were captured by pooling all 100 WTA products used for PathoChip screening (for VCP, Pox, VSP capture) or by pooling 100 WTA samples in two groups (group 1 comprising pool of 18 WTA samples that showed high hybridization signal to B and P probes and group 2 comprising the remaining WTA samples. Each capture probe pool was added to each target pool in reaction mixtures containing 3M tetra-methyl ammonium chloride, 0.1% Sarkosyl, 50 mM Tris-HCl, 4 mM EDTA, pH 8.0 (1XTMAC buffer). Seven (7) individual target captures were done: VCP, Pox, VSP, B1, B2, P1 and P2. The reaction mixtures were denatured (100° C. for 10 minutes) followed by a hybridization step (60° C. for 3 hours). Streptavidin Dynabeads (Life Technologies, Carlsbad, Calif., USA) were added with continuous mixing at room temperature, followed by three washes of the captured bead-probe-target complexes in 0.30 M NaCl plus 0.030 M sodium citrate buffer (2×SSC) and three washes with 0.1×SSC. Captured single-stranded target DNA was eluted in Tris-EDTA (1E) for library preparation and next-generation sequencing.

TABLE 2
Probes used for target capture

| SEQ ID NO | Probe sequence 5′-3′ |
|---|---|
| SEQ ID NO: 1 | TTTCTCGCTCTCACCCTTAACCCGCTGGCGCGCCTGCACCATCTT |
| SEQ ID NO: 2 | CCCGCACTGACACCACACGTCATGCGCCCCCTTGATTTGCAGTCT |
| SEQ ID NO: 3 | GATGAATTTACAGACGCACACCGGAATGCATAAGCAACCAAACGGGATATAAAG |
| SEQ ID NO: 4 | ACCATGAACAAAACTACAGGAATCAAGAACAAAACGGAAGGAGCAGGATCTAC |
| SEQ ID NO: 5 | CAAAAACACGGCAGGAGGGGCCTTTTTCCACGAGTAAGACTCCAT |
| SEQ ID NO: 6 | CTTCTAAACTGTCGTTTGATGCACTAGACGCACCCCCGACTCAAATTATAGA |
| SEQ ID NO: 7 | GTAAAACCACCACTCGTTGGCACCCTGCTTCACCGCAACTCCCAA |
| SEQ ID NO: 8 | GCGGCCCTCCTCGCCGCCCAAGAAGGCCACGGGGATCTCCTTGTA |
| SEQ ID NO: 9 | CTATATAGCAGGAGAGGGAGACCCGACAGCCGGTGTTTTTGAACA |
| SEQ ID NO: 10 | GCAGCGCGTGGCCCTGCCAGTCGCCGCAGTCGCACCACACGTCGT |
| SEQ ID NO: 11 | CTTTGTCTCCAAGGGGACCCCGCGCCGCGCCGTCTGCTACATCAT |
| SEQ ID NO: 12 | CTTAAACGGACAGCCCCTGGGAGAAACCTCCTACTACGGCGGTTG |
| SEQ ID NO: 13 | AAACCCCTCGAGCCGATCCTCGTCCGTGTCGCTGTTCCAGAACCA |
| SEQ ID NO: 14 | ACCAGGAAGGACCAGGCAAACACCAACGCCCGCTTCGAGAACACG |
| SEQ ID NO: 15 | GCGAGGAGCAGCAGGATCAGGTCGGCGTGTCCCCACGCGTCCGCG |
| SEQ ID NO: 16 | CTGCACGAAGAGGATCGCCCCGGCGCCCGTCTCCCACGCCGCGGG |
| SEQ ID NO: 17 | GAGATCGTGCCCTCGACGCCCGCCATGCTGGGCCTGGGGACCCGC |
| SEQ ID NO: 18 | CTGCGTCACCTGCCGGCGCGCGCGGGCGTGGCGGGCCGTTAAAAG |
| SEQ ID NO: 19 | GAAGACGCTGATGAACCACGAGGGCGAGGTGGGGCAGAGGAAGAC |
| SEQ ID NO: 20 | CTGGATCTGCTCCTCCAGGCACTTGATGACCTGCTTCTTAAACAG |
| SEQ ID NO: 21 | GCTCCTGGCAAACTATGTCACCAGGCTCCCCAACCAGAGAAACGC |
| SEQ ID NO: 22 | TATTTGCAAAGGGAGGCGAGGAGATGGAGTGACTGAAGGAGCGATA |
| SEQ ID NO: 23 | ATCTCTGCCGCCATCCCGGCCAGGAAGGCCTCGATGACCGAGTCT |
| SEQ ID NO: 24 | CAACCTCTGCTCCCCTCTATTCTCCTCTTGCGTTATCTCCAATAGAATTTG |
| SEQ ID NO: 25 | GAACAGACCGACTCCGGGCGCGAGGAGGACGCACAGGAGAGCGAG |
| SEQ ID NO: 26 | CGTCCACCGTCCCTCTCACCCCCACTCGAATCGCGCAGGCGCGTC |
| SEQ ID NO: 27 | GGCAAGCACCTCGTTTATTGGGACCGGGGCTGTCCGGCGTCTATT |
| SEQ ID NO: 28 | CAATCAGTGCGCCCGATCTCCCGGCCACTGAACCACAACGGCATG |
| SEQ ID NO: 29 | AGCACAACGCAGACTCCGCCTAGACTCCCGCCTCCATCCGCTGAC |
| SEQ ID NO: 30 | ATAGGCCAGAGCCACTTCCAGAAGCGCAGCAAGATAAAGGTGAAC |
| SEQ ID NO: 31 | CAAACACAACGTGACCCCCCGGGAGACCGTCCTGGATGGCGATAC |
| SEQ ID NO: 32 | ATAATAAAAACGATAACACAGAAGACCCCACACACCTTGTTGCATCTAGGCTGC |
| SEQ ID NO: 33 | ATTTTATCCAACCGGCACCAAACAGGGTAGACTTGTTATTCAAAGATATACCCGAAT |
| SEQ ID NO: 34 | CTACACGGTGGACACCCGGGCCGGAGAGCGCACCCGCGTTCCACT |
| SEQ ID NO: 35 | CACAGGCGGCGTGGCGATCCTGCCCTCATCCGTCTCGCTTAATCG |
| SEQ ID NO: 36 | AAACAAGCAGACATGATGATGAGCATGGGGAGACATTAGTGTGGCAGTTT |
| SEQ ID NO: 37 | CAGAAACTACTACAGGCCCGAGGACACACTAATAGCCCTCTAGGAGATAT |
| SEQ ID NO: 38 | CATACCACTCTAAACCCTGCAATCCTGCCCAGCCAGTTTGTTCAT |
| SEQ ID NO: 39 | CCAACATTCCACCCTCCTTCCTCCAGGCCATGCGCAAATACTCCC |
| SEQ ID NO: 40 | GTCATGGCCCGGCGCTGCGCCCGCAGCAGCACGCACCGCTCCATG |
| SEQ ID NO: 41 | GACGTGGTGCGGTCGCTCATCACCTCCACGCTGCAGCGGGCCGGC |
| SEQ ID NO: 42 | GTTCTTCCGGAAGACGACCCGCTCCACGGCGTCCACCATGTCCAC |
| SEQ ID NO: 43 | CTGCTCCGGCACTCCACCGAGCGCCGCCACCTATTCGTCGACTTC |
| SEQ ID NO: 44 | TAATATCTTCTGGAAGGTTTGTATTCTGAATGGATCCACCATCTGCCATAATCCTATTCT |
| SEQ ID NO: 45 | TAAAGACACTCCACATGCCGTCACTACCTCCGTTAGAAGACATATTAATAAGACTTAAGA |
| SEQ ID NO: 46 | TAATAGAGGAAATCCCACCGCCTTTCTGGATCTCACCAACGACGATA |
| SEQ ID NO: 47 | GATGATGCCCTTGGCCTCGCGGTCGAAGACGGCCACCTCGCTCAC |
| SEQ ID NO: 48 | AGACACTTGAAGTCGACGCCGGACTCGCCGCGCAGCACCGAGCGC |
| SEQ ID NO: 49 | TATGGATTCGGCTATCCAGTCCTTGACCGAGCCCACGATGCCCGC |
| SEQ ID NO: 50 | GTCCGCGTAGCCCGCGCCCACGGCCTTGCCGCAGTCCGCGATCAT |
| SEQ ID NO: 51 | GAAGAGTTTTCACAAAAAGTTTTCGGGAGGAGAGGCTGACCTACCTTC |
| SEQ ID NO: 52 | GGCGGGAGGGAGGGGTCTCGACTGCGGGCGGTCCTTTTTCACTTT |
| SEQ ID NO: 53 | GATCAAGAACAAGACGCGCGTGCCCTTCCTGCTGCTCTCGGCCTC |
| SEQ ID NO: 54 | AACGACCCTGGCTACCACTCGCGGGAGACTCTCTGCAGCGGACCT |
| SEQ ID NO: 55 | TCTTTCTCTTCTTCGCTACATCTGATGTCGATAGACACCTCACAGTCTTTGATCATAG |
| SEQ ID NO: 56 | CTATCAATAACTGGCACAACAATAACAGGAGTTTTCGCCGCCGCCATTTAGTTATT |
| SEQ ID NO: 57 | ATTACGAAGAAGACGACGAGGACGGAGACGGTAGAATAAGTGTAGCAAATAAAATCTATA |
| SEQ ID NO: 58 | TAACAGCCAGTAAACAAAGCACAAGGGGAAGTGGAAAGCAGCCAA |
| SEQ ID NO: 59 | CGTCCGGTCTCCATAACAACACATCCTCCCGCTCTGTGTTCTCAC |
| SEQ ID NO: 60 | TTAGACTCTACAAAAGGCAGGAGATGAGGGACATGACAATGGCTCAGT |
| SEQ ID NO: 61 | CTTGACATTGTGTGTCCTGCCTGTGCCAAGCAACGAGAACGAAAT |
| SEQ ID NO: 62 | GTTAAAGAAGCAAACTATGTTAAACCACCAGCAGGAGGCAGACACCTTTGAATTA |
| SEQ ID NO: 63 | ATGAGACAGAGGAAGAAGGGGACTGGAAGGTTATTGCAAACTTCCTTAGATA |
| SEQ ID NO: 64 | ATATGATGGAAATTGGGTTTGGGGCTGCAAATTTCAAGGCCTTAAATCAGTCTAAATC |
| SEQ ID NO: 65 | ATAGATGAGGAAGGGGACTGGAAGCACATAGGGAACTTTCTTAGATTCCA |
| SEQ ID NO: 66 | GACTGTGGAGGAGGGTGCAGGATAGAGTCTGGAAAGATTGTCTCT |
| SEQ ID NO: 67 | GCACTCCTTGAGCCTCTCCCCCTTGACCCTCATCTTCTTGACAAG |
| SEQ ID NO: 68 | AGATCTCTCCGGGTGGCTCCTGTTGACCGGGGTGGCCGTCCAGTT |
| SEQ ID NO: 69 | ATCAAGATGAGCAAGATTGGAAAGGGCTGCACCCTCGTCATGGCG |
| SEQ ID NO: 70 | TTTCCATAGACGACGTGGACGCGTTTGTGTCTGTTTTGACGGTTTTTAAA |
| SEQ ID NO: 71 | ACATCCATGGCTCGCCGTCTGCTTCTCTGCCGCTCGTGGTGCCGA |
| SEQ ID NO: 72 | GGACGCTGCTACAACCACCGTGTCGTCCGCGTTCGTCGTCCCCAG |
| SEQ ID NO: 73 | GTCTCGCGGCGGCTCCCTCTCGGCGGCTCCGGTTGGGCTCCCCTC |
| SEQ ID NO: 74 | GACCACATCCCGCTCCTGCTCATCGTCACGCCCGTGGTCTTTGAC |
| SEQ ID NO: 75 | AAAGGGGTTGGACATGAAGGAGGACACGCCCGACACGGCCGATAC |
| SEQ ID NO: 76 | ATCCCCTCGAAGAACGCGCCCAGGCCCGCAAACATGGCGGCGTTG |
| SEQ ID NO: 77 | GACCCCAGGCGTGCCGGGGGAACTCGGAGCCGCCGACGCCACCAG |
| SEQ ID NO: 78 | CGGAGTGGCAGGGCCCCCGTTCGCCGCCTGGGTCGCGGCCGCGAC |
| SEQ ID NO: 79 | ATATACCTCCCGAACACCATGAGGAACCCACCTCATCCTCTGGAT |
| SEQ ID NO: 80 | TCTGGATCCAGTAGCAGAGAGGAGACCACCAATTCAGGAAGAGAAT |
| SEQ ID NO: 81 | GTTTACAGATTAGGAATACATATCCTCCTCCTTCACCACCCCGAAGACC |
| SEQ ID NO: 82 | GAATATGGGCCCAATCCACACGGGGCCAACTCAAGATCCAGAAAG |
| SEQ ID NO: 83 | TATGATCATGAACAGACTGTGAGGACTGAGGGGCCTGAAATGAGC |
| SEQ ID NO: 84 | TAATTAACAGGAGGACACAGAGGGTGGATGGGCAGCCTATGATTG |
| SEQ ID NO: 85 | AGCAGTAGCCTCATCATCACTAGATGGCATTTCTTCTGAGCAAAACAGGTTTTC |
| SEQ ID NO: 86 | TTCAGGGGGAGGTGTGGGAGGTTTTTTAAAGCAAGTAAAACCTCTACAAAT |
| SEQ ID NO: 87 | TTTTCCTCATTAAAGGCATTCCACCACTGCTCCCATTCATCAGTTCCATAG |
| SEQ ID NO: 88 | AACGCGTCACCTCATCCGCCCGATGGCTATCCAAAACCGCCACCT |
| SEQ ID NO: 89 | CTTCGGTCCAAACAACTCACCTGCTCCGAAATCCGAATCTTCCAA |
| SEQ ID NO: 90 | TTCAACACCTCCTCCGAACTCGCCCCTTTTCCTCCTTCCGCGTCT |
| SEQ ID NO: 91 | GAGAAACCAGCAACGGAGCGGCGAATCGACAAGGGAGAAACAACT |
| SEQ ID NO: 92 | CTCATCGACCACCTGCTGCAGAGCCAGCGGCCCATCACCCGCAAG |
| SEQ ID NO: 93 | CGTGAGTTAGGTCGAGCAGAGCCAAAGCCCCCGGTGCTTCGTCGC |
| SEQ ID NO: 94 | TTGCCTTGCGCCTTCCCTGACCAGGGGGTGAGTTTTTCTCCAAAA |
| SEQ ID NO: 95 | GAGAGTGTCCTACACTTAGGGGAGAAGCAGCCAAGGGGTTGTTTC |
| SEQ ID NO: 96 | ACCTTCCTCCTGAGGCAAGGACCACAGCCAACTTCCTCTTACAAG |
| SEQ ID NO: 97 | CAGGAGCGATGGCAGAGGCCAGGGAAAAAGGAGATTTGACTTTTA |
| SEQ ID NO: 98 | GAAAGATTTTTCATTATACCAAGGAGGGGGCAGTGGCTAGACAATTAGAACACATTTCT |
| SEQ ID NO: 99 | AACAGTAAACCCTGTTCCGACTACTGCCTCACCCATATCGTCAATCTT |
| SEQ ID NO: 100 | GCGCTTTCCACCGGATACTCTGGCAACTTTGACTCAGTTACTGATT |
| SEQ ID NO: 101 | TCTCTTGCCTGACTGTGCCCGCTTCAGCCTACCAAGTGCGCAATT |
| SEQ ID NO: 102 | GCAGGAGATGGGCGGCAACATCACCAGGGTTGAGTCAGAGAACAA |
| SEQ ID NO: 103 | ACCCATACCAGGGTCTCGCCCAGTGGCACGCCTAGGATTATATAG |
| SEQ ID NO: 104 | GAAGAAACACAGACGACTATCCAGCGACCAAGATCAGAGCCAGAC |
| SEQ ID NO: 105 | ACACATCTGCTTGTGCTACTGCTCTTCCTGTGGCTCTCTCAACTAAC |
| SEQ ID NO: 106 | TAGACCTAAACAGTCCAGAGGAGCAGGACGACAATGGAAACACTG |
| SEQ ID NO: 107 | CACCATAGGCCCTCGCAAACGTTCTGCTCCATCTGCCACTACGTC |
| SEQ ID NO: 108 | TTTCCAAAGCCTCTGCTGCCCCTAAACGTAAGCGCGCCAAAACTA |
| SEQ ID NO: 109 | GTCCAAGGCACCCTGGGTCCTCTTACGAATGTCTGACTACTTCAG |
| SEQ ID NO: 110 | GTAAGAGGGAGACCCAAAGGCGGCGGCACTAAAGATTGTTCTGGT |
| SEQ ID NO: 111 | TTCTTGAAAAGGACGACCAGCACATGGAGCAGCAGGTTATGGCAA |
| SEQ ID NO: 112 | AGTCATCCCTGTTACAGTCTCCGGGAAGGGCCTTTGCACCCGTTA |
| SEQ ID NO: 113 | GAATCCCTTAAAGCCAGTCTCAGTTCGGATTGGGGTCTGCAACTC |
| SEQ ID NO: 114 | CGTGGCCTAACTCGTTTGAGGGGGAGCGGACGAAGGTGGGATTAG |
| SEQ ID NO: 115 | GAATCCCTTAAAGCCAGTCTCAGTTCGGATTGGGGTCTGCAACTC |
| SEQ ID NO: 116 | GAGTTGCAGAGGACAATCCGAACTGAGACAATTTTAAGGATTAACCCTCTGTAG |
| SEQ ID NO: 117 | AAAGCCACGTCTCCGTGCGGTCCAGGCATGTCAAAAGGTGGTAAG |
| SEQ ID NO: 118 | CAACTCGACCCCATGAAGTTGGAGTCGCTAGTAATCGCAGATCAG |
| SEQ ID NO: 119 | GAATCTCAAAAAGCCAGTCTCAGTTCGGATTGGGGTCTGCAACTC |
| SEQ ID NO: 120 | AATATGATGCTAATCTCTAAAAGCCATTCACAGTTCGGATTGGGGTCTGCAACTC |
| SEQ ID NO: 121 | GGGAACTTCGGTCCTTGCGCTATCGGATGAACCCATATGGGATTA |
| SEQ ID NO: 122 | CCTGAGAGGGTGAACGGCCACATTGGAACTGAGAAACGGTCCAAA |
| SEQ ID NO: 123 | GATAGCAAGCGAATCTCAAAAAGCCTATCTCAGTTCGGATTGTTCTCTGCAACT |
| SEQ ID NO: 124 | CAACGGCCCACCAAGGCGACGATCAGTAGGGGTTCTGAGAGGAAG |
| SEQ ID NO: 125 | GAACCTTACCCGGGCTTGAATTGCAGGTGCTGCCCACAGAGACGT |
| SEQ ID NO: 126 | GAATCCCAAAAAGCCGCTCTCAGTTCGGATTGCAGGCTGCAACTC |
| SEQ ID NO: 127 | GATCCCAGACCCCGGCTTTGCGCCAGCACACGAAGCGGTTGTAAC |
| SEQ ID NO: 128 | CAACTCGACCCCATGAAGTTGGAGTCGCTAGTAATCGCAGATCAG |
| SEQ ID NO: 129 | GGGCGTCTAAGTTACCAATTCTCGTCTGATGGCTACATACGGCGGTCAGTTTACGCTTAC |
| SEQ ID NO: 130 | ATGAAAGCCGGCGACACCCGAAGCCCGTGGCCCTGTGGGGAGCGG |
| SEQ ID NO: 131 | CTAATCCCTAAAAGCCGGTCTCAGTTCGGATTGGGGTCTGCAACT |
| SEQ ID NO: 132 | GTTAAGTCCTATAACGAGCGCAACCCCTGCGAATAGTTGCCATCATTAAGTT |
| SEQ ID NO: 133 | CTTCTTGACCAGGCTCACTTCGCCGCCGACGGGCCAGCATCGCTT |
| SEQ ID NO: 134 | AAACGAAGCCCGGGCGAGTAGGCAGGCGCGGGGGCCGTGACGAAG |
| SEQ ID NO: 135 | CCGCAGAGGGTGATAGCCCCGTAACCGGCGACAGCGAGGGAGTAG |
| SEQ ID NO: 136 | CGAACGAACTGCGAATGAGCCTGGCGCGGCGTGCGTTTTAATGAC |
| SEQ ID NO: 137 | GATGCGCCTCTAGAGGTAGGGGGGCGGACCGATGCTGCAGAAGGC |
| SEQ ID NO: 138 | GATAGAGAAACAGGGGTGTGTTCCTGTCCCGCGCTGCCGTGCGGC |
| SEQ ID NO: 139 | AGGTCTCCTAGGTGAATAGCCTCTGGTTGATGTTGAACGCAGGTAA |
| SEQ ID NO: 140 | CTTAATCTGACCGCCGGAGGACCGCCTAATACGGGTGTTGCCTCT |
| SEQ ID NO: 141 | TTGCTTTGGCGGACCCGTCTCACGACCGCCCTGGGACCGCTGAAA |
| SEQ ID NO: 142 | AAATGACTTGGCGGCCTCGTCGCGGCCCTCCTCTGCGTAGTATAG |
| SEQ ID NO: 143 | AACTTGCTTGCCGCGTCCTCCTCGCGCCCTGCAACCAGGCCTCTC |
| SEQ ID NO: 144 | CTGCTCTAAGATCTTCGCTGCTGAGGCCCGCGCCGCCGCTCTTCC |
| SEQ ID NO: 145 | AAAGAAGAAGATAGGGGCAGAGGGGGAGTGAGCCTCGTCGTCGAC |
| SEQ ID NO: 146 | CAACGGAATCCAGTGCCCACCGGAGCGCCAGTTCGTGCGAGAGTT |
| SEQ ID NO: 147 | CTTCCGTCTCTACCCTCCCGAGGCGCTTTTCTCACTGACCGACTT |
| SEQ ID NO: 148 | ACTCTCACGCCCACCCGCACGGCTGCTCCGAGGGAGGGGCTCTCT |
| SEQ ID NO: 149 | ACGACGACAACGCACAGAAATATTAGTAGTAAACCGGCTGCTCATTGGAAATACTTT |
| SEQ ID NO: 150 | AATTCGGGCGTGTTTTTCACCAAATCCCACATGGCCGGGCTACTA |
| SEQ ID NO: 151 | CGACAACGACAACTCTATGATAATAGACTTGTGTTCCGACGCGCGCATAATC |
| SEQ ID NO: 152 | GTTTGTTTATGATCTTGGAGGCGGACAAGGCGGTGTTGTTGTGTG |
| SEQ ID NO: 153 | TATTTCATCACAACGTTGTTGCACATGAGCAGGCTGGACACGACC |
| SEQ ID NO: 154 | AAACTTTTTTACTGCCGTCTTTGTTACACGCACGCCGACTGGTTGTG |
| SEQ ID NO: 155 | GCGTGGTGACCGAGACCGCTGTAGATGGCCCTGATGCAGTGATCC |
| SEQ ID NO: 156 | CTCGTGGCTGTGGGGTGCCAGATCTGTGGCGTTTCCCTAACATAT |
| SEQ ID NO: 157 | TAACCATAAACGATGCCGACTAGAGATTGGAGGTCGTCAGTTTGAACGA |
| SEQ ID NO: 158 | TAACCCGTTGAAAATCCTCCGTGATCGGGATCGGGAATTGCAATTATTT |
| SEQ ID NO: 159 | CTAATTCCGATATCGAACGAGACTCTGGCCTACTAACTAGCGGCGGTATTA |
| SEQ ID NO: 160 | CTCGCCGGCCCGCCGCCGATGATGATGATGAAGCGACAGCCTCCAACAACAATAATGATA |

The seven captured eluates were re-amplified by GenomePlex reactions (Sigma-Aldrich, St. Louis, Mo.), purified and assessed for size distribution by agarose gel electrophoresis. Sequencing libraries were prepared using Nextera XT sample preparation kit (Illumina, San Diego, Calif., USA), according to manufacturer protocols. The samples were submitted to the Washington University Genome Technology Access Center (St. Louis, Mo.) for quality control measurements, library pooling, and sequencing using an Illumina MiSeq instrument with paired-end 250-nt reads. Pre-processed raw reads were trimmed to remove low-quality ends (Phredscore<30). Reads were aligned against the human reference genome using Bowtie2 (sensitive-local mode) (Langmead et al. (2009) Genome Biol 10, R25). Reads that could be mapped to human genome with high quality were excluded. The remaining reads were aligned to the PathoChip metagenome, using Bowtie2 (sensitive-local mode) (Langmead et al. (2009) Genome Biol 10, R25). The total number of reads from each library, the number of reads mapping to pathogenome versus the human genome are shwoin in Table 6. There were 680,534 reads from the 7 libraries that were aligned to the PathoChip metagenome. The 202,905 reads with mapping quality score MapQ>=20 were used for further visualization and quantification analysis using Integrative Genomics Viewer 2.3.25 (Petropoulos (1997) Retroviral Taxonomy, Protein Structures, Sequences, and Genetic Maps. In: Coffin J M, Hughes S H, Varmus H E, editors. Retroviruses. Cold Spring Harbor (N.Y.): Cold Spring Harbor Laboratory Press).

TABLE 6
Number of reads generated in MiSeq

| | | Trimmed reads | Reads | | | Reads mapped to |
|---|---|---|---|---|---|---|
| | | (removing | mapped to | Reads not | Reads | pathogenome with |
| | Total | low-quality | human | mapped to | mapped to | quality score |
| Libraries | reads | reads) | genome | human | pathogenome | MapQ >=20 |
| VCP | 1041326 | 967810 | 713524 | 254286 | 126826 | 30042 |
| VSP | 1186563 | 1114046 | 913188 | 200858 | 12545 | 7715 |
| Pox | 1203986 | 1143755 | 896208 | 247547 | 19529 | 7265 |
| B1 | 579207 | 542245 | 128343 | 413902 | 164849 | 12813 |
| B2 | 717949 | 671654 | 191078 | 480576 | 193946 | 14051 |
| P1 | 1324969 | 1239586 | 986414 | 253172 | 21388 | 10532 |
| P2 | 689316 | 646007 | 208228 | 437779 | 141451 | 120487 |
| Total | 6743316 | 6325103 | 4036983 | 2288120 | 680534 | 202905 |

### Results

### PathoChIP Screening of Triple Negative Breast Cancer Samples Detected Signatures of Viruses and Other Pathogenic Organisms.

TNBC samples (n=100) were screened along with matched (n=17), and non-matched controls (n=20) using the PathoChip. All samples were derived from formalin-fixed paraffin embedded archival samples (see Materials and Methods above). Of the 100 TNBC samples screened, 40 were screened individually and 60 were screened in pools of 5 samples (10 ng each of RNA/DNA) per reaction, for a total of 52 arrays used to screen the 100 triple negative cancer samples. From the 17 matched and 20 non-matched controls, samples were pooled to have for 4 arrays each for screening the matched and non-matched controls. Normalized signals which were positive in the controls were then compared to the test samples to determine the probes that were unique to the test samples with significantly higher signals. The results detected viral conserved and specific probes, as well as bacterial, fungal and parasitic probes in the cancer samples (FIGS. 4A-4D; Tables 3-4).

TABLE 3
Number of viral and microbiomic probe signatures detected by screening 100
triple negative breast samples by the PathoChip.
A. Number of viral Probe signatures detected by individial probe analysis.

| | Retroviridae | | | Polyomaviridae | | | Herpesviridae | | |
|---|---|---|---|---|---|---|---|---|---|
| | MMTV | HTLV-1 | FSV | SV40 | JC | MCPV | HCMV | EBV | KSHV |
| Total Probes | 31 | 41 | 8 | 41 | 42 | 62 | 299 | 235 | 259 |
| Specific | 31 | 37 | 5 | 41 | 40 | 62 | 275 | 149 | 256 |
| t-test | 1 | 4 | 4 | 25 | 12 | 27 | 139 | 61 | 132 |
| Outlier test | 30 | 0 | 0 | 0 | 0 | 0 | 1 | 0 | 0 |
| Conserved | 0 | 4 | 3 | 0 | 2 | 0 | 24 | 86 | 3 |
| t-test | 0 | 3 | 2 | 0 | 1 | 0 | 15 | 79 | 3 |
| Outlier test | 0 | 0 | 0 | 0 | 0 | 0 | 0 | 0 | 0 |

| | Papillomaviridae | | Hepadnaviridae | | Parapoxviridae | | | |
|---|---|---|---|---|---|---|---|---|
| | HPV16 | HPV6b | HBV | HCV-1 | BPSV | PCP | Tatera | Orf |
| Total Probes | 68 | 91 | 49 | 121 | 109 | 105 | 200 | 111 |
| Specific | 67 | 90 | 47 | 119 | 12 | 12 | 10 | 13 |
| t-test | 19 | 37 | 25 | 72 | 1 | 3 | 0 | 1 |
| Outlier test | 0 | 0 | 0 | 0 | 0 | 1 | 0 | 0 |
| Conserved | 1 | 1 | 2 | 2 | 97 | 93 | 190 | 98 |
| t-test | 0 | 0 | 2 | 0 | 74 | 79 | 77 | 76 |
| Outlier test | 0 | 0 | 0 | 0 | 0 | 0 | 4 | 0 |

B. Number of specific microbial probe signatures detected.

| | Total no. of | Total no. of | Detection in | |
|---|---|---|---|---|
| | probes in the | probes | triple negative | |
| Bacterial signatures | Chip | detected | breast tumors | |
| Members | Specific | Specific | Percent positive | Organism |
| Arcanobacterium | 4 | 4 | 75 | Bacteria |
| Brevundimonas sp. | 3 | 3 | 73.1 | Bacteria |
| Sphingobacteria | 5 | 5 | 67.3 | Bacteria |
| Providencia | 1 | 1 | 67.3 | Bacteria |
| Prevotella | 2 | 2 | 67.3 | Bacteria |
| Brucella | 10 | 10 | 65.4 | Bacteria |
| E. coli | 13 | 10 | 63.5 | Bacteria |
| Actinomyces | 4 | 4 | 51.9 | Bacteria |
| Mobiluncus | 4 | 4 | 50 | Bacteria |
| Propiniobacteria | 2 | 2 | 50 | Bacteria |
| Geobacillus | 2 | 1 | 44.2 | Bacteria |
| Rothia | 3 | 3 | 40.4 | Bacteria |
| Peptinophilus | 2 | 2 | 38.5 | Bacteria |
| Capnocytophaga | 1 | 1 | 36.55 | Bacteria |
| Pleistophora | 8 | 8 | 98.1 | Fungi |
| Piedra | 6 | 6 | 90.4 | Fungi |
| Foncecaea | 3 | 3 | 88.5 | Fungi |
| Phialophora | 4 | 4 | 86.5 | Fungi |
| Paecilomyces | 4 | 4 | 69.2 | Fungi |
| Trichuris sp. | 7 | 7 | 96.2 | Parasite |
| Toxocara sp. | 1 | 1 | 61.5 | Parasite |
| Leishmania sp. | 6 | 5 | 59.6 | Parasite |
| B. equi | 2 | 2 | 55.8 | Parasite |
| Thelazia sp. | 1 | 1 | 40.4 | Parasite |
| Paragonimus sp. | 3 | 2 | 15.4 | Parasite |

TABLE 4
Hybridization signal (calculated as sum of hybridization signal of all the probes per accession) and
prevalence of viral and microbial probes detected in 100 triple negative breast cancer samples.
The methods that detected the candidates are mentioned; AO: Accession outlier,
SO: Specific probe outliers, CO: Conserved probe outlier, CT: Conserved
probe t-test; MAT: Model based analysis for tiling arrays.

| | | | | |
|---|---|---|---|---|
| | Probe Sum/ | | Detection | Percent |
| Accessions | accession | Description | Methods | detected (%) |

(a.) Associated viral agent

| | | | | |
|---|---|---|---|---|
| NC_006273.2 | 14332000 | Human herpesvirus 5 | AO, SO, CO | 92.31 |
| NC_009333.1 | 12119800 | Human herpesvirus 8 | AO, SO | 96.15 |
| NC_001669.1 | 8113970 | Simian virus 40 | MAT, SO, AO | 75.00 |
| NC_004102.1 | 7199330 | Hepatitis C virus genotype 1 | MAT, SO | 90.38 |
| NC_001488.1 | 7040500 | Human T-lymphotropic virus 2 | MAT, SO, AO | 88.46 |
| NC_005336.1 | 6422460 | Orf virus | MAT, CO | 75.00 |
| NC_013804.1 | 5037880 | Pseudocowpox virus | MAT, CO, AO | 90.38 |
| NC_009334.1 | 5024970 | Human herpesvirus 4 | MAT, SO, AO | 78.85 |
| NC_005337.1 | 4214040 | Bovine papular stomatitis virus | MAT, CO, AO | 84.62 |
| NC_009532.1 | 3435060 | Okra mosaic virus | MAT, SO, AO | 75.00 |
| NC_001352.1 | 3361460 | Human papillomavirus - 2 | MAT, SO | 84.62 |
| NC_001436.1 | 2745990 | Human T-lymphotropic virus 1 | MAT, SO, AO | 82.69 |
| NC_003977.1 | 2621640 | Hepatitis B virus | MAT, SO, AO | 86.54 |
| NC_001806.1 | 2319570 | Human herpesvirus 1 | MAT, SO, AO | 65.38 |
| NC_001526.2 | 1651350 | Human papillomavirus type 16 | MAT, SO | 78.85 |
| NC_001501.1 | 1587600 | Moloney murine leukemia virus | MAT | 57.69 |
| NC_010277.1 | 1551830 | Merkel cell polyomavirus | MAT, SO, AO | 90.38 |
| NC_001503.1 | 1464980 | Mouse mammary tumor virus | MAT, SO, AO | 78.85 |
| NC_001355.1 | 1271950 | Human papillomavirus type 6b | MAT, SO, AO | 78.85 |
| NC_001357.1 | 1184610 | Human papillomavirus - 18 | MAT, SO | 75.00 |
| NC_001699.1 | 755288 | JC polyomavirus | MAT, AO | 76.92 |
| NC_001837.1 | 749098 | Hepatitis GB virus A | MAT, SO | 82.69 |
| NC_001403.1 | 691071 | Fujinami sarcoma virus | MAT, SO | 90.38 |

(b.) Associated bacterial agent

| | | | | |
|---|---|---|---|---|
| FM180525.1 | 2576810 | Brevundimonas diminuta | SO, AO | 73.08 |
| AJ576081.1 | 2574020 | Mobiluncus mulieris | SO, MAT | 50.00 |
| AJ576084.1 | 2403760 | Mobiluncus curtisii | SO | 50.00 |
| EF025325.1 | 1953410 | Geobacillus stearothermophilus | SO, AO | 44.23 |
| AJ937773.1 | 1862550 | Propionibacterium jensenii | SO, MAT | 50.00 |
| X82451.1 | 1673860 | A. meyeri | SO, AO | 51.92 |
| X73952.1 | 1662360 | A. haemolyticum | SO | 75.00 |
| D14147.1 | 610579 | Peptoniphilus indolicus | SO, AO | 38.46 |
| EU373423.1 | 605635 | Sphingobacterium siyangensis | SO | 67.31 |
| AY689230.1 | 3006760 | Prevotella nigrescens | MAT | 67.31 |
| EU660316.1 | 527788 | Providencia rettgeri | AO, AT | 67.31 |
| NC_010498.1 | 63,373 | E. coli | SO | 63.46 |
| AJ717364.1 | 2331350 | Rothia sp. | SO | 40.38 |
| NC_010167.1 | 533342 | Brucella sp. | SO | 65.38 |
| EU796886.1 | 511261 | Capnocytophaga sp. | SO, AO | 36.54 |

(c.) Associated fungal agent

| | | | | |
|---|---|---|---|---|
| AY016366.1 | 10572800 | Piedraia hortae | SO | 90.38 |
| AF050282.1 | 9822510 | Phialophora verrucosa | SO, MAT, AO, AT | 86.54 |
| AF050276.1 | 8826430 | Fonsecaea pedrosoi | SO, MAT, AO, AT | 88.46 |
| EF119339.1 | 3574040 | Pleistophora mulleri | MAT | 98.08 |
| DQ069288.1 | 473,385 | Paecilomyces reniformis | SO, MAT | 38.46 |

(d.) Associated parasitic agent

| | | | | |
|---|---|---|---|---|
| DQ118536.1 | 13100000 | Trichuris trichiura | AO | 96.15 |
| U94382.1 | 842305 | Toxocara canis | AT, SO | 61.54 |
| AF337897.1 | 496007 | Thelazia gulosa | SO | 40.38 |
| Z15105.1 | 480369 | B. equi | SO | 55.77 |
| XM_001686577.1 | 402042 | Leishmania major | SO | 59.62 |

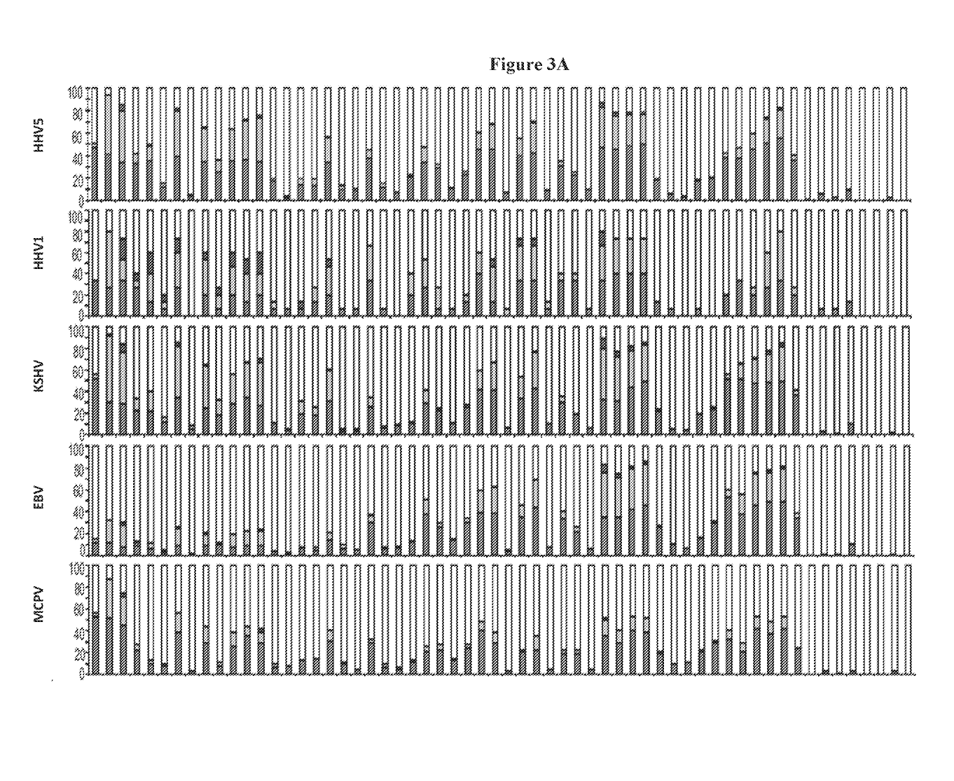

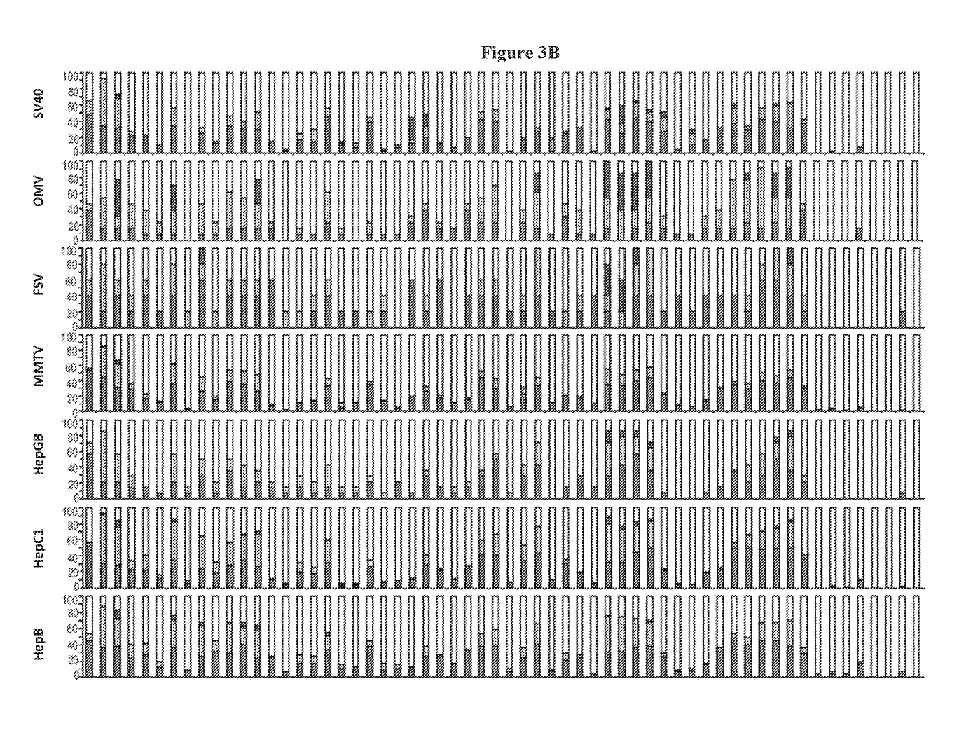

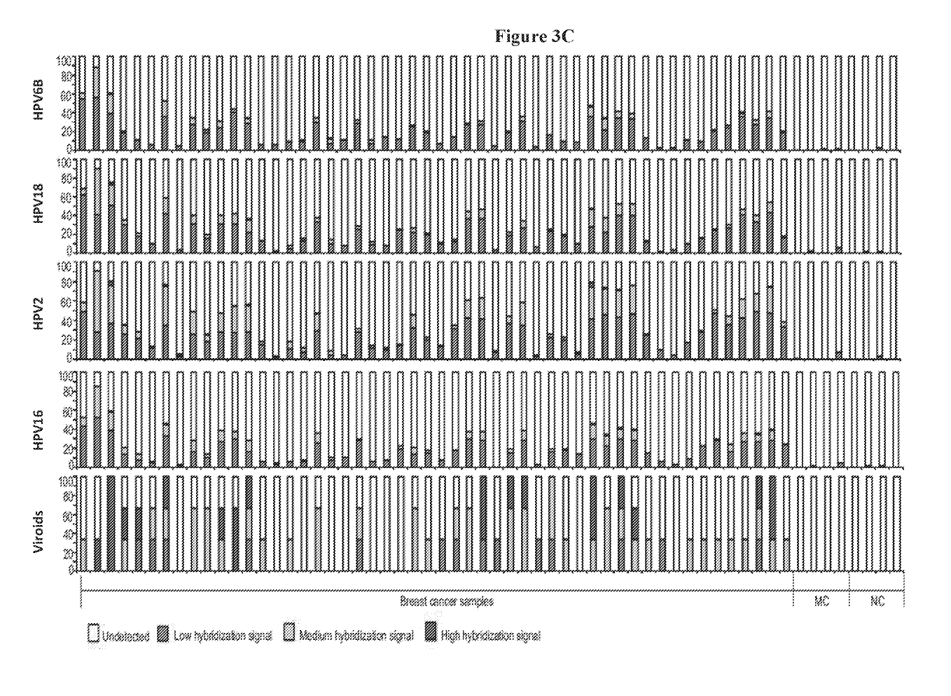

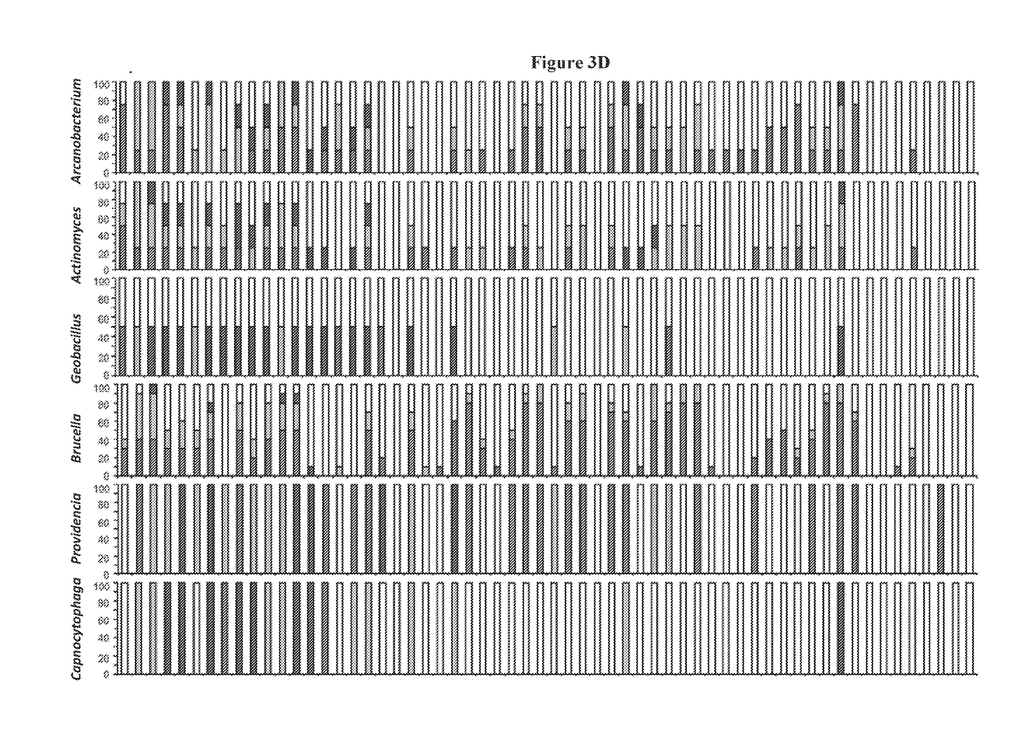

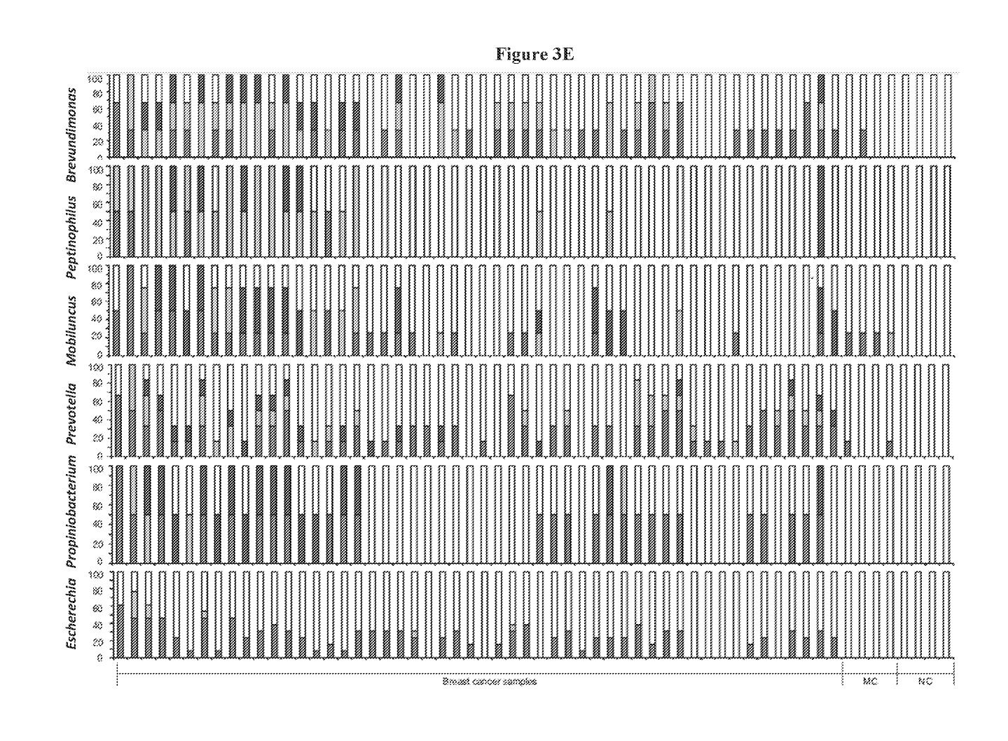

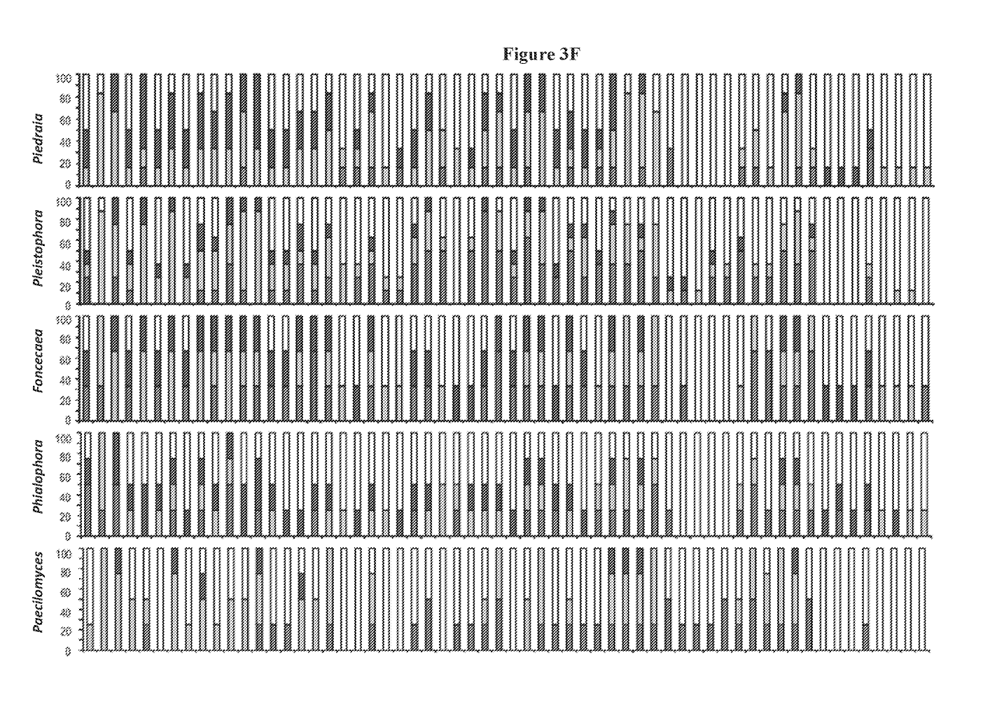

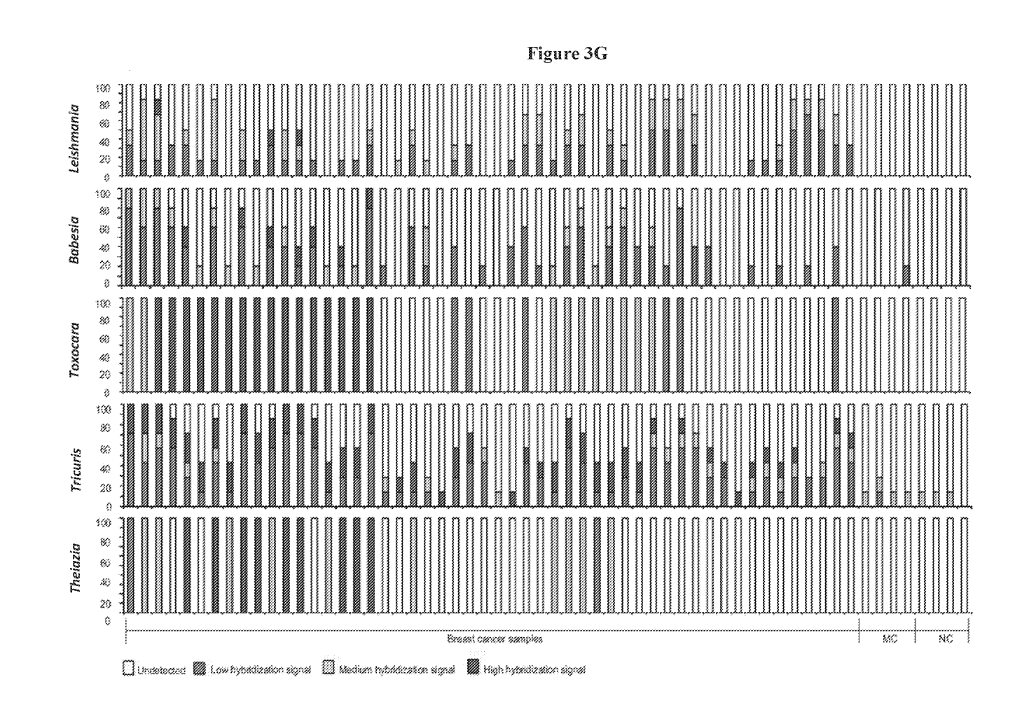

A probe was considered positive when the PathoChip screen showed a significantly higher hybridization signal for this probe in the cancer samples compared to matched or non-matched control samples (FIGS. 3A-3G; Table 5).

TABLE 5
Percent probes of microorganisms detected in
breast cancer samples versus the controls.

| | | p- value of | p- value of |
|---|---|---|---|
| | | detection in | detection in |
| | | breast cancer | breast cancer |
| | | vs. non-matched | vs. matched |
| | Microorganisms | controls | controls |
| Viruses | MMTV | 0.00049 | 0.00081 |
| | Hepatitis C1 | 0.00049 | 0.00154 |
| | EBV1 | 0.00049 | 0.00231 |
| | BPSV cp | 0.00049 | 0.00107 |
| | HCMV | 0.00050 | 0.00162 |
| | KSHV | 0.00050 | 0.00091 |
| | PCPV | 0.00052 | 0.00281 |
| | HPV2 | 0.00052 | 0.00091 |
| | HTLV-2 | 0.00052 | 0.00073 |
| | HPV6B | 0.00055 | 0.00049 |
| | MCPV | 0.00055 | 0.00061 |
| | HTLV1 | 0.00055 | 0.00309 |
| | HPV18 | 0.00055 | 0.00101 |
| | Hepatitis B | 0.00058 | 0.00294 |
| | SV40 | 0.00061 | 0.00138 |
| | HPV16 | 0.00069 | 0.00100 |
| | HHV1 | 0.00112 | 0.01300 |
| | Okra Mosaic Virus | 0.00114 | 0.00331 |
| | FSV | 0.00137 | 0.00061 |
| | Hepatitis GB | 0.00146 | 0.00090 |
| | MMLV | 0.00250 | 0.00250 |
| | Viroids | 0.00298 | 0.00298 |
| | Orf Virus | 0.00333 | 0.00333 |
| Bacteria | Prevotella | 0.00099 | 0.00412 |
| | Brevundimonas | 0.00155 | 0.00470 |
| | Arcanobacterium | 0.00181 | 0.00422 |
| | E. Coli | 0.00234 | 0.00234 |
| | Sphingobacterium | 0.00240 | 0.00240 |
| | Actinomyces | 0.00376 | 0.01282 |
| | Rothia | 0.00784 | 0.11245 |
| | Mobiluncus | 0.01263 | 0.30567 |
| | Propiniobacter | 0.01301 | 0.01301 |
| | Geobacillus | 0.03372 | 0.03372 |
| | Providencia | 0.04773 | 0.00419 |
| | Peptinophillus | 0.06186 | 0.06186 |
| | Capnocytophaga | 0.07289 | 0.07289 |
| Fungi | Pleistophora | 0.00069 | 0.00154 |
| | Paecilomyces | 0.00173 | 0.00490 |
| | Piedra | 0.00348 | 0.01329 |
| | Foncecaea | 0.01324 | 0.03374 |
| | Phialophora | 0.01470 | 0.13135 |
| Parasites | Trichuris | 0.00083 | 0.00159 |
| | B. equi | 0.00333 | 0.01007 |
| | Leishmania | 0.00410 | 0.00410 |
| | Toxocara | 0.00922 | 0.00922 |
| | Thelazia | 0.05771 | 0.05771 |

Table 5 shows the statistical significance of percent probes of candidate organisms detected in triple negative breast cancer samples vs. the matched and non-matched control samples. The significance is determined by Wilcoxon tests, and the percent detection of the pathogenic signatures in the cancer tissues were considered significant compared to the control tissues if the p value<0.05.

The viral, bacterial, fungal and parasitic signatures detected in the triple negative breast cancer samples were found to be significantly associated with the cancer samples (p<0.05) compared to the non-matched and matched control samples analyzed. The p-values for the association of the candidate organisms as determined by the probe signals in the cancer vs. the control tissues are provided in Table 5. Two different kinds of probe sets for viruses are contained in the PathoChip. The first are specific probes which are designed to detect a specific virus, for example probes that would detect human cytomegalovirus over all other herpesviruses. The second set are conserved probes which represent sequences that are highly conserved between members of a family of viruses or microorganisms, for example sequences conserved between all herpesviruses. The purpose for the conserved probes is to be able to detect heretofore unknown members of a family, for example a new human herpesvirus.

The probes of a candidate organism detected by the TNBC samples showed a wide range of hybridization signals across tumor samples (FIGS. 3A-3G). Here, the percentage of samples that had detectable hybridization signal (g−r>30) for each probe of an organism without differentiation of high or low signal was reported. Additionally, the names of specific viruses and microorganisms that were detected by specific probes on the PathoChip are listed. However, without being bound to a particular theory, detection by specific detection may suggest a closely related family member and not the specific one named. This is particularly relevant in cases where TNBC samples showed a range of hybridization signals across the probe set for a specific virus or microorganism. This could also mean that genomic regions of these aents are deleted in that particular tumor or a variance in a strain.

Among the conserved probes, viral signatures belonging to Herpesviridae, Retroviridae, Parapoxviridae, Polyomaviridae, Papillomaviridae families were detected. For the herpesviridae family, probes of Human Cytomegalovirus (HCMV), Human Herpesvirus 1 (HHV1; Herpes simplextype 1), Kaposi sarcoma herpes virus (KSHV), Epstein-Barr virus or Human Herpesvirus 4 (EBV/HHV4) were significantly detected among 92%, 65%, 96% and 78% of the breast cancer samples, respectively (FIGS. 4A-4B and Table 5). In the Poxviridae family, conserved probes for the parapoxviruses were significantly detected (p<0.05) in 83% of the triple negative breast cancer samples (FIGS. 4A-4B and Table 5). Among the retroviruses, specific probes of Fujinami Sarcoma virus (FSV) and Mouse mammary tumor virus (MMTV) were detected in 90.4% and 78.8% of the breast cancer samples, respectively (FIGS. 4A-4B and Table 5). Among the Polyomaviruses, specific probes detected signatures for Merkel cell Polyomavirus (MCPV) and SV40 in 90.3% and 75% of the breast cancer samples, respectively (FIGS. 4A-4B). For the papillomavirus family, specific probes detected HPV 6b, HPV18, HPV2 and HPV16 in 78.8%, 75%, 84.6%, and 78.8% of the breast cancer samples, respectively (FIGS. 4A-4B). Specific probes also detected signals for Hepatitis GB, C and B in 82.7%, 90.4%, and 86.5% of the cancer samples, respectively (FIGS. 4A-4B).

The viral probes detected, when ranked according to percent prevalence (regardless of hybridization intensity) showed signatures of Hapadnaviruses and Flaviviruses (86.5%), followed by Parapoxviruses (83.3%), Herpesviruses (83.2%), Retroviruses (79.6%),and Papillomaviruses (79.3%). However, when ranked according to decreasing hybridization signal (the total hybridization signal of individual probes per organism, i.e., Probe Sum/Accession), Herpesvirus probes had the highest hybridization signal across the tumors, followed by parapoxviruses, flaviviruses, polyomaviruses, retroviruses, hapadnaviruses and papilloma. (FIGS. 4A-4B and Table 4).

The bacterial signatures were detected in triple negative breast cancer samples and were ranked according to percent prevalence (FIGS. 4C-4D). For the bacterial signatures detected (FIGS. 4C-4D and Tables 3-4), the highest prevalence was of probes to detect Arcanobacterium (75%), followed by probes detecting the 16S rRNA signatures of Brevundimonas, Sphingobacteria, Providencia, Prevotella, Brucella, Escherichia, Actinomyces, Mobiluncus, Propiniobacteria, Geobacillus, Rothia, Peptinophilus, and Capnocytophaga (FIGS. 4C-4D). The bacterial probes of Prevotella showed the highest hybridization signal, followed by very high hybridization signals for probes of Brevundimonas, Mobiluncus, Rothia, Geobacillus, Propiniobacteria, Actinomyces and Arcanobacterium; moderate hybridization signal for probes of Peptinophilus, Sphingobacteria, Brucella, Providencia and Capnocytophaga and low hybridization signal for probes of Escherichia.

The fungal signatures were of rRNA probes that recognize Pleistophora which were detected in 98% of the breast cancer samples, followed by probes of Piedra, Foncecaea, Phialophora and Paecilomyces (FIGS. 4C-4D and Table 4). The highest hybridization signal was seen for the probes of Piedra, followed by high hybridization signal in probes for Phialophora, Foncecaea and Pleistophora and moderate hybridization signal for probes of Paecilomyces (FIGS. 4C-4D).

Probes detecting the parasitic signatures of Trichuris were detected in 96% of the triple negative breast cancer samples, followed by Toxocara, Leishmania, Babesia and Thelazia (FIGS. 4C-4D and Table 4). Based on the ranking of hybridization signal, probes of Trichuris showed the highest hybridization signal, followed by high hybridization signal for probes of Toxocara and moderate hybridization signal for Thelazia, Babesia and Leishmania.

### Hierarchical Clustering Reveals Two Distinct Microbial Signatures in TNBC Samples

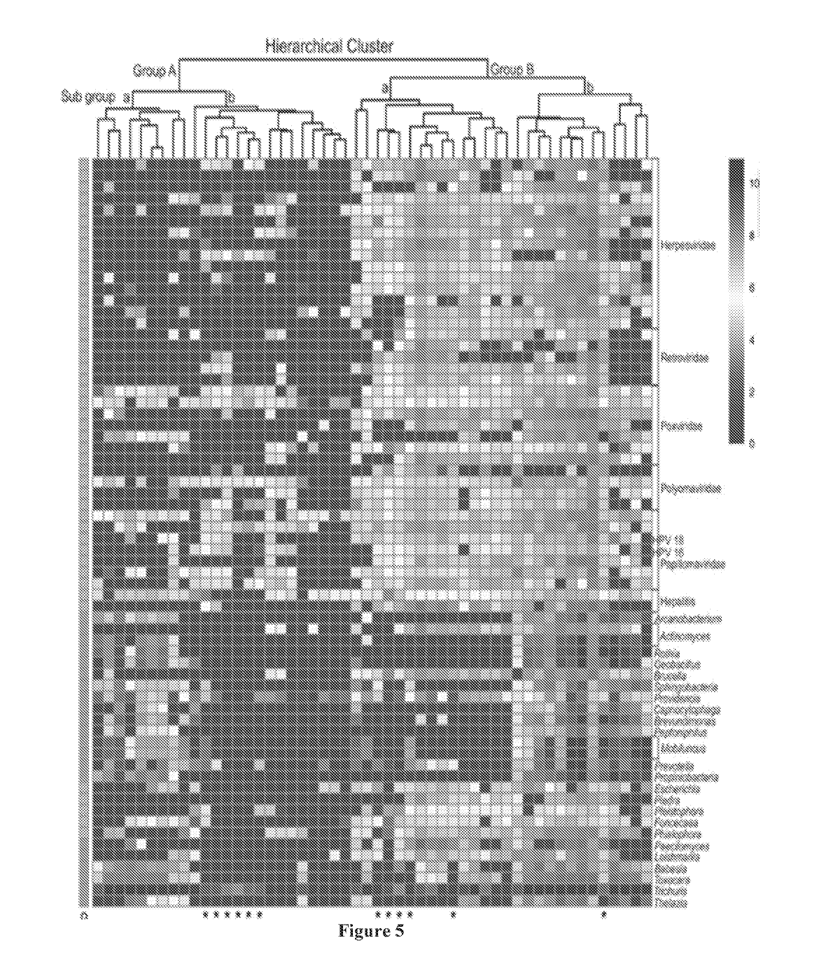

To determine if there were similarities in detection within tumor samples hierarchical clustering of the results of screening the 100 breast cancer samples (52 arrays) were performed. This analysis clustered the samples into two broad groups (FIG. 5). Group B showed strong hybridization signals for probes detecting viruses and fungi compared to group A TNBC samples. The group B TNBC samples were further categorized based on signals for bacteria and parasitic agents, which was found to be low in subgroup a and higher in subgroup b. Within the group A TNBC samples, some samples (subgroup a) had higher detection of probes for bacteria and parasites than others (subgroup b). Notably, probes for the parasite Trichuris was detected in almost all the TNBC samples screened. However, the phenotypic reason for the two distinct signatures was not immediately clear since the TNBC samples tested were de-identified.

### PCR Validation of Signatures Detected by PathoChip

PCR primers for several viruses, as well as a prevalent bacteria (Brevundimonas), fungus (Pleistophora) and parasite (Trichuris), were designed based on sequences from the conserved and specific PathoChip probes which showed moderate to high hybridization signals in the PathoChip screen for these viruses and organisms. As an example of these data, the papillomavirus conserved primers 7 and 8 which were designed from the conserved probes of papillomaviruses showed significant hybridization for many of the samples. The PCR results show the expected amplicons for samples Br15, Br16 and Br38 which were positive for those papillomavirus probes in the PathoChip screen. Conversely, sample Br18 was negative for these probes in the PathoChip screen and was also negative by PCR (FIGS. 6A-6C). In all the cases tested (FIGS. 6A-6C), the PCR amplification showed the expected amplicons for the PathoChip-detected viruses, as well as the selected bacterium, fungus and parasite (FIGS. 6A-6C). Sequencing of the PCR products verified the detection of the appropriate virus or other microorganism. Likewise, the samples that were negative by PathoChip screens for a particular virus or organism were negative in the PCR analysis. These data validate the results from the PathoChip screen supporting the presence of these microorganisms in TNBC samples.

Probe Capture for Target Sequencing to Identify the Signature Organisms Associated with Triple Negative Breast Cancer.

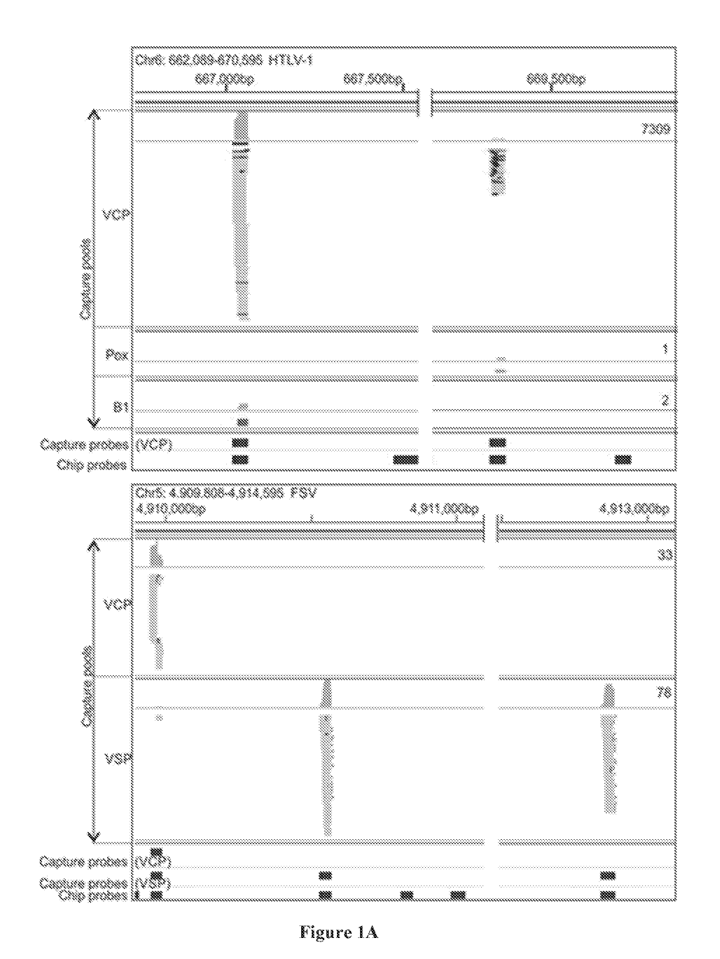

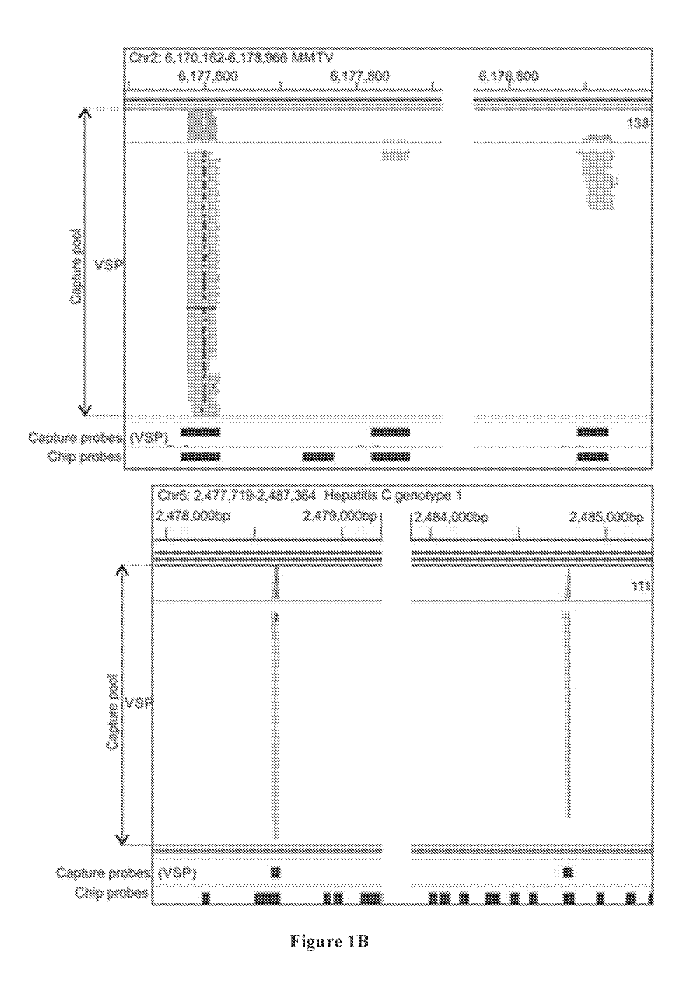

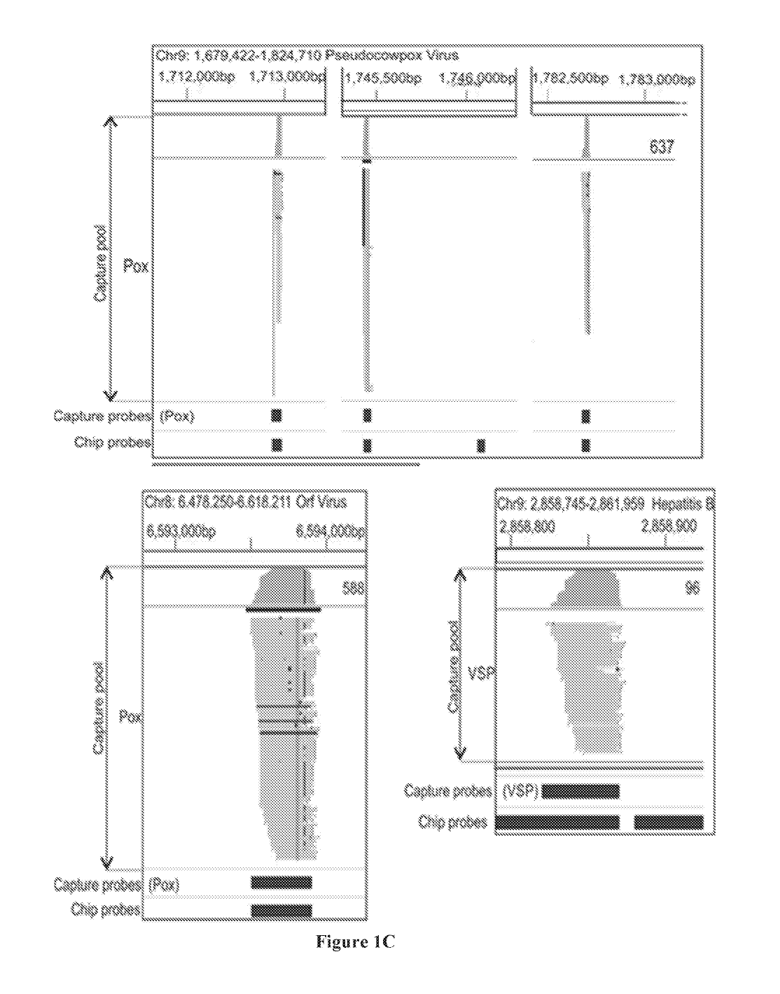

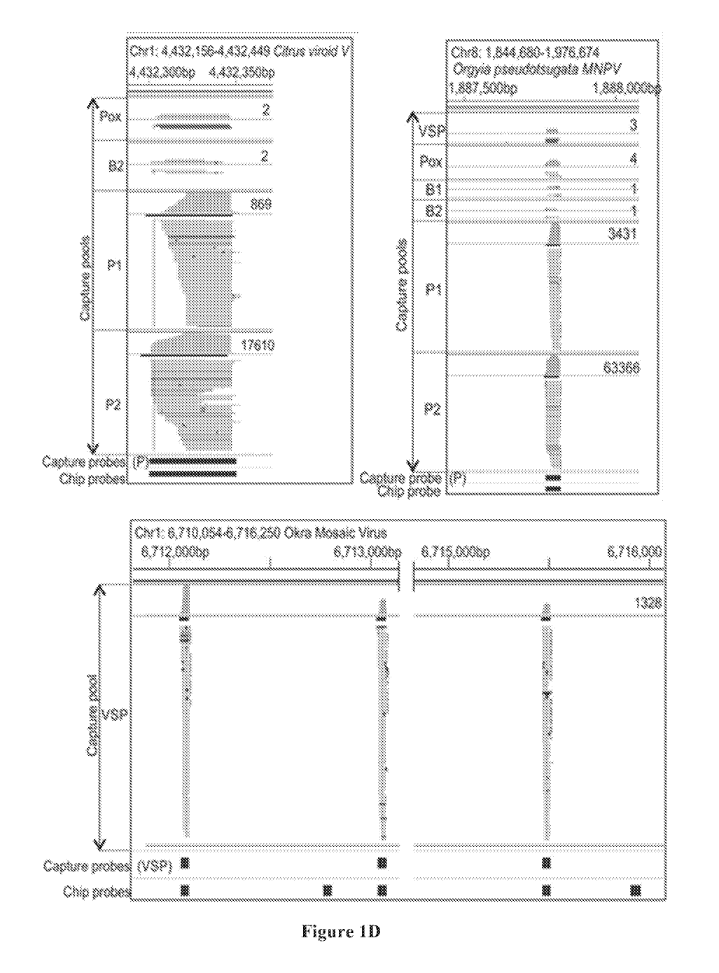

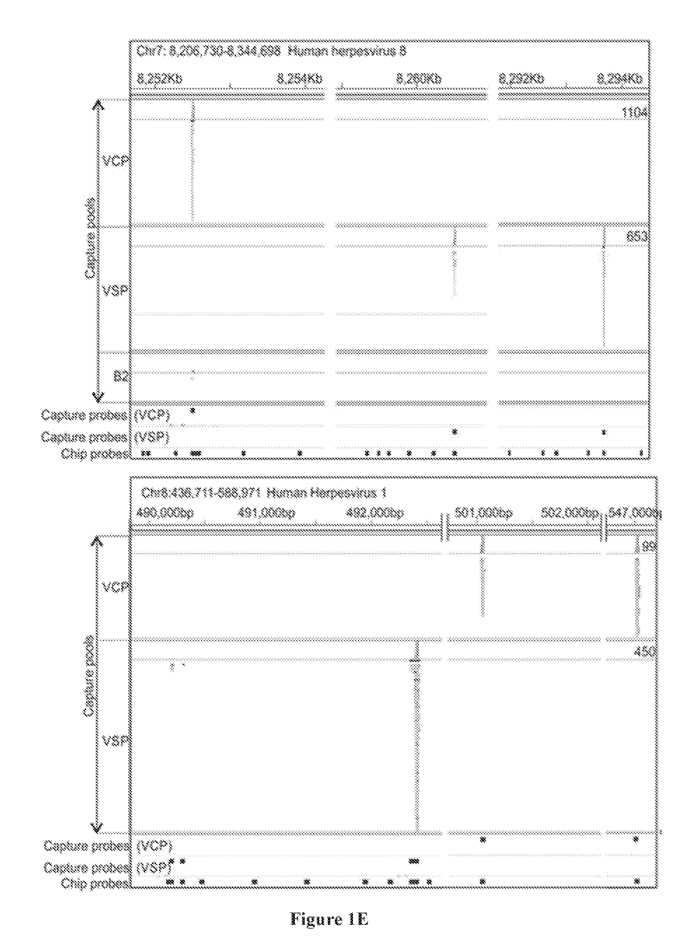

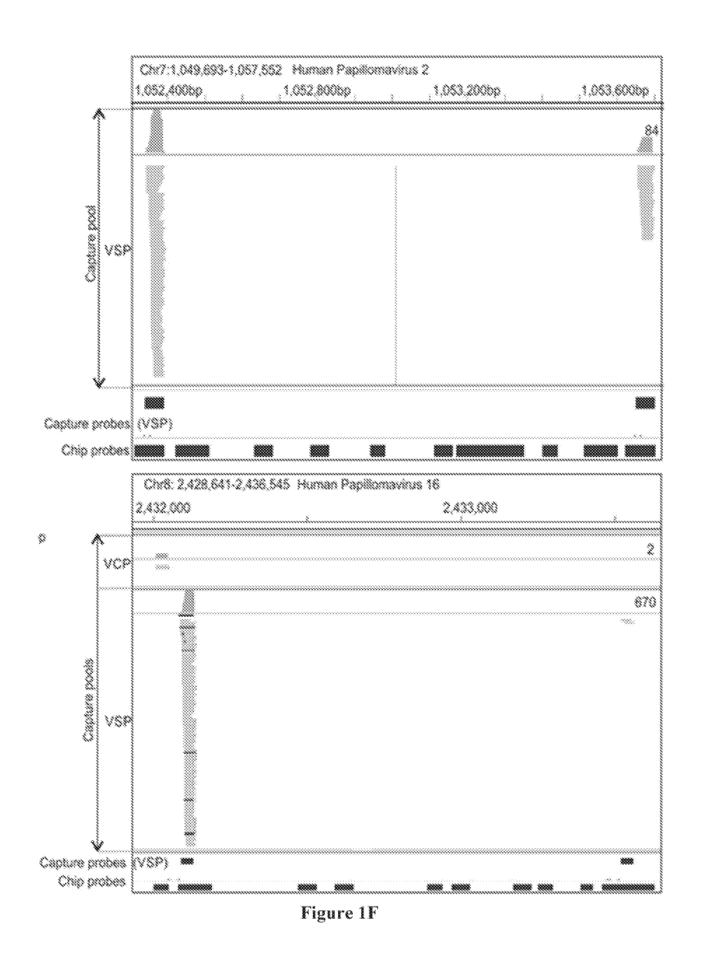

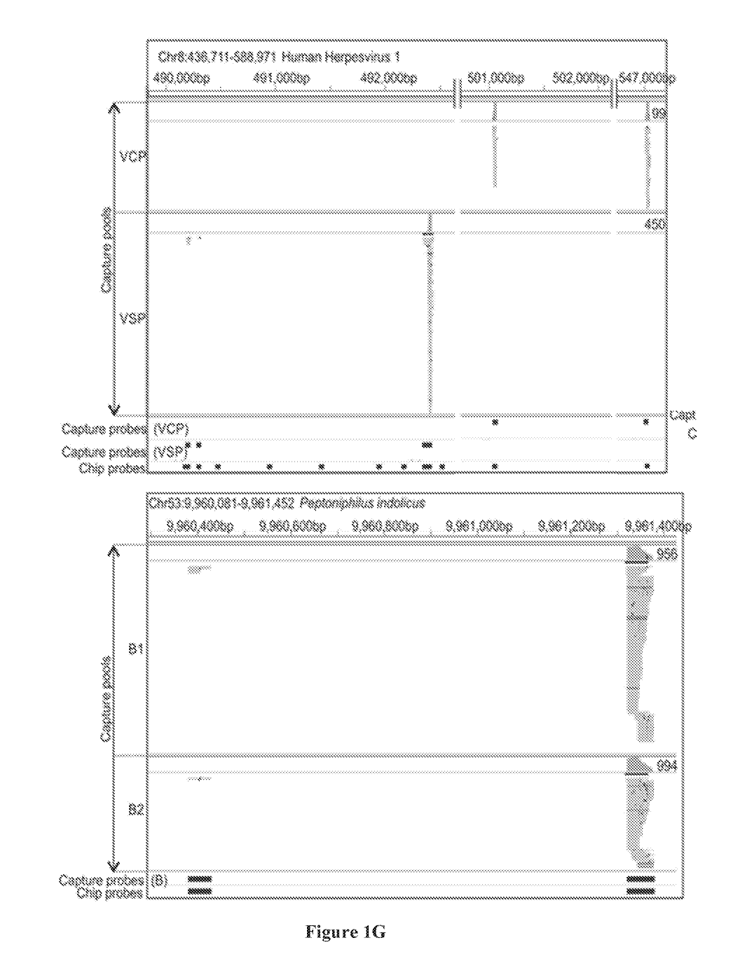

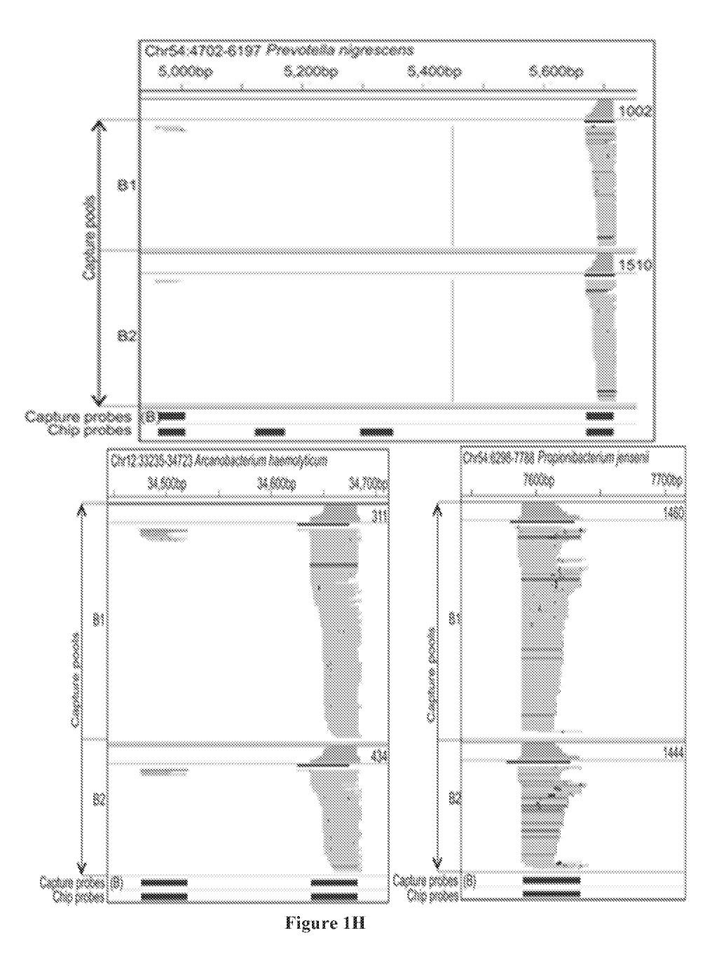

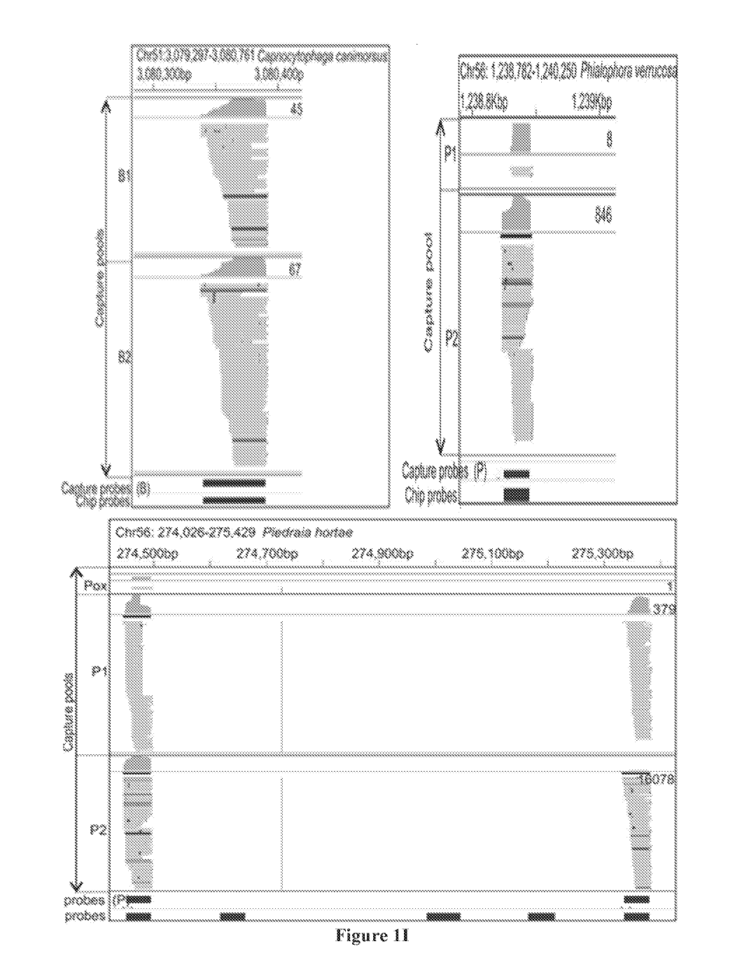

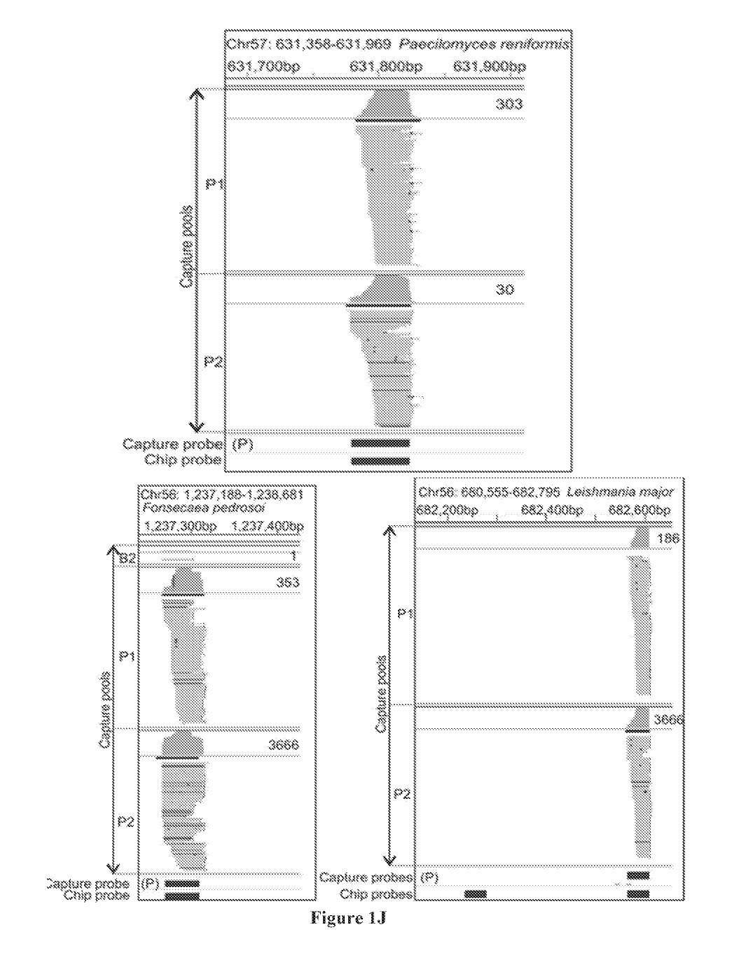

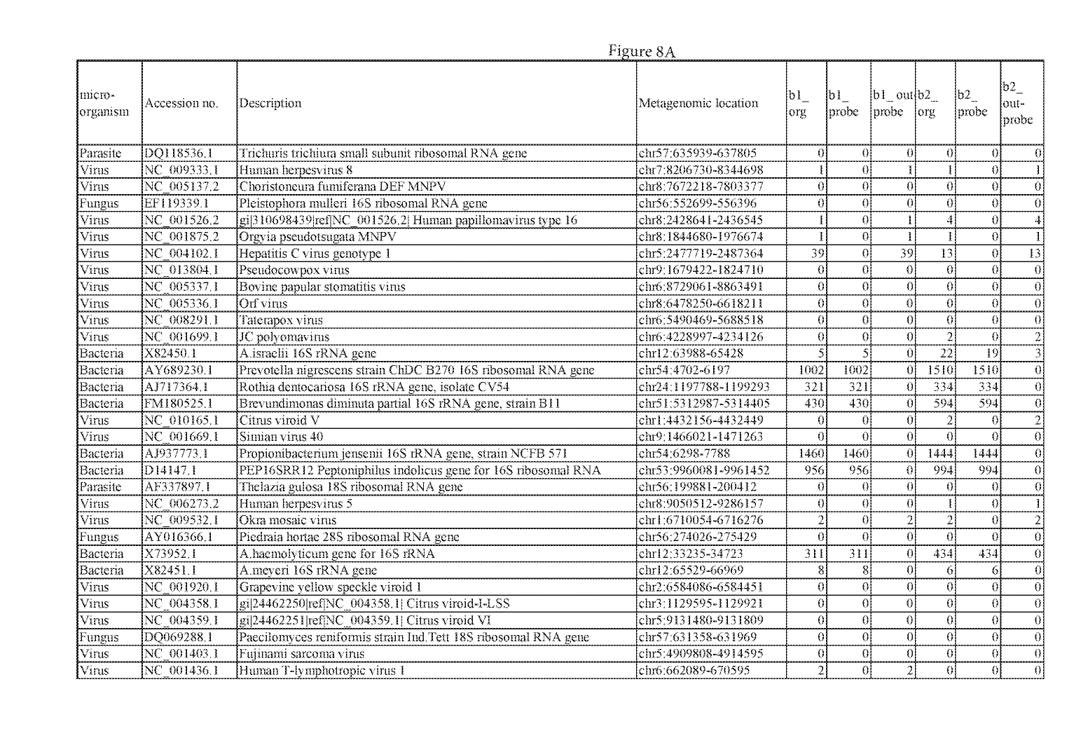

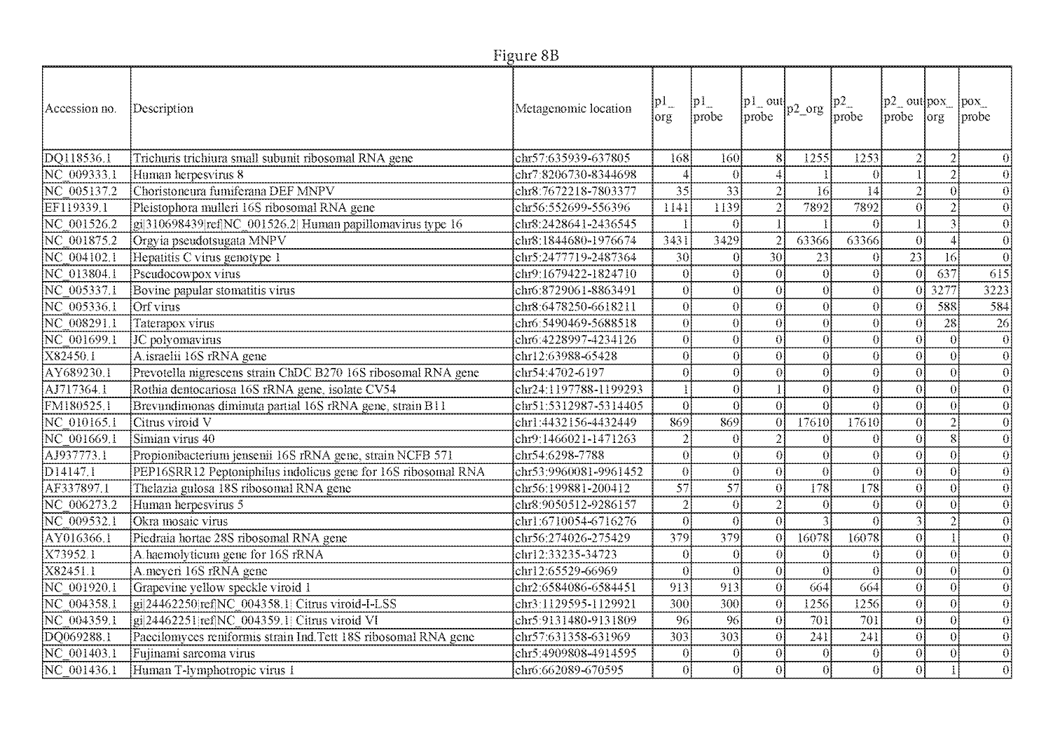

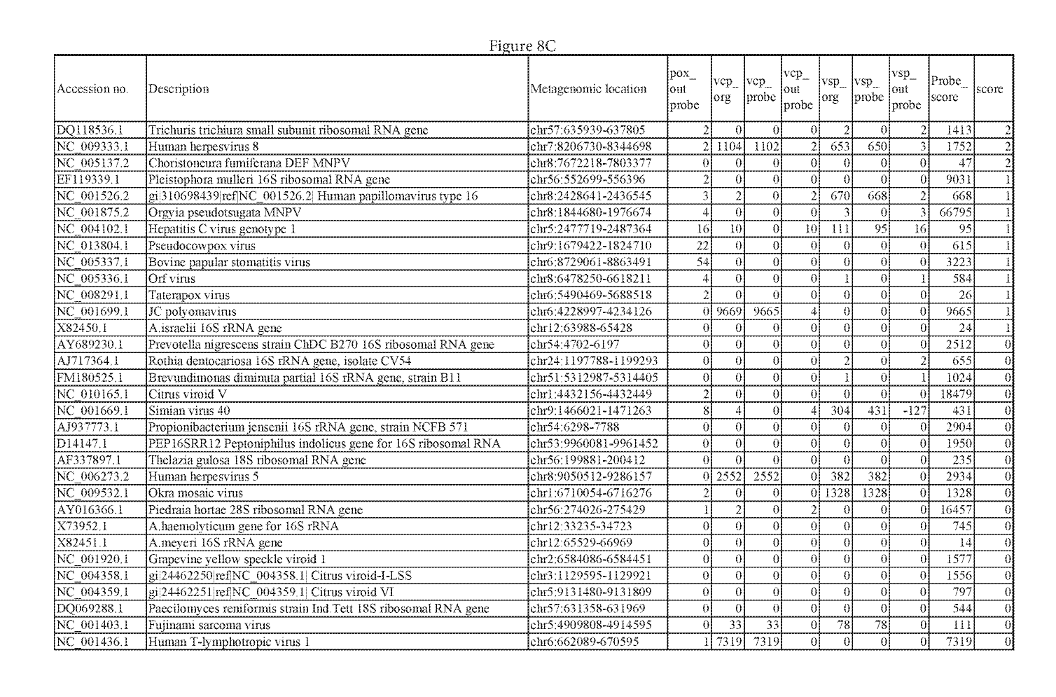

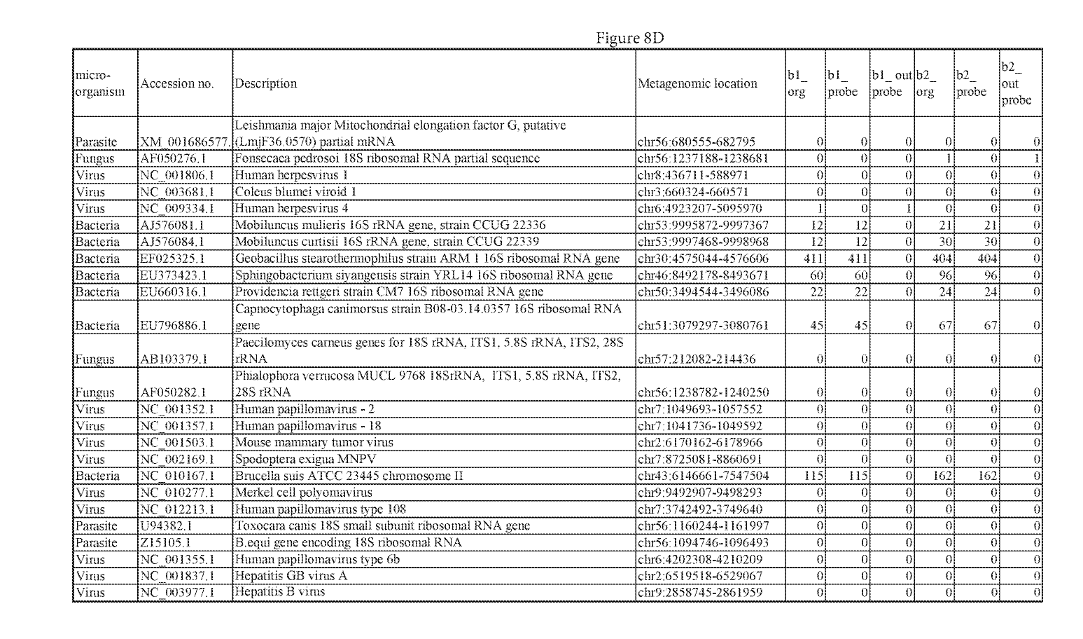

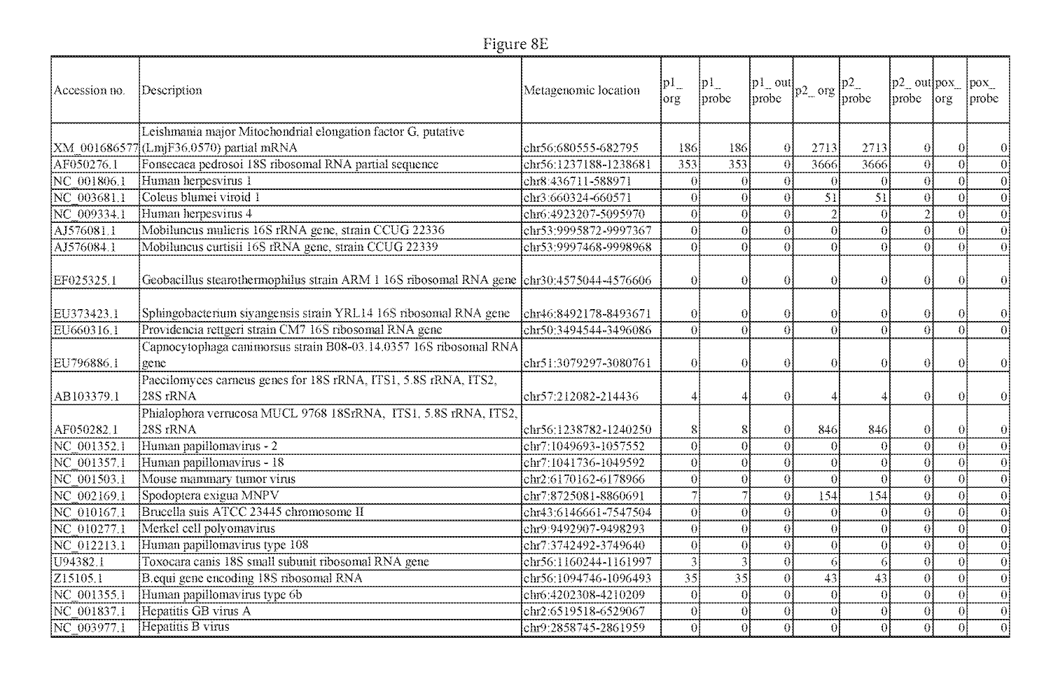

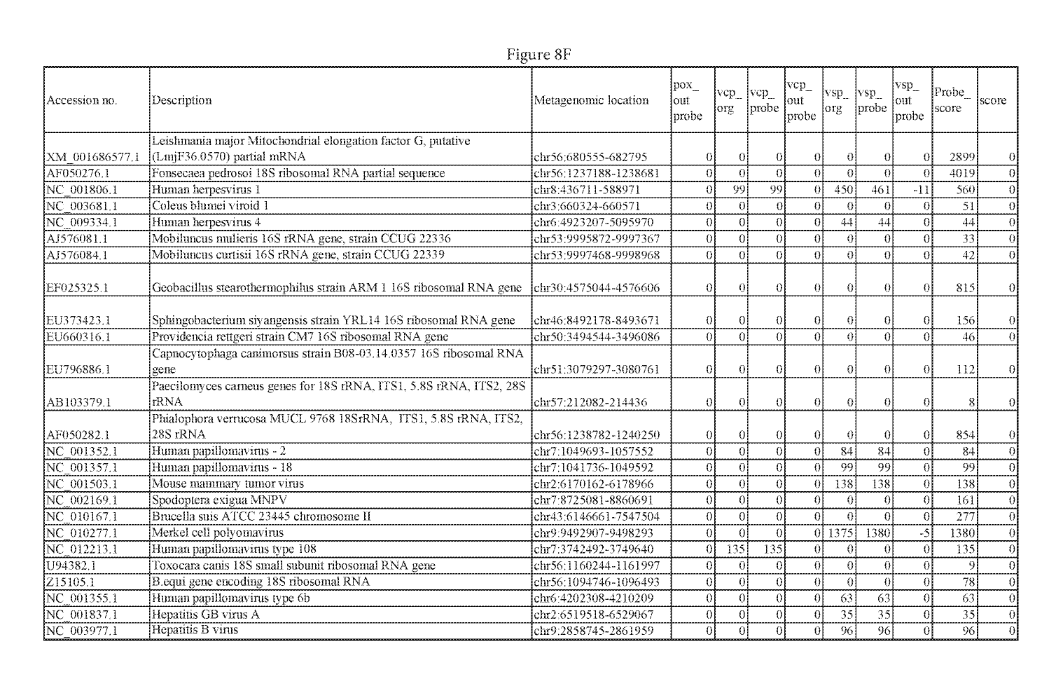

For additional validation of the PathoChip detection of viruses, bacteria, fungi and parasites in the TNBC samples, probes with stronger hybridization signal with the breast cancer samples and not in the controls were selected for target capture and sequencing. Hybridization signals for those probes across all the triple negative breast cancer, matched and non-matched controls analyzed in the study are presented as a heat map in FIG. 7A. Five probe pools (probe pool 1-5) were used to capture the targets from the pooled samples. Seven target capture reactions were performed with the 5 probe pools (FIGS. 7A-7D) [Viral Conserved Probe (VCP) capture, Pox capture, Viral Specific Probe (VSP) Bacterial probe captures (B1 and B2) and Fungal/Parasitic/Viroid probe captures (P1 and P2)]. The seven captured targets sequencing libraries were made, pooled and sequenced using MiSeq. The MiSeq data were aligned with the PathoChip metagenome. The data showed that the Miseq reads clustered, in large part, around the genomic locations of the probes used in the capture reactions; although occasionally regions of the target genomes outside the locations of the probe were detected (FIGS. 7B-7D). The number of MiSeq reads of the candidate organisms for each capture is shown in FIGS. 1A-1J and 8A-8F.

### Viral Genomes.

The MiSeq reads confirmed the presence of viral genomic regions of polyoma viruses (SV40, JC, MCPV); herpesviruses (HCMV); papilloma viruses (HPV16, HPV18, HPV2); retroviruses (HTLV1, MMTV), Pox Viruses (Pseudo cowpox virus, Bovine papular stomatitis virus and Orf virus) (FIGS. 1A-1J).

One of the most prevalent MiSeq reads (9669) aligned to a non-coding regulatory region of JC polyomavirus and was selected by a virus conserved probe (VCP) capture. In addition, target capture using specific probes of SV40 and MCPV revealed 304 and 1375 Miseq reads that mapped to the large T-antigen genes of SV40 and MCVP, respectively. These data support the association of a polyoma-like virus with triple negative breast cancer. VCP capture also resulted in 2,552 MiSeq reads which mapped to UL70 (primase) and UL104 (capsid) of HCMV and specific probe capture yielded 382 reads that mapped to the HCMV non-coding RNA 4.9, as well as the UL77 and UL98 genes. Specific probes capture resulted in 670 reads which aligned to the E2, E4 and L2 region of HPV16 genome and 99 reads that aligned to the L1 region of HPV18 genome. Additionally, HPV-2 sequences were indicated by 86 reads aligned to HPV-2 E1 as well as the genomic sequences between the HPV-2 E4 and L2 genes. Hepatitis viral genomes were indicated by 111 reads that aligned with the probe sequence within the E1/E2 polyprotein and the non-structural 5A genomic sequence of the Hepatitis C genotype 1. Ninety-six (96) reads aligned with the probe corresponding to the S protein of Hepatitis B. Retroviral genomes were detected by VCP capture where 7,319 reads aligned to the Rex/Tax and env genes of HTLV-1; and 33 and 78 reads from the VCP and specific viral probe capture mapped to the p140 polyprotein gene of Fujinami sarcoma virus (Petropoulos (1997) Retroviral Taxonomy, Protein Structures, Sequences, and Genetic Maps. In: Coffin J M, Hughes S H, Varmus H E, editors. Retroviruses. Cold Spring Harbor (N.Y.): Cold Spring Harbor Laboratory Press). Further, specific probe capture yielded 138 sequence reads that aligned to the super-antigen and pol/env genes of mouse mammary tumor virus (Petropoulos (1997) Retroviral Taxonomy, Protein Structures, Sequences, and Genetic Maps. In: Coffin J M, Hughes S H, Varmus H E, editors. Retroviruses. Cold Spring Harbor (N.Y.): Cold Spring Harbor Laboratory Press). Poxviral genomic regions were indicated by VCP capture where 637 reads aligned to the DNA polymerase and tyrosine phosphatase genes of pseudocowpox virus, 3,277 reads aligned to the ORF041 (hypothetical protein), the ORF044 (core protein) and ORF064 (mRNA capping enzyme large sub-unit) of the Bovine Papular Stomatitis Virus, and 588 reads aligned to the to the hypothetical protein encoding gene of Orf virus.

### Bacterial Genomes.

Specific bacterial probes used for target capture and sequencing resulted in MiSeq reads that aligned to the 16S rRNA genomic locations of the bacterial signatures that were detected by the PathoChip screen; namely, Brevundimonas diminuta, Arcanobacterium haemolyticum, Peptoniphilus indolicus, Prevotella nigrescens, Propiniobacterium jensenii and Capnocytophaga canimorsus (FIGS. 1A-1J, and FIGS. 8A-8F).

### Fungal and Parasite Genomes.

The fungal and parasitic pooled probes (P) captured targets that mapped to rRNA genes of the following fungal organisms: Pleistophora mulleris, Piedraia hortae, Paecilomyces reniformis, Phialophora verrucosa and Fonsecaea pedrosoi; and the 18S rRNA regions following parasites: Trichuris trichura, Thelazia gulosa and Leishmania major (FIGS. 1A-1J, 7B-7D, and 8A-8F).

The PathoChip screening data are in agreement with the findings of other reports that suggest the association of viruses with a variety of cancers. For example, previous studies suggest the presence of herpesvirus, papillomavirus, polyomavirus and MMTV-like sequences in breast cancer (Alibek et al. (2013) Infect Agent Cancer 8, 32; de Martel & Franceschi (2009) Crit Rev Oncol Hematol 70, 183-194; Porta et al. (2011) Cancer Lett 305, 250-262; Harkins et al. (2010) Herpesviridae 1, 8; Amarante and Watanabe (2009) J Cancer Res Clin Oncol 135, 329-337; Mazouni et al.(2011) Br J Cancer 104, 332-337; Piana et al. (2014) Virol J 11, 190; Pogo and Holland (1997) Biol Trace Elem Res 56, 131-142; Salmons et al. (2014) J Gen Virol 95, 2589-2593). One study reported a much higher rate of HCMV infection (97%) in biopsy specimens of breast cancer patients compared to controls by immunohistochemistry (Harkins L E, Matlaf L A, Soroceanu L, Klemm K, Britt W J, Wang W, Bland K I, & Cobbs C S (2010) Herpesviridae 1, 8). Others have reported EBV DNA from breast cancer samples by PCR and suggested the association of EBV with more severe forms of breast cancer (Alibek et al. (2013) Infect Agent Cancer 8, 32; Amarante and Watanabe (2009) J Cancer Res Clin Oncol 135, 329-337; Mazouni et al. (2011) Br J Cancer 104, 332-337). A study examining 1,535 cases, showed significant association of EBV with increased breast cancer risk (Huo et al. (2012) PLoS One 7, e31656). SV40 DNA sequence from the T antigen gene were reported in 22% of 109 breast cancer samples as determined by PCR with confirmation by immunohistochemistry (Alibek et al. (2013) Infect Agent Cancer 8, 32). Furthermore JCV, another polyomavirus, was detected in 23% of 123 breast cancer cases by PCR (Hachana et al. (2012) Breast Cancer Res Treat 133, 969-977). Additionally, the association of high risk HPV with breast cancer has been suggested (Simoes et al. (2012) Int J Gynecol Cancer 22, 343-347). A recent study detected HPV in 15% of triple negative breast cancer patients (40 cases) but not in 40 non-triple negative cases by PCR (Hachana et al. (2012) Breast Cancer Res Treat 133, 969-977). The most frequent genotype detected was HPV-16 (28.6%), and others were HPV-31, -45, 52, -6, -66 (Piana et al. (2014) Virol J 11, 190).

Other studies have proposed an association between the beta-retrovirus human mammary tumor virus (HMTV) and breast cancer. This is due to the detection of MMTV-like sequences in breast cancer samples and not in normal tissues (Pogo B G & Holland J F (1997) Biol Trace Elem Res 56, 131-142); HMTV has 95% sequence homology with MMTV (Bittner and Imagawa (1953) Cancer Res 13, 525-528). The env, gag and sag HMTV gene sequences from patients with breast cancer have been cloned and sequenced suggesting the existence of this virus in breast cancer patients (Zenit-Zhuravleva et al. (2012) European Journal of Cancer 48). That multiple viruses can co-exist in the same breast cancer sample has been suggested by studies showing the presence and co-existence of EBV (68%), HPV (50%) and MMTV (78%) (Alibek et al. (2013) Infect Agent Cancer 8, 32). In sum these data suggest a substantial presence of viruses in tumor tissue. The PathoChip screen of TNBC indicates that many of these viral signatures are associated with one specific cancer, TNBC, along with the presence of signatures for bacteria, parasites and fungi.

It is interesting that TNBC samples fell into hierarchical groups showing at least two distinct microbial signatures. One hierarchical group (group B) was prevalent in viruses: a herpesvirus-signature (primarily β- and γ-herpesvirus-like); a parapoxvirus signature (parapox virus family-like); flavivirus (hepatitis C- and GB-like); polyomavirus (JC-MCPV- and SV40-like); retrovirus (MMTV-, HERV-K-, HTLV-like); hepadnavirus (hepatitis B-like) and papillomavirus (HPV-2, 6b and 18-like). This hierarchical group also tended to be higher in fungal signatures and suggested representatives of the Pleistophora, Piedraia, Fonsecaea, Phialophora and Paecilomyces families. Bacterial and parasitic signatures could be found equally between the two hierarchical groups. Bacterial probes included representatives of a number of families (Actinomycetaceae, Caulobacteriaceae, Sphingobacteriaceae, Enterobacteriaceae, Prevotellaceae, Brucellaceae, Bacillaceae, Peptostreptococcaceae, Flavobacteriaceae), some of which have been associated with cancers and parasitic signatures included representatives of the Trichuris (highly detected in most of the TNBC samples screened), Toxocara, Leishmania, Thelazia and Babesia families. In fact, there has been one report on the association of parasites with metastatic breast cancer38. It is interesting that the associated viral signatures may provide clues as to a potential pathogenic role based on previous reports. The fact that there are two distinct groups based on the hierarchical analysis suggests a possible separation of TNBC based on associated microorganisms. Nevertheless, future studies characterizing these groups will be critical to provide further insights into the disease.

In sum, the targeted probe capture and sequencing data support the results of the PathoChip screen suggesting that genomic signatures for the detected viruses, other microorganisms, or their closely related family members, are much more frequently associated with TNBC tissues than normal tissues.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, with art-recognized alternatives and using no more than routine experimentation, are within the scope of the present application.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of detecting triple negative breast cancer in a tumor tissue sample from a subject, the method comprising:
hybridizing a detectably-labeled nucleic acid from the tumor tissue sample to a PathoChip array to generate a first hybridization pattern;
hybridizing a detectably-labeled nucleic acid from a reference sample to a PathoChip array to generate a second hybridization pattern, wherein the reference sample is from an otherwise identical non-tumor tissue from a subject;
comparing the first and second hybridization patterns, wherein when the first hybridization pattern is substantially a microbial hybridization signature and the second hybridization pattern is substantially not a microbial hybridization signature, triple negative breast cancer is detected in the tumor tissue sample.

2. The method of claim 1, wherein the microbial hybridization signature is generated by hybridization of the detectably-labeled nucleic acid from the tumor tissue sample to at least three nucleic acid probes on the PathoChip, wherein the probes are from microbes selected from the group consisting of Mouse mammary tumor virus (MMTV), Human T-Lymphotropic virus type I (HTLV-1), Fujinami Sarcoma virus (FSV), Simian virus 40 (SV40), John Cunningham virus (JC), Merkel cell Polyomavirus (MCPV), Human Cytomegalovirus (HCMV), Epstein-Barr virus (EBV), Kaposi's sarcoma-associated herpesvirus (KSHV), Human papillomavirus 16 (HPV16), Human papillomavirus 6b (HPV6b), Hepatitis B virus (HBV), Hepatitis C virus (HCV-1), Bovine papular stomatitis virus (BPSV), Pseudocowpox virus (PCP), Taterapox virus (Tatera), Orf virus (Orf), Arcanobacterium, Brevundimonas sp, Sphingobacteria, Providencia, Prevotella, Brucella, Escherichia coli (E. coli), Actinomyces, Mobiluncus, Propiniobacteria, Geobacillus, Rothia, Peptinophilus, Capnocytophaga, Pleistophora, Piedra, Foncecaea, Phialophora, Paecilomyces, Trichuris sp., Toxocara sp., Leishmania sp., Theileria equi (B.equi), Thelazia sp., or Paragonimus sp.

3. The method of claim 1, wherein the first hybridization pattern is generated by hybridization of the detectably-labeled nucleic acid from the tumor tissue sample to at least three nucleic acid probes on the PathoChip, wherein the probes are selected from the group consisting of SEQ ID NOS: 1-160.

4. A method of detecting triple negative breast cancer in a tumor tissue sample from a subject, the method comprising:
hybridizing a detectably-labeled nucleic acid from the tumor tissue sample to a first microarray comprising at least three nucleic acid probes selected from the group consisting of SEQ ID NOS: 1-160 to generate a first hybridization pattern;
hybridizing a detectably-labeled nucleic acid from a reference sample to a second microarray comprising at least three nucleic acid probes selected from the group consisting of SEQ ID NOS: 1-160 to generate a second hybridization pattern, wherein the reference sample is from an otherwise identical non-tumor tissue from a subject;
comparing the first and second hybridization patterns, wherein when the first hybridization pattern is substantially a microbial hybridization signature and the second hybridization pattern is substantially not a microbial hybridization signature, triple negative breast cancer is detected in the tumor tissue sample.

5. The method of claim 1, wherein the tumor tissue sample is selected from the group consisting of a biopsy, formalin-fixed, paraffin-embedded (FFPE) sample, or non-solid tumor.

6. The method of claim 1, wherein the subject is human.

7. The method of claim 1, wherein the detectably-labeled nucleic acid is labeled with a fluorophore, radioactive phosphate, biotin, or enzyme.

8. The method of claim 7 wherein the fluorophore is Cy3 or Cy5.

9. The method of claim 1, further comprising wherein when triple negative breast cancer is detected in the tumor tissue sample from a subject, the subject is provided with a treatment for triple negative breast cancer.

10. The method of claim 9, wherein the treatment comprises surgery, chemotherapy, or radiotherapy.

11. A composition comprising at least three nucleic acid probes selected from the group consisting of SEQ ID NOS: 1-160.

12. A microarray comprising at least three nucleic acid probes selected from the group consisting of SEQ ID NOS: 1-160.

13. The microarray of claim 12, wherein the nucleic acid probes are selected from about 10 to about 30 microbes and comprise about 3 to about 5 probes per microbe.

14. A microarray comprising at least three nucleic acid probes selected from the group of microbes consisting of MMTV, HTLV-1, FSV, SV40, JC, MCPV, HCMV, EBV, KSHV, HPV16, HPV6b, HBV, HCV-1, BPSV, PCP Tatera, Orf, Arcanobacterium, Brevundimonas sp, Sphingobacteria, Providencia, Prevotella, Brucella, E. coli, Actinomyces, Mobiluncus, Propiniobacteria, Geobacillus, Rothia, Peptinophilus, Capnocytophaga, Pleistophora, Piedra, Foncecaea, Phialophora, Paecilomyces, Trichuris sp., Toxocara sp., Leishmania sp., B.equi, Thelazia sp., Paragonimus sp.

15. The composition of claim 12, wherein the microarray is a biochip, glass slide, bead, or paper.

16. A kit comprising at least three nucleic acid probes selected from the group consisting of SEQ ID NOS: 1-160, and instructional material for use thereof.

17. A kit comprising a microarray comprising at least three nucleic acid probes selected from the group consisting of SEQ ID NOS: 1-160, and instructional material for use thereof.

18. A kit comprising a microarray comprising at least three nucleic acid probes selected from the group of microbes consisting of MMTV, HTLV-1, FSV, SV40, JC, MCPV, HCMV, EBV, KSHV, HPV16, HPV6b, HBV, HCV-1, BPSV, PCP Tatera, Orf, Arcanobacterium, Brevundimonas sp, Sphingobacteria, Providencia, Prevotella, Brucella, E. coli, Actinomyces, Mobiluncus, Propiniobacteria, Geobacillus, Rothia, Peptinophilus, Capnocytophaga, Pleistophora, Piedra, Foncecaea, Phialophora, Paecilomyces, Trichuris sp., Toxocara sp., Leishmania sp., B.equi, Thelazia sp., Paragonimus sp.

19. The kit of claim 16, wherein the nucleic acid probes are selected from between about 10 to about 30 microbes and comprise about 3 to about 5 probes per microbe.


SEQUENCE LISTING (160 sequences)

SEQ ID NO: 1 - DNA, 45 bp, Artificial Sequence
  Probe
tttctcgctctcacccttaacccgctggcgcgcctgcaccatctt

SEQ ID NO: 2 - DNA, 45 bp, Artificial Sequence
  Probe
cccgcactgacaccacacgtcatgcgcccccttgatttgcagtct

SEQ ID NO: 3 - DNA, 54 bp, Artificial Sequence
  Probe
gatgaatttacagacgcacaccggaatgcataagcaaccaaacgggatataaag

SEQ ID NO: 4 - DNA, 53 bp, Artificial Sequence
  Probe
accatgaacaaaactacaggaatcaagaacaaaacggaaggagcaggatctac

SEQ ID NO: 5 - DNA, 45 bp, Artificial Sequence
  Probe
caaaaacacggcaggaggggcctttttccacgagtaagactccat

SEQ ID NO: 6 - DNA, 52 bp, Artificial Sequence
  Probe
cttctaaactgtcgtttgatgcactagacgcacccccgactcaaattataga

SEQ ID NO: 7 - DNA, 45 bp, Artificial Sequence
  Probe
gtaaaaccaccactcgttggcaccctgcttcaccgcaactcccaa

SEQ ID NO: 8 - DNA, 45 bp, Artificial Sequence
  Probe
gcggccctcctcgccgcccaagaaggccacggggatctccttgta

SEQ ID NO: 9 - DNA, 45 bp, Artificial Sequence
  Probe
ctatatagcaggagagggagacccgacagccggtgtttttgaaca

SEQ ID NO: 10 - DNA, 45 bp, Artificial Sequence
  Probe
gcagcgcgtggccctgccagtcgccgcagtcgcaccacacgtcgt

SEQ ID NO: 11 - DNA, 45 bp, Artificial Sequence
  Probe
ctttgtctccaaggggaccccgcgccgcgccgtctgctacatcat

SEQ ID NO: 12 - DNA, 45 bp, Artificial Sequence
  Probe
cttaaacggacagcccctgggagaaacctcctactacggcggttg

SEQ ID NO: 13 - DNA, 45 bp, Artificial Sequence
  Probe
aaacccctcgagccgatcctcgtccgtgtcgctgttccagaacca

SEQ ID NO: 14 - DNA, 45 bp, Artificial Sequence
  Probe
accaggaaggaccaggcaaacaccaacgcccgcttcgagaacacg

SEQ ID NO: 15 - DNA, 45 bp, Artificial Sequence
  Probe
gcgaggagcagcaggatcaggtcggcgtgtccccacgcgtccgcg

SEQ ID NO: 16 - DNA, 45 bp, Artificial Sequence
  Probe
ctgcacgaagaggatcgccccggcgcccgtctcccacgccgcggg

SEQ ID NO: 17 - DNA, 45 bp, Artificial Sequence
  Probe
gagatcgtgccctcgacgcccgccatgctgggcctggggacccgc

SEQ ID NO: 18 - DNA, 45 bp, Artificial Sequence
  Probe
ctgcgtcacctgccggcgcgcgcgggcgtggcgggccgttaaaag

SEQ ID NO: 19 - DNA, 45 bp, Artificial Sequence
  Probe
gaagacgctgatgaaccacgagggcgaggtggggcagaggaagac

SEQ ID NO: 20 - DNA, 45 bp, Artificial Sequence
  Probe
ctggatctgctcctccaggcacttgatgacctgcttcttaaacag

SEQ ID NO: 21 - DNA, 45 bp, Artificial Sequence
  Probe
gctcctggcaaactatgtcaccaggctccccaaccagagaaacgc

SEQ ID NO: 22 - DNA, 46 bp, Artificial Sequence
  Probe
tatttgcaaagggaggcgaggagatggagtgactgaaggagcgata

SEQ ID NO: 23 - DNA, 45 bp, Artificial Sequence
  Probe
atctctgccgccatcccggccaggaaggcctcgatgaccgagtct

SEQ ID NO: 24 - DNA, 51 bp, Artificial Sequence
  Probe
caacctctgctcccctctattctcctcttgcgttatctccaatagaatttg

SEQ ID NO: 25 - DNA, 45 bp, Artificial Sequence
  Probe
gaacagaccgactccgggcgcgaggaggacgcacaggagagcgag

SEQ ID NO: 26 - DNA, 45 bp, Artificial Sequence
  Probe
cgtccaccgtccctctcacccccactcgaatcgcgcaggcgcgtc

SEQ ID NO: 27 - DNA, 45 bp, Artificial Sequence
  Probe
ggcaagcacctcgtttattgggaccggggctgtccggcgtctatt

SEQ ID NO: 28 - DNA, 45 bp, Artificial Sequence
  Probe
caatcagtgcgcccgatctcccggccactgaaccacaacggcatg

SEQ ID NO: 29 - DNA, 45 bp, Artificial Sequence
  Probe
agcacaacgcagactccgcctagactcccgcctccatccgctgac

SEQ ID NO: 30 - DNA, 45 bp, Artificial Sequence
  Probe
ataggccagagccacttccagaagcgcagcaagataaaggtgaac

SEQ ID NO: 31 - DNA, 45 bp, Artificial Sequence
  Probe
caaacacaacgtgaccccccgggagaccgtcctggatggcgatac

SEQ ID NO: 32 - DNA, 54 bp, Artificial Sequence
  Probe
ataataaaaacgataacacagaagaccccacacaccttgttgcatctaggctgc

SEQ ID NO: 33 - DNA, 57 bp, Artificial Sequence
  Probe
attttatccaaccggcaccaaacagggtagacttgttattcaaagatatacccgaat

SEQ ID NO: 34 - DNA, 45 bp, Artificial Sequence
  Probe
ctacacggtggacacccgggccggagagcgcacccgcgttccact

SEQ ID NO: 35 - DNA, 45 bp, Artificial Sequence
  Probe
cacaggcggcgtggcgatcctgccctcatccgtctcgcttaatcg

SEQ ID NO: 36 - DNA, 50 bp, Artificial Sequence
  Probe
aaacaagcagacatgatgatgagcatggggagacattagtgtggcagttt

SEQ ID NO: 37 - DNA, 50 bp, Artificial Sequence
  Probe
cagaaactactacaggcccgaggacacactaatagccctctaggagatat

SEQ ID NO: 38 - DNA, 45 bp, Artificial Sequence
  Probe
cataccactctaaaccctgcaatcctgcccagccagtttgttcat

SEQ ID NO: 39 - DNA, 45 bp, Artificial Sequence
  Probe
ccaacattccaccctccttcctccaggccatgcgcaaatactccc

SEQ ID NO: 40 - DNA, 45 bp, Artificial Sequence
  Probe
gtcatggcccggcgctgcgcccgcagcagcacgcaccgctccatg

SEQ ID NO: 41 - DNA, 45 bp, Artificial Sequence
  Probe
gacgtggtgcggtcgctcatcacctccacgctgcagcgggccggc

SEQ ID NO: 42 - DNA, 45 bp, Artificial Sequence
  Probe
gttcttccggaagacgacccgctccacggcgtccaccatgtccac

SEQ ID NO: 43 - DNA, 45 bp, Artificial Sequence
  Probe
ctgctccggcactccaccgagcgccgccacctattcgtcgacttc

SEQ ID NO: 44 - DNA, 60 bp, Artificial Sequence
  Probe
taatatcttctggaaggtttgtattctgaatggatccaccatctgccataatcctattct

SEQ ID NO: 45 - DNA, 60 bp, Artificial Sequence
  Probe
taaagacactccacatgccgtcactacctccgttagaagacatattaataagacttaaga

SEQ ID NO: 46 - DNA, 47 bp, Artificial Sequence
  Probe
taatagaggaaatcccaccgcctttctggatctcaccaacgacgata

SEQ ID NO: 47 - DNA, 45 bp, Artificial Sequence
  Probe
gatgatgcccttggcctcgcggtcgaagacggccacctcgctcac

SEQ ID NO: 48 - DNA, 45 bp, Artificial Sequence
  Probe
agacacttgaagtcgacgccggactcgccgcgcagcaccgagcgc

SEQ ID NO: 49 - DNA, 45 bp, Artificial Sequence
  Probe
tatggattcggctatccagtccttgaccgagcccacgatgcccgc

SEQ ID NO: 50 - DNA, 45 bp, Artificial Sequence
  Probe
gtccgcgtagcccgcgcccacggccttgccgcagtccgcgatcat

SEQ ID NO: 51 - DNA, 48 bp, Artificial Sequence
  Probe
gaagagttttcacaaaaagttttcgggaggagaggctgacctaccttc

SEQ ID NO: 52 - DNA, 45 bp, Artificial Sequence
  Probe
ggcgggagggaggggtctcgactgcgggcggtcctttttcacttt

SEQ ID NO: 53 - DNA, 45 bp, Artificial Sequence
  Probe
gatcaagaacaagacgcgcgtgcccttcctgctgctctcggcctc

SEQ ID NO: 54 - DNA, 45 bp, Artificial Sequence
  Probe
aacgaccctggctaccactcgcgggagactctctgcagcggacct

SEQ ID NO: 55 - DNA, 58 bp, Artificial Sequence
  Probe
tctttctcttcttcgctacatctgatgtcgatagacacctcacagtctttgatcatag

SEQ ID NO: 56 - DNA, 56 bp, Artificial Sequence
  Probe
ctatcaataactggcacaacaataacaggagttttcgccgccgccatttagttatt

SEQ ID NO: 57 - DNA, 60 bp, Artificial Sequence
  Probe
attacgaagaagacgacgaggacggagacggtagaataagtgtagcaaataaaatctata

SEQ ID NO: 58 - DNA, 45 bp, Artificial Sequence
  Probe
taacagccagtaaacaaagcacaaggggaagtggaaagcagccaa

SEQ ID NO: 59 - DNA, 45 bp, Artificial Sequence
  Probe
cgtccggtctccataacaacacatcctcccgctctgtgttctcac

SEQ ID NO: 60 - DNA, 48 bp, Artificial Sequence
  Probe
ttagactctacaaaaggcaggagatgagggacatgacaatggctcagt

SEQ ID NO: 61 - DNA, 45 bp, Artificial Sequence
  Probe
cttgacattgtgtgtcctgcctgtgccaagcaacgagaacgaaat

SEQ ID NO: 62 - DNA, 55 bp, Artificial Sequence
  Probe
gttaaagaagcaaactatgttaaaccaccagcaggaggcagacacctttgaatta

SEQ ID NO: 63 - DNA, 52 bp, Artificial Sequence
  Probe
atgagacagaggaagaaggggactggaaggttattgcaaacttccttagata

SEQ ID NO: 64 - DNA, 58 bp, Artificial Sequence
  Probe
atatgatggaaattgggtttggggctgcaaatttcaaggccttaaatcagtctaaatc

SEQ ID NO: 65 - DNA, 50 bp, Artificial Sequence
  Probe
atagatgaggaaggggactggaagcacatagggaactttcttagattcca

SEQ ID NO: 66 - DNA, 45 bp, Artificial Sequence
  Probe
gactgtggaggagggtgcaggatagagtctggaaagattgtctct

SEQ ID NO: 67 - DNA, 45 bp, Artificial Sequence
  Probe
gcactccttgagcctctcccccttgaccctcatcttcttgacaag

SEQ ID NO: 68 - DNA, 45 bp, Artificial Sequence
  Probe
agatctctccgggtggctcctgttgaccggggtggccgtccagtt

SEQ ID NO: 69 - DNA, 45 bp, Artificial Sequence
  Probe
atcaagatgagcaagattggaaagggctgcaccctcgtcatggcg

SEQ ID NO: 70 - DNA, 50 bp, Artificial Sequence
  Probe
tttccatagacgacgtggacgcgtttgtgtctgttttgacggtttttaaa

SEQ ID NO: 71 - DNA, 45 bp, Artificial Sequence
  Probe
acatccatggctcgccgtctgcttctctgccgctcgtggtgccga

SEQ ID NO: 72 - DNA, 45 bp, Artificial Sequence
  Probe
ggacgctgctacaaccaccgtgtcgtccgcgttcgtcgtccccag

SEQ ID NO: 73 - DNA, 45 bp, Artificial Sequence
  Probe
gtctcgcggcggctccctctcggcggctccggttgggctcccctc

SEQ ID NO: 74 - DNA, 45 bp, Artificial Sequence
  Probe
gaccacatcccgctcctgctcatcgtcacgcccgtggtctttgac

SEQ ID NO: 75 - DNA, 45 bp, Artificial Sequence
  Probe
aaaggggttggacatgaaggaggacacgcccgacacggccgatac

SEQ ID NO: 76 - DNA, 45 bp, Artificial Sequence
  Probe
atcccctcgaagaacgcgcccaggcccgcaaacatggcggcgttg

SEQ ID NO: 77 - DNA, 45 bp, Artificial Sequence
  Probe
gaccccaggcgtgccgggggaactcggagccgccgacgccaccag

SEQ ID NO: 78 - DNA, 45 bp, Artificial Sequence
  Probe
cggagtggcagggcccccgttcgccgcctgggtcgcggccgcgac

SEQ ID NO: 79 - DNA, 45 bp, Artificial Sequence
  Probe
atatacctcccgaacaccatgaggaacccacctcatcctctggat

SEQ ID NO: 80 - DNA, 46 bp, Artificial Sequence
  Probe
tctggatccagtagcagagaggagaccaccaattcaggaagagaat

SEQ ID NO: 81 - DNA, 49 bp, Artificial Sequence
  Probe
gtttacagattaggaatacatatcctcctccttcaccaccccgaagacc

SEQ ID NO: 82 - DNA, 45 bp, Artificial Sequence
  Probe
gaatatgggcccaatccacacggggccaactcaagatccagaaag

SEQ ID NO: 83 - DNA, 45 bp, Artificial Sequence
  Probe
tatgatcatgaacagactgtgaggactgaggggcctgaaatgagc

SEQ ID NO: 84 - DNA, 45 bp, Artificial Sequence
  Probe
taattaacaggaggacacagagggtggatgggcagcctatgattg

SEQ ID NO: 85 - DNA, 54 bp, Artificial Sequence
  Probe
agcagtagcctcatcatcactagatggcatttcttctgagcaaaacaggttttc

SEQ ID NO: 86 - DNA, 51 bp, Artificial Sequence
  Probe
ttcagggggaggtgtgggaggttttttaaagcaagtaaaacctctacaaat

SEQ ID NO: 87 - DNA, 51 bp, Artificial Sequence
  Probe
ttttcctcattaaaggcattccaccactgctcccattcatcagttccatag

SEQ ID NO: 88 - DNA, 45 bp, Artificial Sequence
  Probe
aacgcgtcacctcatccgcccgatggctatccaaaaccgccacct

SEQ ID NO: 89 - DNA, 45 bp, Artificial Sequence
  Probe
cttcggtccaaacaactcacctgctccgaaatccgaatcttccaa

SEQ ID NO: 90 - DNA, 45 bp, Artificial Sequence
  Probe
ttcaacacctcctccgaactcgccccttttcctccttccgcgtct

SEQ ID NO: 91 - DNA, 45 bp, Artificial Sequence
  Probe
gagaaaccagcaacggagcggcgaatcgacaagggagaaacaact

SEQ ID NO: 92 - DNA, 45 bp, Artificial Sequence
  Probe
ctcatcgaccacctgctgcagagccagcggcccatcacccgcaag

SEQ ID NO: 93 - DNA, 45 bp, Artificial Sequence
  Probe
cgtgagttaggtcgagcagagccaaagcccccggtgcttcgtcgc

SEQ ID NO: 94 - DNA, 45 bp, Artificial Sequence
  Probe
ttgccttgcgccttccctgaccagggggtgagtttttctccaaaa

SEQ ID NO: 95 - DNA, 45 bp, Artificial Sequence
  Probe
gagagtgtcctacacttaggggagaagcagccaaggggttgtttc

SEQ ID NO: 96 - DNA, 45 bp, Artificial Sequence
  Probe
accttcctcctgaggcaaggaccacagccaacttcctcttacaag

SEQ ID NO: 97 - DNA, 45 bp, Artificial Sequence
  Probe
caggagcgatggcagaggccagggaaaaaggagatttgactttta

SEQ ID NO: 98 - DNA, 59 bp, Artificial Sequence
  Probe
gaaagatttttcattataccaaggagggggcagtggctagacaattagaacacatttct

SEQ ID NO: 99 - DNA, 48 bp, Artificial Sequence
  Probe
aacagtaaaccctgttccgactactgcctcacccatatcgtcaatctt

SEQ ID NO: 100 - DNA, 46 bp, Artificial Sequence
  Probe
gcgctttccaccggatactctggcaactttgactcagttactgatt

SEQ ID NO: 101 - DNA, 45 bp, Artificial Sequence
  Probe
tctcttgcctgactgtgcccgcttcagcctaccaagtgcgcaatt

SEQ ID NO: 102 - DNA, 45 bp, Artificial Sequence
  Probe
gcaggagatgggcggcaacatcaccagggttgagtcagagaacaa

SEQ ID NO: 103 - DNA, 45 bp, Artificial Sequence
  Probe
acccataccagggtctcgcccagtggcacgcctaggattatatag

SEQ ID NO: 104 - DNA, 45 bp, Artificial Sequence
  Probe
gaagaaacacagacgactatccagcgaccaagatcagagccagac

SEQ ID NO: 105 - DNA, 47 bp, Artificial Sequence
  Probe
acacatctgcttgtgctactgctcttcctgtggctctctcaactaac

SEQ ID NO: 106 - DNA, 45 bp, Artificial Sequence
  Probe
tagacctaaacagtccagaggagcaggacgacaatggaaacactg

SEQ ID NO: 107 - DNA, 45 bp, Artificial Sequence
  Probe
caccataggccctcgcaaacgttctgctccatctgccactacgtc

SEQ ID NO: 108 - DNA, 45 bp, Artificial Sequence
  Probe
tttccaaagcctctgctgcccctaaacgtaagcgcgccaaaacta

SEQ ID NO: 109 - DNA, 45 bp, Artificial Sequence
  Probe
gtccaaggcaccctgggtcctcttacgaatgtctgactacttcag

SEQ ID NO: 110 - DNA, 45 bp, Artificial Sequence
  Probe
gtaagagggagacccaaaggcggcggcactaaagattgttctggt

SEQ ID NO: 111 - DNA, 45 bp, Artificial Sequence
  Probe
ttcttgaaaaggacgaccagcacatggagcagcaggttatggcaa

SEQ ID NO: 112 - DNA, 45 bp, Artificial Sequence
  Probe
agtcatccctgttacagtctccgggaagggcctttgcacccgtta

SEQ ID NO: 113 - DNA, 45 bp, Artificial Sequence
  Probe
gaatcccttaaagccagtctcagttcggattggggtctgcaactc

SEQ ID NO: 114 - DNA, 45 bp, Artificial Sequence
  Probe
cgtggcctaactcgtttgagggggagcggacgaaggtgggattag

SEQ ID NO: 115 - DNA, 45 bp, Artificial Sequence; identical to SEQ ID NO: 113
  Probe

SEQ ID NO: 116 - DNA, 54 bp, Artificial Sequence
  Probe
gagttgcagaggacaatccgaactgagacaattttaaggattaaccctctgtag

SEQ ID NO: 117 - DNA, 45 bp, Artificial Sequence
  Probe
aaagccacgtctccgtgcggtccaggcatgtcaaaaggtggtaag

SEQ ID NO: 118 - DNA, 45 bp, Artificial Sequence
  Probe
caactcgaccccatgaagttggagtcgctagtaatcgcagatcag

SEQ ID NO: 119 - DNA, 45 bp, Artificial Sequence
  Probe
gaatctcaaaaagccagtctcagttcggattggggtctgcaactc

SEQ ID NO: 120 - DNA, 55 bp, Artificial Sequence
  Probe
aatatgatgctaatctctaaaagccattcacagttcggattggggtctgcaactc

SEQ ID NO: 121 - DNA, 45 bp, Artificial Sequence
  Probe
gggaacttcggtccttgcgctatcggatgaacccatatgggatta

SEQ ID NO: 122 - DNA, 45 bp, Artificial Sequence
  Probe
cctgagagggtgaacggccacattggaactgagaaacggtccaaa

SEQ ID NO: 123 - DNA, 54 bp, Artificial Sequence
  Probe
gatagcaagcgaatctcaaaaagcctatctcagttcggattgttctctgcaact

SEQ ID NO: 124 - DNA, 45 bp, Artificial Sequence
  Probe
caacggcccaccaaggcgacgatcagtaggggttctgagaggaag

SEQ ID NO: 125 - DNA, 45 bp, Artificial Sequence
  Probe
gaaccttacccgggcttgaattgcaggtgctgcccacagagacgt

SEQ ID NO: 126 - DNA, 45 bp, Artificial Sequence
  Probe
gaatcccaaaaagccgctctcagttcggattgcaggctgcaactc

SEQ ID NO: 127 - DNA, 45 bp, Artificial Sequence
  Probe
gatcccagaccccggctttgcgccagcacacgaagcggttgtaac

SEQ ID NO: 128 - DNA, 45 bp, Artificial Sequence; identical to SEQ ID NO: 118
  Probe

SEQ ID NO: 129 - DNA, 60 bp, Artificial Sequence
  Probe
gggcgtctaagttaccaattctcgtctgatggctacatacggcggtcagtttacgcttac

SEQ ID NO: 130 - DNA, 45 bp, Artificial Sequence
  Probe
atgaaagccggcgacacccgaagcccgtggccctgtggggagcgg

SEQ ID NO: 131 - DNA, 45 bp, Artificial Sequence
  Probe
ctaatccctaaaagccggtctcagttcggattggggtctgcaact

SEQ ID NO: 132 - DNA, 52 bp, Artificial Sequence
  Probe
gttaagtcctataacgagcgcaacccctgcgaatagttgccatcattaagtt

SEQ ID NO: 133 - DNA, 45 bp, Artificial Sequence
  Probe
cttcttgaccaggctcacttcgccgccgacgggccagcatcgctt

SEQ ID NO: 134 - DNA, 45 bp, Artificial Sequence
  Probe
aaacgaagcccgggcgagtaggcaggcgcgggggccgtgacgaag

SEQ ID NO: 135 - DNA, 45 bp, Artificial Sequence
  Probe
ccgcagagggtgatagccccgtaaccggcgacagcgagggagtag

SEQ ID NO: 136 - DNA, 45 bp, Artificial Sequence
  Probe
cgaacgaactgcgaatgagcctggcgcggcgtgcgttttaatgac

SEQ ID NO: 137 - DNA, 45 bp, Artificial Sequence
  Probe
gatgcgcctctagaggtaggggggcggaccgatgctgcagaaggc

SEQ ID NO: 138 - DNA, 45 bp, Artificial Sequence
  Probe
gatagagaaacaggggtgtgttcctgtcccgcgctgccgtgcggc

SEQ ID NO: 139 - DNA, 46 bp, Artificial Sequence
  Probe
aggtctcctaggtgaatagcctctggttgatgttgaacgcaggtaa

SEQ ID NO: 140 - DNA, 45 bp, Artificial Sequence
  Probe
cttaatctgaccgccggaggaccgcctaatacgggtgttgcctct

SEQ ID NO: 141 - DNA, 45 bp, Artificial Sequence
  Probe
ttgctttggcggacccgtctcacgaccgccctgggaccgctgaaa

SEQ ID NO: 142 - DNA, 45 bp, Artificial Sequence
  Probe
aaatgacttggcggcctcgtcgcggccctcctctgcgtagtatag

SEQ ID NO: 143 - DNA, 45 bp, Artificial Sequence
  Probe
aacttgcttgccgcgtcctcctcgcgccctgcaaccaggcctctc

SEQ ID NO: 144 - DNA, 45 bp, Artificial Sequence
  Probe
ctgctctaagatcttcgctgctgaggcccgcgccgccgctcttcc

SEQ ID NO: 145 - DNA, 45 bp, Artificial Sequence
  Probe
aaagaagaagataggggcagagggggagtgagcctcgtcgtcgac

SEQ ID NO: 146 - DNA, 45 bp, Artificial Sequence
  Probe
caacggaatccagtgcccaccggagcgccagttcgtgcgagagtt

SEQ ID NO: 147 - DNA, 45 bp, Artificial Sequence
  Probe
cttccgtctctaccctcccgaggcgcttttctcactgaccgactt

SEQ ID NO: 148 - DNA, 45 bp, Artificial Sequence
  Probe
actctcacgcccacccgcacggctgctccgagggaggggctctct

SEQ ID NO: 149 - DNA, 57 bp, Artificial Sequence
  Probe
acgacgacaacgcacagaaatattagtagtaaaccggctgctcattggaaatacttt

SEQ ID NO: 150 - DNA, 45 bp, Artificial Sequence
  Probe
aattcgggcgtgtttttcaccaaatcccacatggccgggctacta

SEQ ID NO: 151 - DNA, 52 bp, Artificial Sequence
  Probe
cgacaacgacaactctatgataatagacttgtgttccgacgcgcgcataatc

SEQ ID NO: 152 - DNA, 45 bp, Artificial Sequence
  Probe
gtttgtttatgatcttggaggcggacaaggcggtgttgttgtgtg

SEQ ID NO: 153 - DNA, 45 bp, Artificial Sequence
  Probe
tatttcatcacaacgttgttgcacatgagcaggctggacacgacc

SEQ ID NO: 154 - DNA, 47 bp, Artificial Sequence
  Probe
aaacttttttactgccgtctttgttacacgcacgccgactggttgtg

SEQ ID NO: 155 - DNA, 45 bp, Artificial Sequence
  Probe
gcgtggtgaccgagaccgctgtagatggccctgatgcagtgatcc

SEQ ID NO: 156 - DNA, 45 bp, Artificial Sequence
  Probe
ctcgtggctgtggggtgccagatctgtggcgtttccctaacatat

SEQ ID NO: 157 - DNA, 49 bp, Artificial Sequence
  Probe
taaccataaacgatgccgactagagattggaggtcgtcagtttgaacga

SEQ ID NO: 158 - DNA, 49 bp, Artificial Sequence
  Probe
taacccgttgaaaatcctccgtgatcgggatcgggaattgcaattattt

SEQ ID NO: 159 - DNA, 51 bp, Artificial Sequence
  Probe
ctaattccgatatcgaacgagactctggcctactaactagcggcggtatta

SEQ ID NO: 160 - DNA, 60 bp, Artificial Sequence
  Probe
ctcgccggcccgccgccgatgatgatgatgaagcgacagcctccaacaacaataatgata